(19) United States
(12) Patent Application Publication
(10) Pub. No.: US 2025/0282849 A1
(43) Pub. Date: Sep. 11, 2025

(54) FNDC4 FUSION PROTEIN AND USES THEREOF

(71) Applicant: Helmholtz Zentrum München - Deutsches Forschungszentrum für Gesundheit und Umwelt (GmbH), Neuherberg (DE)

(72) Inventors: Anastasia GEORGIADI DAMMAN, Munich (DE); Stephan HERZIG, Vaterstetten (DE)

(21) Appl. No.: 18/273,961

(22) PCT Filed: Jan. 25, 2022
(86) PCT No.: PCT/EP2022/051561

§ 371 Date: Jul. 24, 2023

(30) Foreign Application Priority Data
Jan. 25, 2021 (LU) 102453

(51) Int. Cl. C07K 14/78; A61K 38/39; A61P 3/10

(52) U.S. Cl. CPC C07K 14/78; A61K 38/39; A61P 3/10; C07K 2319/30; C07K 2319/50

(57) ABSTRACT

The present invention relates to a fusion protein (FcsFNDC4) comprising a) a soluble FNDC4 (sFNDC4) or a functional fragment thereof; b) a peptide linker; and c) a Fc-domain. The present invention further relates to a nucleic acid molecule comprising a nucleotide sequence encoding said fusion protein, a vector comprising said nucleic acid molecule, and a host cell comprising the vector or the nucleic add molecule. The present invention also relates to a fusion protein for use in therapy. In particular, the present invention relates to a fusion protein for use in a method of preventing and/or treating diabetes or inflammation in a subject. The invention also relates to a composition comprising at least one fusion protein. Further, the present invention relates to a kit comprising said fusion protein or said composition. The invention also comprises a method of producing the fusion protein and a method of stratifying a subject with diabetes applying the fusion protein of the invention.

### PRIORITY CLAIM

This application claims priority to International Application No. PCT/EP2022/051561, filed Jan. 25, 2022 which claims priority to Luxembourg Patent Application No. 102453, filed Jan. 25, 2021, wherein the contents of said applications are incorporated herein by reference in their entireties

### REFERENCE TO SEQUENCE LISTING

This application contains a Sequence Listing in a computer readable form, submitted via EFS-Web. The entire contents of the ASCII text file entitled “IPM0159US_Sequence_Listing_V2.txt” created on Jun. 28, 2024, and having a size of 17,519 bytes, is incorporated herein by reference.

### TECHNICAL FIELD OF THE INVENTION

The present invention relates to a fusion protein (FcsFNDC4) comprising a) a soluble FNDC4 (sFNDC4) or a functional fragment thereof; b) a peptide linker; and c) a Fc-domain. The present invention further relates to a nucleic acid molecule comprising a nucleotide sequence encoding said fusion protein, a vector comprising said nucleic acid molecule, and a host cell comprising the vector or the nucleic acid molecule. The present invention also relates to a fusion protein for use in therapy. In particular, the present invention relates to a fusion protein for use in a method of preventing and/or treating diabetes or inflammation in a subject. The invention also relates to a composition comprising at least one fusion protein. Further, the present invention relates to a kit comprising said fusion protein or said composition. The invention also comprises a method of producing the fusion protein and a method of stratifying a subject with diabetes applying the fusion protein of the invention.

### BACKGROUND OF THE INVENTION

Type 2 diabetes (T2D) is a gradually developing disease in which genetic, lifestyle, and ageing factors each may separately, or in combination, accelerate its progression and severity. Early glucose intolerance is a hallmark of the pre diabetic state and it is targeted therapeutically by the prescription of metformin or lifestyle changes, such as diet and exercise (Barry E., et al. (2017), Review BMJ, 356:i6538, Fonseca, V. A. (2009), Diabetes Care 32, 151-156). However, there is a lack of mechanistic understanding of pre-diabetes development, as well as the endocrine and molecular factors that can explain the individual responsiveness to therapeutic interventions.

Of the plethora of biological actions elicited by insulin, the inability to remove excess glucose from the blood circulation is a central step in the pathogenesis of obesity related T2D. Progressive insulin resistance and the subsequent failure to cope with dietary glucose, i.e. glucose intolerance, mostly reflects the inability of adipose tissue and skeletal muscle to sufficiently eliminate circulating glucose in response to the hormone. In this pre-diabetic state, improvements in insulin sensitivity are in principal still able to delay or even prevent the onset of full blown diabetes, making this disease stage an attractive target for tailored interventions and preventive measures. However, mechanisms contributing to the glucose-intolerant, pre-diabetic phenotype remain vastly unclear.

Recent research progress has clearly promoted the concept that systemic glucose homeostasis is determined by a variety of inter-tissue communication pathways, and most peripheral organs have been described to exhibit a secretory, endocrine function, including the liver. Indeed, hepatic steatosis is the strongest predictor of insulin resistance in skeletal muscle and adipose tissue, tightly coupled to alterations in the hepatic secretory function and the release of so-called hepatokines that are capable of controlling distant metabolic processes (Meex, R. C. R., and Watt, M. J. (2017). Hepatokines: linking nonalcoholic fatty liver 782 disease and insulin resistance. Nat. Rev. Endocrinol. 13, 509-520). Understanding of specific endocrine routes by which the liver regulates insulin action in distinct peripheral organs has just emerged, with critical mechanisms remaining to be discovered.

A protein involved in the insulin resistance, although by yet undiscovered mechanisms, is Fibronectin type Ill domain-containing protein 4 (FNDC4) is a type I transmembrane protein, releasing a soluble bioactive protein (sFNDC4) that is highly conserved amongst mouse and primates (Bosma, M., et al. (2016), Nat. Commun. 7). Previous work in animal models has shown that sFNDC4 exerts anti-inflammatory effects on macrophages and osteoclasts promoting survival in response to severe chronic inflammation. Exploration of the FNDC4 biology is in its early stages and many questions remain to be addressed in order to determine whether this secreted protein acts as a hormone and has therapeutic potential, especially for metabolic diseases such as diabetes. So far, it is still unclear whether and how sFNDC4 acts in an endocrine manner to maintain glucose homeostasis. Thus, basic target biology and receptor pharmacology is unknown in this regard and therefore still requires better understanding of this biological system.

It is therefore an object of the present invention to provide new insights in the control of glucose homeostasis with regard to sFNDC4 and to provide alternative therapeutic strategies for diabetes.

Therefore, the objective of the present invention is to comply with this need.

The solution of the present invention is described in the following, exemplified in the appended examples, illustrated in the figures and reflected in the claims.

### SUMMARY OF THE INVENTION

The present invention deals with a soluble FNDC4 (sFNDC4) fusion protein fused to a Fc domain which has been purified from mammalian CHO-S cells, and subsequently used for biological and pharmacological proof of concept studies with regard to diabetes. The inventors introduced a peptide linker comprising a TEV protease site between the Fc-domain and sFNDC4, which refers to Fc-sFNDC4. This particular modified protein fused to a Fc-domain (see FIG. 1) was then tested in different in vitro and in vivo experiments with regard to maintaining glucose homeostasis. It has also been shown that endogenous sFNDC4 is positively associated with glucose tolerance as well as insulin sensitivity in humans (see FIG. 2). Additionally, in vivo data showed that mice with hepatic deletion of FNDC4 exhibited decreased circulating levels of sFNDC4 and developed a prediabetes phenotype (see FIG. 3).

Further, in vivo data demonstrated that after having applied the fusion protein of the present invention and a Fc-fused sFNDC4 from the prior art (i. e without a linker according to the present invention) in particular doses, the fusion protein of the present invention tends to improve glucose tolerance in comparison to the endogenous sFNDC4 protein of the prior art not being modified according to the present invention (see FIG. 4). Also it was additionally found out by the present inventors that when applying the fusion protein of the present invention as defined elsewhere herein at a dose below 3 mg/kg, said new dosage regimen of the fusion protein, which is about 15 times lower than as taught by the prior art, is of additional advantage. In particular, when high fat diet (HFD) mice were injected (i.p.) with about 0.2 mg/kg in a certain treatment regimen, an improved glucose tolerance without any effect on body weight or food intake was observed. However, when HFD mice were treated with higher doses of Fc-sFNDC4 (up to 3 mg/kg as it is used in the prior art), no effect on glucose tolerance was observed (see FIG. 5), thereby supporting the fact that the novel fusion protein of the present invention can be applied in in vivo approaches with regard to treating diabetes. The inventors also found that the total amount of injected Fc-sFNDC4 entered the circulation within 48 hrs post injection when administered subcutaneously (FIG. 10), so that subcutaneous administration is considered an appropriate administration route. In addition, Fc-sFNDC4 also showed 100% bioavailability, meaning no loss of Fc-sFNDC4 protein. Further, after subcutaneous administration Fc-sFNDC4 is stable for a long time in the blood circulation having a half-life being determined to be at least about 192 hours or at least about 8 days. Additionally, it could be further demonstrated that the GPR116 is a functional receptor of said fusion protein in vitro and in vivo supporting the notion that said fusion protein primarily targets the adipocytes via GPR116. First of all, the inventors identified with in vitro experiments that GPR116 is a candidate receptor for sFNDC4 (see FIG. 6). In vivo experiments further showed that in mice lacking the GPR116, the fusion protein Fc-sFNDC4 of the invention failed to improve glucose tolerance (see FIG. 7) and thus that Fc-sFNDC4 effects are mediated exclusively by adipose tissue GPR116 (see FIG. 7).

In sum, the present invention provides for a novel fusion protein which can be used as a therapeutic substantially improving glucose tolerance in vitro and in vivo, thus having tremendous potential in diagnosis and therapeutics.

In a first aspect, the present invention relates to a fusion protein comprising a) a soluble FNDC4 (sFNDC4) or a functional fragment thereof; b) a peptide linker; and c) a Fc-domain.

The present invention may also comprise the fusion protein as defined elsewhere herein, wherein the sFNDC4 comprises an amino acid sequence having at least 70% identity with an amino acid sequence of SEQ ID NO.: 1.

The present invention may also comprise the fusion protein as defined elsewhere herein, wherein the sFNDC4 comprises an amino acid sequence of SEQ ID NO.: 1.

The present invention may also envisage the fusion protein as defined elsewhere herein, wherein the sFNDC4 has the amino acid sequence of SEQ ID NO.: 2.

The present invention may also envisage the fusion protein as defined elsewhere herein, wherein the functional fragment is at least about 10 amino acids long.

The present invention may also relate to the fusion protein as defined elsewhere herein, wherein the C-terminal residue of the peptide linker is directly fused to the N-terminus of the sFNDC4.

The present invention may also envisage the fusion protein as defined elsewhere herein, wherein the N-terminal residue of the peptide linker is directly fused to the C-terminal residue of the Fc-domain.

The present invention may also comprise the fusion protein as defined elsewhere herein, wherein the peptide linker comprises between about 5 and about 13 amino acids, preferably about 9 amino acids.

The present invention may also relate to the fusion protein as defined elsewhere herein, wherein the peptide linker comprises a Tobacco Etch Virus (TEV) protease site.

The present invention may also envisage the fusion protein as defined elsewhere herein, wherein the peptide linker comprises the amino acid sequence of SEQ ID NO.: 3.

The present invention may also comprise the fusion protein as defined elsewhere herein, wherein the Fc-domain is selected from the group consisting of an IgG1, IgG2, IgG3 and an IgG4 Fc-domain.

The present invention may also envisage the fusion protein as defined elsewhere herein, wherein the Fc-domain is an IgG1 Fc-domain.

The present invention may also relate to the fusion protein as defined elsewhere herein, wherein the Fc-domain is a human Fc-domain or a mouse Fc-domain.

The present invention may also comprise the fusion protein as defined elsewhere herein, having binding affinity to the G-protein coupled receptor GPR116.

The present invention may also relate to the fusion protein as defined elsewhere herein, wherein the fusion protein specifically binds to the N-terminus of the GPR116 receptor.

The present invention may also envisage the fusion protein as defined elsewhere herein having the amino acid sequence of SEQ ID NO.: 4.

The present invention may also relate to the fusion protein as defined elsewhere herein having the amino acid sequence of SEQ ID NO.: 5.

In a second aspect, the present invention relates to a nucleic acid molecule comprising a nucleotide sequence encoding the fusion protein as defined elsewhere herein. The present invention also relates to a vector comprising said nucleic acid molecule and to a host cell comprising said nucleic acid molecule or said vector as defined elsewhere herein.

In a third aspect, the invention relates to said fusion protein as defined elsewhere herein for use in therapy.

In a fourth aspect, the present invention relates to said fusion protein as defined elsewhere herein for use in a method of preventing and/or treating diabetes in a subject, the method comprising administering to the subject a therapeutically effective amount of the said fusion protein.

The present invention may also envisage the fusion protein for the use as defined elsewhere herein, wherein the fusion protein is administered to the subject in a dosage below 3 mg/kg.

The present invention may also relate to the fusion protein for the use as defined elsewhere herein, wherein said administering is performed by injection or by infusion.

The present invention may also comprise the fusion protein for the use as defined elsewhere herein, wherein the administration is performed intraperitoneally, intravenously, intraarterially, subcutaneously or intramuscularly.

The present invention may also envisage the fusion protein for the use as defined elsewhere herein, wherein the administration is performed intraperitoneally.

The present invention may also envisage the fusion protein for the use as defined elsewhere herein, wherein said administering performed intraperitoneally comprises at least about 8 administrations, preferably at least about 8 administrations within one month.

The present invention may also envisage the fusion protein for the use as defined elsewhere herein, wherein the administration is performed subcutaneously.

The present invention may also envisage the fusion protein for the use as defined elsewhere herein, wherein said administering performed subcutaneously comprises administration once a week, preferably once a week within one month.

The present invention may also comprise the fusion protein for the use as defined elsewhere herein, wherein the fusion protein is administered in combination with an additional therapeutic agent.

The present invention may also envisage the fusion protein for the use as defined elsewhere herein, wherein the fusion protein improves glucose tolerance in the subject.

The present invention may also comprise the fusion protein for the use as defined elsewhere herein, wherein the fusion protein has binding affinity to the G-protein coupled receptor GPR116.

The present invention may also relate to the fusion protein for the use as defined elsewhere herein, wherein the fusion protein specifically binds to the N-terminus of the GPR116 receptor.

The present invention may also envisage the fusion protein for the use as defined elsewhere herein, wherein the fusion protein improves glucose tolerance by specifically binding to the GPR116 receptor.

The present invention may also relate to the fusion protein for the use as defined elsewhere herein, wherein the GPR116 receptor is located in adipose tissue cells.

The present invention may also envisage the fusion protein for the use as defined elsewhere herein, wherein the subject is a mammal, preferably a human.

In a fifth aspect, the present invention relates to a composition comprising at least one fusion protein as defined elsewhere herein. The present invention may also comprise said composition, which further comprises at least one diagnostically or pharmaceutically acceptable carrier.

In a sixth aspect, the present invention relates to a kit comprising said fusion protein or said composition as defined elsewhere herein.

In a seventh aspect, the invention relates to a method of producing the fusion protein of the invention, wherein the fusion protein is produced starting from the nucleic acid coding for the fusion protein by means of genetic engineering methods, wherein optionally the fusion protein is produced in a bacterial or eukaryotic host organism and is isolated from the host organism or its culture.

In an eighth aspect, the invention relates to a method of stratifying a subject with diabetes, comprising a) determining the level of sFNDC4 or a functional fragment thereof in a test sample obtained from said subject, which has been contacted with said fusion protein as defined elsewhere herein, and b) stratifying said subject as suffering from diabetes, if the level of sFNDC4 is decreased relative to a corresponding level of sFNDC4 in a control sample obtained from a healthy subject.

Finally, the invention relates to a fusion protein as defined elsewhere herein for use in a method of preventing and/or treating inflammation in a subject, the method comprising administering to the subject a therapeutically effective amount of the fusion protein as defined elsewhere herein.

### BRIEF DESCRIPTION OF THE FIGURES

FIG. 1: Protein sequence of SP-6His-TEV-sFNDC4 used in the present invention. 6×His-tag depicted in italic; Fc (IgG 1) depicted in underlined; TEV protease site (linker between Fc and hsFNDC4) depicted in bold; AA=additional amino acids for the linker depicted in dark grey; sFNDC4 depicted in light grey. As a signal peptide in an expression vector SEQ ID NO: 9 may be used as depicted.

FIG. 2: Liver and serum FNDC4 levels positively associate with glucose tolerance in humans. a) RT-qPCR quantification of mRNA levels of the mouse and the human Fndc4 gene in indicated tissues. The human data were retrieved from the Protein Atlas Project database, URL: http://www.proteinatlas.org/search/Fndc4. Pearson's correlation for human liver Fndc4 mRNA levels and fasting blood glucose levels (mmol/It) b) and c) blood glucose levels (mmol/It) 2 h post oral glucose tolerance test (OGTT). d) RT-qPCR quantification of human liver Fndc4 mRNA levels at indicated groups. ND: non-diabetic, T2D: Type 2 Diabetes, Obese-IGT/IIT: impaired glucose tolerance/impaired insulin tolerance (Methods: Cross-sectional study Leipzig). e) Serum levels of sFNDC4 ng/ml in paired human blood samples, that initially consumed a low fat (LF) diet for 6 weeks and subsequently were given a high fat (HF) diet for 6 weeks (Methods: NUGAT study-DIfE). Serum was collected at the end of the LF diet period (LF) and at 1 week (HF 1 wk) and 6 weeks (HF 6 wks) of HFD diet (paired samples).

Number of measured samples is indicated as (n). Data shown are mean+SEM. (*) indicates p<0.05, (**) indicates p<0.01, (***) indicates p<0.001 using Student's t-test. Paired Student's t-test was applied in e).

FIG. 3: Mice with hepatic deletion of FNDC4 exhibited decreased circulating levels of sFNDC4 and developed a prediabetes phenotype. a) Schematic representation of study protocol. b) RT-qPCR quantification of Fndc4 mRNA in indicated tissues (n=5 mice per group for the liver, n=5 per group for skeletal muscle (gastrocnemius) and n=7 mice per group for white adipose tissue (gWAT). c) Western blot against FNDC4 protein in the liver and VCP as loading control (n=2 mice per group are shown). d) Elisa quantification of plasma (trunk blood) sFNDC4 (n=5 mice per group), 3 weeks post iv. injection of AAVs. e) RT-qPCR quantification of Fndc4 mRNA at indicated tissues (n=5 mice per group) and f) ELISA quantified plasma (trunk blood) sFNDC4 (n=9 ChowAAVshControl, n=6 ChowAAVshFNDC4, n=8 HFDAAVshControl, n=8 HFDAAVshFNDC4) 11 weeks post i.v. injection of AAVs and 8 weeks on HFD (45% fat) or age matchedcontrol mice on chow diet. Glucose homeostasis in chow and HFD fed mice: g) blood glucose, h) plasma insulin levels during an IPGTT i) Blood glucose levels during an ITT on chow fed mice. j) Blood glucose levels, k) area under the curve (refers to j)) and I) plasma insulin during an IPGTT on HFD fed mice (weeks on HFD as shown on graph). m) Blood glucose levels during an ITT on HFD fed mice. Mice were C57BL6N, put on HFD when 10-11 weeks old and compared with age matched chow fed mice. Number of mice per group n=5-7. HFD contained 45% fat. During IPGTT and glucose induced insulin test 2 g/kg D-glucose was injected (i.p.). and during ITT 0.8 U/kg insulin was used (i.p.). Data shown are mean+SEM. (*) indicates p<0.05, (**) indicates p<0.01, (***) indicates p<0.001 using Student's t-test, ns; non-significant.

FIG. 4: Comparison of prior art Fc fused sFNDC4 (here called “FcsFNDC4-linker” minus linker) and FcsFNDC4 of the invention (here called “FcsFNDC4+linker” (plus linker) at 3 mg/kg i.p. injection every second day for 1 month.

a) Blood glucose levels during an IPGTT 2 g/kg D-glucose was injected after 6 hours fasting. b) Areas under the curve corresponding to the a). c) Fasting insulin levels. n=5-6 mice per group treated with depicted recombinant proteins for 1 month. *p<0.05, Student's test.

FIG. 5: Every second day injections of recombinant FcsFNDC4 0.2 mg/kg improved glucose tolerance and increased glucose uptake specifically in the white adipose tissue.

a) Plasma sFNDC4 in mice under HFD (16 weeks on HFD 60% fat) (n=4 mice per time point per group). Blood was collected from the central vein (trunk) after decapitation. b) RT-qPCR quantification of liver Fndc4 mRNA at 16 weeks HFD and chow control mice (n=4 per group). c) Plasma sFNDC4 ng/ml under chow, HFD (60% fat for 14 weeks) and HFD 48 h after a single i.p. injection of FcsFNDC4 (0.2 mg/kg). Blood was collected from the trunk of the mice. d) Blood glucose during IPGTT test at indicated time points (week=duration of FcsFNDC4 injections) and area under the curve e, f) Glucose stimulated insulin response during the IPGTT in (d and e) after 4 wks of FcsFNDC4 injections. g) Blood glucose as % of baseline glucose levels during an ITT. h) Quantification of 2-NBDG glucose content in different tissues, 35 min after i.p. injection of a mixture of 2-NBDG and D-glucose in HFD mice treated 4 weeks with i.p injections of FcsFNDC4 or VC. Fluorescence was normalized to tissue autofluorescence and weight. I) Western blot of pAKT (Ser473), total AKT and VCP (loading control) from lysates of indicated tissues derived from HFD mice (16 weeks on HFD 60% fat) treated for 4 weeks with VC or FcsFNDC4. Mice were fasted overnight (12-16 h), 5 U/kg insulin (+) or saline (−) was i.p. injected and 8 min later indicated organs were excised and snapped frozen in liquid nitrogen. j) Mean cell size of adipocytes from gWAT, measured on fixed paraffin section stained with H&E staining. k) % of Cd68 positive area per tissue (gWAT). l) (m-left) Hematoxylin eosin (H&E) staining of gWAT in VC and FcFndc4 injected mice. Bars=200 μm. (m-right) Immunohistochemical detection of CD68 in gWAT of VC and FcsFndc4 mice. Paraffin sections. Chromogene: 3,3′-Diaminobenzidine (DAB, brown color); nuclear counterstain: Hemalaun. Bars=200 μm. m RT-qPCR quantification of Cd68 mRNA levels in total gWAT. l, n) RT-qPCR of indicated genes in gWAT. o) ELISA quantification of plasma TNFalpha levels (pg/ml). p) ELISA quantification of serum resistin levels (ng/ml). During IPGTT and glucose induced insulin test 2 g/kg D-glucose was injected and during the ITT 0.8 U/kg insulin was used. Mice were C57BL6N males, n=6-7 per group. Data shown are mean+SEM. (*) indicates p<0.05, (**) indicates p<0.01, (***) indicates p<0.001 using Student's t-test, ns; non-significant. One-way ANOVA was applied to assess the time effect in pzt.

FIG. 6: Identification of GPR116 as a candidate receptor for sFNDC4. a) Saturation binding curves of FcsFNDC4 and Fc control to immortalized SVF preadipocytes derived from mouse iWAT. Y-axis shows ligand binding as mean fluorescence intensity (MFI) per 10000 cells. b) Competition of FcsFNDC4 binding (500 nM) with increasing concentrations of untagged sFNDC4. Data are expressed as % of max binding of 500 nM FcsFNDC4. The mean+SEM of 3 technical replicates (triplicates) is shown. These experiments were repeated 3 times. c) Sorted cell populations of imm. SVF iWAT to very high log. PE/log.FITC signal (top 2.25%=HBC) versus very low log.PE/FITC signal (bottom 3.92%=LBC), positive for FcsFNDC4 binding (100 nM). d) Binding of FcsFNDC4 (20 nM) and Fc control (20 nM) or only IgG-PE secondary in cells sorted from c after seeding for 20 passages. Bars correspond to binding shown as MIF per 10000 cells. This experiment was performed several times up to passage 20. Mean of three technical replicates is shown. e) mRNA expression heat map of the top 35 genes identified with Fold change HBC vs LBC >1.5 and p<0.05 from Affymetrix gene expression arrays comparing LBC and HBC. Receptor genes are underlined. Color scale of fold change values is shown below the heatmap. f) Fold change of binding (MFI) per 10000 cells of FcsFNDC4 (100 nM) binding to HEK293 cells with transient overexpression of human RXFP1 or human GPR116 or human ITGAD. Binding to a mock transfection control (lipofectamin only) was used as control. g) (left and right panel) qPCR quantification of indicated genes at indicated conditions. h) Competition of FcsFNDC4 (500 nM) binding with increasing concentrations of EDTA (0-103 μM). Values are binding shown as MFI per 10000 cells. Bars represent mean two experiments with three technical replicates. This experiment was performed two times. i) Binding (MFI) of indicated concentrations of FcsFNDC4 of Fc control to SVF derived mouse primary preadipocytes from WT, GPR116+/− and GPR116−/− mice. This experiment was performed once and iWAT SVF cells were pooled from 7-8 mice. Mice were male, 7-9 weeks old. Single data point for binding is shown for each tested concentration of rec. protein. (i-top panel) mRNA levels of GPR116 in SVF iWAT preadipocytes used for binding in i at indicated genotypes. j Pull down for GPR116: Western blot analysis against GPR116. For pull down 6×His Dynabeads were prebound with 6×His-Fc, 6×His-Fc-sFNDC4 (30 μg), followed by incubation 300 μg of NIH3T3 cell lysates. (+) added to beads, (−) not added to beads, Input: rec. protein or cell lysates added to the Dynabeads. Unretained: Cell lysates proteins that did not immunoprecipitate to the beads. Eluted: Elution of GPR116, which precipitated on 6×His-Fc-fused protein prebound to beads. Similar results were obtained from three independent experiments. Antibody against GPR116: ab136262. k Mean fluorescence intensity (PE) representing binding of FcsFNDC4 (100 nM) or Fc control (100 nM) to HEK293 GPR116 (human) OE cells in the presence of indicated dose of antiGPR116 antibody, against the N-terminus of GPR116 (ab111169) or isotype control (ab171870). This experiment was performed once with three technical replicates. I q-PCR quantification of human Gpr116 mRNA levels in HEK293A and HEK293T cells. m, n Saturation binding on HEK293A (m) or HEK293T (n) cells with stable human GPR116 OE or mock cells after incubation with indicated concentrations of FcsFNDC4 or Fc control. For n FcsFNDC4 binding was also performed in excess of sFNDC4 (1 mM) to determine non-specific binding (NSB). Calculated equilibrium binding constant (Kd) is shown for total binding in m and specific binding in n. Y-axis shows binding shown as MIF mean+SEM of three technical replicates. These experiments were performed 3 times. I Data are shown as mean+SEM. Statistical analysis is a Student's t-test (*) p-value<0.05, (**) p-value<0.01. Student's t-test, ns; non-significant. For statistical analysis of Affymetrix, data see methods. Error bars represent standard error of the mean (SEM).

FIG. 7: HFD fed GPR116^{Ad-/-} mice did not improve glucose tolerance in response to FcsFNDC4 therapeutic injections as opposed to GPR116^{Adf}/f mice. a) Blood glucose during IPGTT test at indicated time points and area under the curve b). c) Glucose stimulated insulin response during the IPGTT in (a and b). d) Blood glucose as % of baseline glucose levels during an ITT. e) Body weight, f) organ weight, g) Serum resistin and h) plasma TNFalpha at indicated groups. White bars: GPR116^{Adf/f} Fc injected mice, red bars: GPR116^{Adf/f} FcsFNDC4 injected mice, black bars GPR116^{Ad-/-}Fc injected mice, red bars/pattern: GPR116^{Ad-/-} FcsFNDC4 injected. Mice were males, set on a HFD 60% fat, 9-10 weeks old for 12 weeks. FcsFNDC4 injections were given at a dose of 0.2 mg/kg every second day for 4 weeks (injections between weeks 8-12 of HFD), n=6-8 per group. During IPGTT and glucose induced insulin test 2 g/kg D-glucose was injected and during the ITT 0.8 U/kg insulin was used. Bars are means+SEM. Statistics represents Student's t-test. (*) p-value<0.05, (**) p-value<0.01. Student's t-test, ns; non-significant.

FIG. 8: sFNDC4 insulin sensitizing effects in 3T3L1 adipocytes require interraction with GPR116 and involve Gs-cAMP signaling. a) WB of indicated proteins: Overnight incubation (O/N-16 h) of 3T3L1 adipocytes with FcsFNDC4 (FcsF4) or Fc with 10 nM of insulin or without insulin (w/o). Following O/N incubations the cells were serum starved for 3 h. After that cells were stimulated with insulin at indicated concentrations (0 nM, 0.5 nM, 1 nM) for 5 min. b) WB of indicated proteins: 3T3L1 adipocytes were treated overnight with insulin (10 nM) or w/o and FcsFNDC4 or Fc in the presence of antiGPR116 (ab111169) (0.4 μg/ml) or isotype control (0.4 μg/ml). Prior acute insulin stimulation for 5 min, cells were incubated in serum free media (SFM) for 3 h. c) Tritium labelled glucose uptake at indicated conditions. Cells were treated as in b) and after serum starvation were stimulated with 1 nM insulin for 20 min and glucose transport was initiated by the addition of [3H]2-deoxyglucose (PerkinElmer Life Sciences) (0.25 μCi/well, 50 μM unlabeled 2-deoxyglucose) for 5 min, when the experiment was terminated. d), e) Stimulation of 3T3L1 adipocytes luciferase reporter stable cells lines with indicated concentrations of stimuli. Stimulation was 3-4 h for the CRE-, SRE- and SRF-luc2P cell lines and 16 h for the NFAT-RE luc2P cell line. For e) antiGPR116 (ab111169) 0.4 ug/ml was added 30 min prior the addition of the rec. proteins. Media was removed and replaces with media containing indicated concentrations of rec. proteins. F) WB: 3T3L1 adipocytes and g) WB: adipocytes derived from mouse primary SVF cells were incubated in SFM for 3 h and then stimulated in SMF with indicated dose of rec. protein or antiGPR116 antibody 0.4 ug/ml for g) and for indicated duration of incubation (min). is a pool of 2 independent experiments. d), f), g) was performed several times, e) was performed once under the exact shown conditions. In d), e) phosphodiesterase (PDE) inhibitor, IBMX (0.5 mM) was present in the media during the treatment of the Cre2LucP adipocytes, whereas in f), g) no phosphodiesterase (PDE) inhibitors were present. Bars are means±SEM. Statistics represents Student's t-test. (*) p-value<0.05, (**) p-value<0.01, (***) p-value<0.001, (****) p-value<0.0001 Student's t-test, ns; non-significant, NT; non treated. Phospho-antibodies: pAKT^{Ser473}, pCREB^{Ser133}

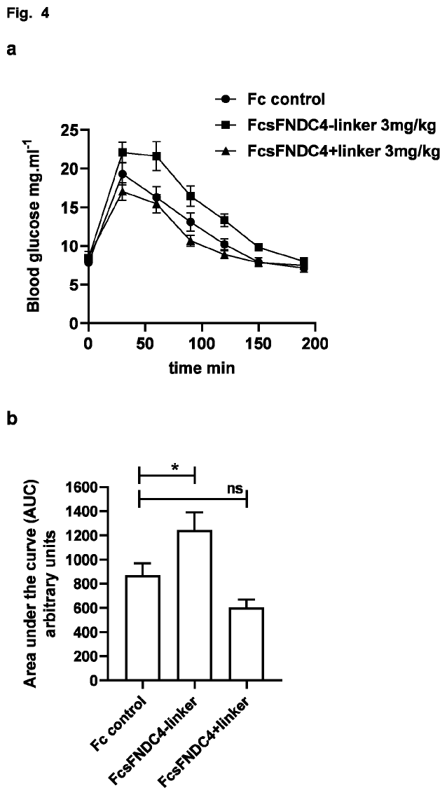

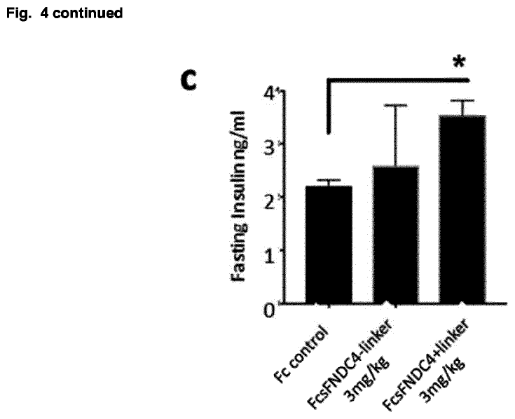

FIG. 9: Comparison of high and low dose of FcsFNDC4 of the invention (also mentioned elsewhere in the present document, including FIG. 4 as “FcsFNDC4+linker” (plus linker)) on 8 weeks HFD mice. Blood glucose levels during an IPGTT where 2 g/kg D-glucose was injected after 6 hours fasting. Groups are mice injected for two weeks i.p either with a high dose of FcsFNDC4 (FcsFNDC4+linker) 3 mg/kg, a low dose of FcsFNDC4 (FcsFNDC4+linker) or a vehicle control (PBS).

FIG. 10: Elisa quantification of human IgG1 in mouse plasma at indicated time points pose injection. a) FcsFNDC4, 1 mg/kg was injected subcutaneously (SC), n=3 mice per time point. b) FcsFNDC4, 1 mg/kg was injected IV, n=3 mice per time point. Data shown is mean+SEM.

### DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments described throughout the specification should not be construed to limit the present invention to only the explicitly described embodiments. This description should be understood to support and encompass embodiments which combine the explicitly described embodiments with any number of the disclosed and/or preferred elements. Furthermore, any permutations and combinations of all elements described herein should be considered disclosed by the description of the present application unless the context indicates otherwise.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e. the subject-matter consists in the inclusion of a stated member, integer or step or group of members, integers or steps. When used herein the term “comprising” can be substituted with the term “containing” or “including” or sometimes when used herein with the term “having”. When used herein “consisting of” excludes any element, step, or ingredient not specified.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Unless otherwise indicated, the term “at least” preceding a series of elements is to be understood to refer to every element in the series. The term “at least one” refers to one, two, three or more such as four, five, six, seven, eight, nine, ten and more. Those skilled in the art will recognize, or be able to ascertain using no more than routine experimentation, many equivalents to the specific embodiments of the invention described herein. Such equivalents are intended to be encompassed by the present invention.

The term “less than” or in turn “more than” or “below” does not include the concrete number.

The term “and/or” wherever used herein includes the meaning of “and”, “or” and “all or any other combination of the elements connected by said term”.

When used herein “consisting of” excludes any element, step, or ingredient not specified in the claim element. When used herein, “consisting essentially of” does not exclude materials or steps that do not materially affect the basic and novel characteristics of the claim.

The term “including” means “including but not limited to”. “Including” and “including but not limited to” are used interchangeably.

The term “about” means plus or minus 20%, preferably plus or minus 10%, more preferably plus or minus 5%, most preferably plus or minus 1%.

Throughout the description and claims of this specification, the singular encompasses the plural unless the context otherwise requires. In particular, where the indefinite article is used, the specification is to be understood as contemplating plurality as well as singularity, unless the context requires otherwise.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

A better understanding of the present invention and of its advantages will be gained from the examples, offered for illustrative purposes only. The examples are not intended to limit the scope of the present invention in any way.

### Fusion Protein

Accordingly, the present invention relates to a fusion protein comprising at least three subunits. The term “polypeptide” can be used interchangeably with the term “protein” as used in the present invention. Preferably, the fusion protein is a translational fusion between the three subunits. The translational fusion may be generated by genetically engineering the coding sequence for one subunit in frame with the coding sequence of the other two subunits. In particular, the fusion protein according to the invention comprises a) a soluble FNDC4 (sFNDC4) or a functional fragment thereof; b) a peptide linker; and c) a Fc-domain. The subunits as described may refer to the sFNDC4, the linker and the Fc-domain or any other subunit, for example the subunits may refer to chemical subunits, such as chemical subunits which extend the half-life of the protein in the circulation, such as bulking moieties for example polyethylene glycol, O- and N-linked oligosaccharides, dextran, hydroxyethyl starch (HES), polysialic acid and hyaluronic acid, as well as unstructured protein polymers such as homo-amino acid polymers, elastin-like polypeptides, XTEN and PAS or conjugation with fatty acids or albumin or transferrin being comprised by the fusion protein. Such fusion protein thus refers to the term “Fc-sFNDC4” or “FcsFNDC4” as it is used herein. Said fusion protein called “Fc-sFNDC4” comprises the abovementioned three subunits a)-c), but may also comprise any other subunit as defined herein. According to the present invention, such fusion protein is recombinant.

The term “FNDC4” as used throughout the present invention refers to fibronectin type Ill domain containing 4. It is a type I transmembrane protein. Such FNDC4 has been already demonstrated to release a soluble bioactive protein that is highly conserved amongst mouse and primates Bosma, M., et al. (2016), Nat. Commun. 7). In the present invention said FNDC4 protein or functional fragment thereof is thus also soluble, which refers to sFNDC4. In particular, in the context of the present invention the term “soluble FNDC4” or “sFNDC4” refers to the extra-cellular portion/part/domain of wild type full length FNDC4 which is released from the transmbembrane domain by proteolytic cleavage. In this context, the term “soluble” means thus generally soluble in water or aqueous media. In particular, soluble proteins may be proteins which are found free in cellular compartments such as the cytoplasm, nucleus or endoplasmic reticulum. So far, soluble FNDC4 (sFNDC4) has been reported in the prior art to exert anti-inflammatory effects on macrophages and osteoclasts promoting survival in response to severe chronic inflammation (Bosma, M., et al. (2016). Nat. Commun. 7). Such sFNDC4 has a C-terminal residue at the C-terminus and a N-terminal residue at the N-terminus as it is known to a person skilled in the art.

In particular, the soluble FNDC4 of the fusion protein as described herein may comprise an amino acid sequence having at least about 70% identity, at least about 75%, at least about 80%, at least about 85%, at least about 90%, at least about 95%, including at least about 96%, 97%, 98%, 99% or even 100% sequence identity with the amino acid sequence of with an amino acid sequence of SEQ ID NO.: 1. Preferably, said sFNDC4 or a functional fragment thereof as described herein originates from human or mouse. Even more preferably, the sFNDC4 is a human soluble FNDC4 having the amino acid sequence depicted in SEQ ID NO.: 1 or a functional fragment thereof. SEQ ID NO.: 1 corresponds here to the soluble portion (extra-cellular portion) as defined elsewhere herein of the protein having the Uniprot accession number Q9H6D8, which is the native form of the full-length human FNDC4 (hFNDC4). Alternatively, the sFNDC4 of the fusion protein as described herein may comprise the amino acid sequence sequence having at least about 70% identity, at least about 75%, at least about 80%, at least about 85%, at least about 90%, at least about 95%, including at least about 96%, 97%, 98%, 99% or even 100% sequence identity with the amino acid sequence as depicted in SEQ ID NO.: 2. Also preferred is that the sFNDC4 is a mouse soluble FNDC4 having the amino acid sequence as depicted in SEQ ID NO.: 2 or a functional fragment thereof. SEQ ID NO.: 2 corresponds here to the soluble portion (extra-cellular portion) as defined elsewhere herein of the protein having the Uniprot accession number Q3TR08, which is the native form of mouse full-length FNDC4 (mFNDC4). In these embodiments, the term “at least about” includes each single %-value starting from 70% to 100% sequence identity, such as at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or even 100% sequence identity with an amino acid sequence of SEQ ID NO.: 1.

By “identity” or “sequence identity” is meant a property of sequences that measures their similarity or relationship. The term “sequence identity” or “identity” as used in the present invention means the percentage of pair-wise identical residues—following (homology) alignment of a sequence of a polypeptide of the invention with a sequence in question—with respect to the number of residues in the longer of these two sequences. Identity is measured by dividing the number of identical residues by the total number of residues and multiplying the product by 100. The percentage of sequence identity can, for example, be determined herein using the program BLASTP, version blastp 2.2.5 (Nov. 16, 2002; cf. Altschul, S. F. et al. (1997) Nucl. Acids Res. 25, 3389-3402). In this embodiment the percentage of homology is based on the alignment of the entire polypeptide sequences (matrix: BLOSUM 62; gap costs: 11.1; cutoff value set to 10-3) including the respective sequences. It is calculated as the percentage of numbers of “positives” (homologous amino acids) indicated as result in the BLASTP program output divided by the total number of amino acids selected by the program for the alignment.

Any types and numbers of mutations, including substitutions, deletions, and insertions are thus envisaged as long as a provided fusion protein retains its capability to bind its given ligand/target, such as GPR116, and/or it has a sequence identity that it is at least about 70%, at least about 75%, at least about 80%, at least about 85%, at least about 90%, at least about 95% or higher identity to the amino acid sequence of SEQ ID NO.: 1.

Accordingly, a functional fragment of sFNDC4 as used in the context of the present invention refers to a functional equivalent having the same functional characteristics as the sFNDC4 protein as defined herein. Hence, said functional fragment, no matter what length it has, still comprises the adhesion G-protein-couple receptor 116 (GPR116) binding domain. Thus, also a fusion protein as defined herein comprising a functional fragment of sFNDC4 can be understood as a fusion protein in the context of the present invention. In particular, the functional fragment of sFNDC4 may be at least about 10 amino acids long, preferably at least about 12 amino acids, preferably at least about 20 amino acids, preferably at least about 25 amino acids, preferably at least about 30 amino acids, preferably at least about 35 amino acids, or even preferably up to about 90 amino acids long. More preferably, said functional fragment of sFNDC4 is between about 90 amino acids to about 10 amino acids long, such as between about 85 amino acids to about 15 amino acids long, between about 80 amino acids to about 20 amino acids long, between about 75 amino acids to about 25 amino acids long, between about 70 amino acids to about 30 amino acids long, even more preferably between about 70 amino acids to about 35 amino acids long, even more preferably between about 65 amino acids to about 35 amino acids long, even more preferably between about 65 amino acids to about 40 amino acids long, even more preferably between about 60 amino acids to about 40 amino acids long, even more preferably between about 62 amino acids to about 50 amino acids long.

The fusion protein according to the present invention as described above further comprises a peptide linker as one of the three subunits mentioned elsewhere herein. A “linker” that may be comprised by said fusion protein of the present disclosure links two or more subunit(s) of said fusion protein as described herein. The linkage can be covalent or non-covalent. Preferably, said linkage is covalently. Such preferred covalent linkage is via a peptide bond, such as a peptide bond between amino acids. A preferred peptide linker as described herein comprises between about 5 and about 13 amino acids, such as 5, 6, 7, 8, 9, 10, 11, 12 or 13 amino acids, preferably about 9 amino acids. It is preferred that the linker molecule is a linear or a helical linker, even more preferably the linker is a helical linker. It is further preferred that the linker is a flexible linker using e.g. the amino acids glycine and/or serine.

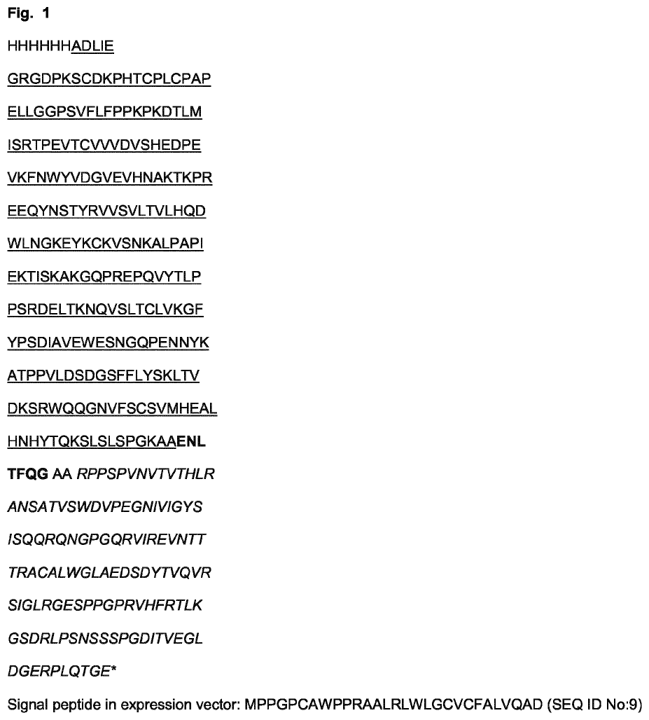

Preferably, the C-terminal residue of the peptide linker is directly fused to the N-terminus of the sFNDC4 of said fusion protein of the present invention. The term “directly fused” means that said linker and said N-terminus of the sFNDC4 are arranged one after the other without using any linker. When the linker is directly fused to the N-terminus of the sFNDC4, said Fc-domain as third a subunit of the fusion protein can either be fused to the N-terminus (N-terminal residue) of said linker or to the C-terminus (C-terminal residue) of said sFNDC4. Even more preferably, the N-terminal residue of the peptide linker is directly fused to the C-terminal residue of said Fc-domain described elsewhere herein. In other words, said C-terminal residue of the Fc-domain as a third subunit of the fusion protein is directly fused to the N-terminal residue of said linker. For this particular embodiment, said peptide linker is arranged inbetween said Fc-domain and said sFNDC4 as can be seen in FIG. 1.

A preferred peptide linker of the fusion protein of the present invention comprises a Tobacco Etch Virus (TEV) protease site. Such site can be understood as a recognition sequence/site for said highly sequence-specific cysteine protease from Tobacco Etch Virus (TEV). In other words, said peptide linker comprises a recognition sequence/site for TEV protease. In general, such sequence/site may be used that said TEV protease can recognize its target and will then be able to modify it enzymatically. As it is known to a person skilled in the art, such protease recognizes the amino acid sequence Glu-Asn-Leu-X-Phe-Gln-X (SEQ ID NO: 8) and cleaves between the Gln and the residue X at position 7 corresponding to SEQ ID NO: 8. SEQ ID NO: 8 comprises at position 4 corresponding to SEQ ID NO: 8 tyrosine (Tyr; Y) or threonine (Thr; T) and/or at position 7 corresponding to SEQ ID NO: 8 a glycine (Gly; G) or a serine (Ser; S). Preferably, the TEV site comprised by said linker comprises at position 4 corresponding to SEQ ID NO: 8 a threonine and/or at position 7 corresponding to SEQ ID NO: 8 a glycine. Said TEV site being comprised in said peptide linker of the fusion protein of the present invention preferably refers to E-N-L-T-F-Q-G as can be seen in FIG. 1.

In a most preferred embodiment, the peptide linker of the fusion protein comprises the amino acid sequence of SEQ ID NO.: 3. Said particular amino acid sequence of the peptide linker as it is depicted in SEQ ID NO.: 3 comprises at position 8 and 9 any amino acid (see Table 1).

As mentioned above, the fusion protein according to the present invention additionally comprises a Fc domain as the thir subunit. The term “Fc domain” or “Fc fragment” is used herein to define a C-terminal region of an immunoglobulin heavy chain, including native-sequence Fc regions and variant Fc regions. The Fc part mediates the effector function of fused proteins, including antibodies, e.g. the activation of the complement system and of Fc-receptor bearing immune effector cells, such as NK cells. In human IgG molecules, the Fc region is generated by papain cleavage N-terminal to Cys226. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody molecule, or by recombinantly engineering the nucleic acid encoding a heavy chain of the antibody molecule.

Preferable native-sequence Fc-domains comprised by the fusion protein of the invention include mammalian, e.g. human or murine, IgG1, IgG2 (IgG2A, IgG2B), IgG3 and IgG4. The Fc-domain contains two or three constant domains, depending on the class of the antibody. In embodiments where the immunoglobulin is an IgG, the Fc-domain has a CH2 and a CH3 domain. Preferably and as already mentioned elsewhere herein, the C-terminal residue of said Fc-domain as defined herein is directly fused to the N-terminal residue of the peptide linker as defined elsewhere herein. Most preferably, the Fc-domain of the fusion protein of the present invention is an IgG1 Fc-domain, such as a human or a mouse IgG1 Fc-domain.

In respect to the fusion protein (FcsFNDC4) of the present invention, the term “comprising” denotes that further components/subunits or molecules can be included in addition to the specifically recited components/subunits (i.e. a sFNDC4 or functional fragment thereof, a peptide linker and a Fc-domain) such as labels or tags as described elsewhere herein. For example, in those embodiments where the FcsFNDC4 of the present invention includes more than the recited components, said additional molecules, preferably at the C- or N-terminus of the FcsFNDC4, may include for example sequences introduced for purification, typically peptide sequences that confer on the resulting FcsFNDC4 an affinity to certain chromatography column materials. Typical examples for such sequences include, without being limiting, tags such as an oligohistidine-tag, a Sirep-tag, a FLAG-tag, a histidine tag, a glutathione S-transferase (such as GST or GST-SUMO3 tag), a maltose-binding protein or the albumin-binding domain of protein G. In preferred embodiments, the FCsFNDC4 further comprises a 6×His-tag, preferably the 6×His-tag is located at the N-terminus of the FcsFNDC4.

In a most preferred embodiment, the present invention relates to a fusion protein as defined elsewhere herein, having the amino acid sequence of SEQ ID NO.: 4, which refers to the whole sequence of said fusion protein comprising hsFNDC4 as depicted by SEQ ID NO.: 1, said peptide linker as depicted by SEQ ID NO.: 3 and said IgG1 Fc-domain as depicted by SEQ ID NO.: 6. Also comprised herein, is said fusion protein comprising an amino acid sequence having at least about 60%, at least about 65%, at least about 70%, at least about 75%, at least about 80%, at least about 85%, at least about 90%, at least about 95%, including at least about 96%, 97%, 98%, 99% or even 100% sequence identity with an amino acid sequence of SEQ ID NO.: 4.

Also comprised by the present invention and thus also mostly preferred is a fusion protein as defined elsewhere herein, having the amino acid sequence of SEQ ID NO.: 5, which refers to the whole sequence of said fusion protein comprising msFNDC4 as depicted by SEQ ID NO.: 2, said peptide linker as depicted by SEQ ID NO.: 3 and said IgG1 Fc-domain as depicted by SEQ ID NO.: 7. Also comprised herein, is said fusion protein comprising an amino acid sequence having at least about 60%, at least about 65%, at least about 70%, at least about 75%, at least about 80%, at least about 85%, at least about 90%, at least about 95%, including at least about 96%, 97%, 98%, 99% or even 100% sequence identity with an amino acid sequence of SEQ ID NO.: 5.

In the context of the present invention, the fusion protein preferably has binding affinity to the orphan adhesion G protein couple receptor (GPCR) 116 (GPR116). The identification of said particular sFNDC4 receptor is a key information with respect to the in vivo mode of action of sFNDC4. The inventors identified the up to date orphan GPR116 to be a functional receptor of FcsFNDC4 in vitro and in vivo (FIG. 6). GPR116 is a typical member of the adhesion GPCRs family, which are hybrids. They possess a long extracellular N-terminal fragment (NTF), which is proteolytically cleaved, leaving the remaining C-terminal fraction of the receptor (CTF) attached to the cell membrane. NTF can modulate the baseline activity of adhesion GPCRs by a) parts of it being non-covalently associated with the extracellular interface of the 7TM part of the remaining GPCR, b) by interacting with other adjacent membrane or extracellular matrix proteins (Langenhan, T., Aust, G., and Hamann, J. (2013), Sci. Signal. 6, re3). Signaling via the NTF of adhesion GPCRs is complex and not well understood. Although CTF, like in canonical GPCRs, initiates interactions with heterotrimeric G-proteins, NTF signaling has been shown in certain cases to act autonomously or interplays with the CTF to initiate or not heterotrimeric G protein signaling (Prömel, S., et al. (2012)). Due to these properties, NTF may provide spatiotemporal and context specific signaling properties to its receptor.

In particular, the inventors surprisingly identified the GPR116 as a receptor of sFNDC4 in white adipose tissue (WAT), thereby establishing a novel endocrine FNDC4-GPR116 axis in the control of systemic glucose homeostasis. Intriguingly, this axis was impaired in diabetic patients and therapeutic injections of recombinant FcsFNDC4 into diabetic mice corrected diabetic hyperglycemia, providing a rationale for harnessing the FNDC4-GPR116 axis in diabetes therapy (see FIGS. 6-8).

As used herein, “binding affinity” of a fusion protein of the present invention (e.g. FcsFNDC4) to a selected ligand (in the present case GPR116) can be measured by a multitude of methods known to those skilled in the art. Such methods include, but are not limited to, fluorescence titration, competition ELISA, calorimetric methods, such as isothermal titration calorimetry (ITC) and surface plasmon resonance (BIAcore). Such methods are well established in the art and examples thereof are also detailed below. It is also noted that the complex formation between the respective fusion protein and its ligand is influenced by many different factors such as the concentrations of the respective binding partners, the presence of competitors, pH and the ionic strength of the buffer system used, and the experimental method used for determination of the dissociation constant K_{D} (for example fluorescence titration, competition ELISA or surface plasmon resonance, just to name a few) or even the mathematical algorithm which is used for evaluation of the experimental data. Therefore, it is also clear to the skilled person that the K_{D} values (dissociation constant of the complex formed between the respective fusion protein and its ligand) may vary within a certain experimental range, depending on the method and experimental setup that is used for determining the affinity of said particular fusion protein for a given ligand, such as GPR116. This means that there may be a slight deviation in the measured K_{D} values or a tolerance range depending, for example, on whether the K_{D} value was determined by surface plasmon resonance (Biacore), by competition ELISA, or by “direct ELISA.”

In a more preferred embodiment, the fusion protein of the present invention may specifically bind to the N-terminus of the GPR116 receptor. In this context, this means that the fusion protein of the present invention may specifically bind to the extracellular N-terminal fragment (NTF) of GPR116 as defined elsewhere herein, which is proteolytically cleaved, leaving the remaining C-terminal fraction of the receptor (CTF) attached to the cell membrane. The term “specifically binds” generally indicates that said fusion protein of the present invention binds with higher affinity to its intended ligand/target (i.e. the GPR116 receptor as described herein) than to its non-target/non-ligand molecule. Preferably the affinity of the fusion protein will be at least about 5 fold, preferably 10 fold, more preferably 25-fold, even more preferably 50-fold, and most preferably 100-fold or more, greater for said target, the GPR116 receptor as described herein than its affinity for a non-target molecule. Preferably, the term “specifically binds” thus indicates that said fusion protein of the present invention exclusively binds to its intended target (i.e., the GPR116 receptor as described herein).

In some embodiments, the present invention also relates to a fusion protein comprising a) a soluble FNDC4 (sFNDC4) or a functional fragment thereof; b) a peptide linker; and c) a Fc-domain as defined elsewhere herein, wherein the fusion protein is administered to the subject as defined elsewhere herein in a dosage below 3 mg/kg according to the present invention.

### Nucleic Acid Molecules

Further comprised by the present invention, is a nucleic acid molecule comprising a nucleotide sequence encoding said fusion protein as defined elsewhere herein.

Nucleic acid molecules comprising a nucleotide sequence encoding said fusion protein include DNA, such as cDNA or genomic DNA, and RNA. Preferably, embodiments reciting “RNA” are directed to mRNA.

The present invention also relates to nucleic acid molecules as defined herein comprising nucleotide sequences coding for said fusion protein as described herein. Since the degeneracy of the genetic code permits substitutions of certain codons by other codons specifying the same amino acid, the invention is not limited to a specific nucleic acid molecule encoding said fusion protein of the invention but includes all nucleic acid molecules comprising nucleotide sequences encoding a functional fusion protein.

In some embodiments, a nucleic acid molecule comprising a nucleotide sequence encoding said fusion protein disclosed in this application, such as DNA, may comprise nucleotide sequences which are “operably linked” to one another. i.e a nucleotide sequence encoding for said soluble FNDC4 as defined elsewhere herein, a nucleotide sequence encoding for said linker as defined elsewhere herein, and a nucleotide sequence encoding for said Fc domain as defined elsewhere herein. Said nucleotide sequences are operably linked to one another. In this regard, an operable linkage is a linkage in which the sequence elements of one nucleotide sequence and the sequence elements of another nucleotide sequence are connected in a way that enables expression of the fusion protein as a single protein.

The invention also includes nucleic acid molecules encoding the fusion protein as described elsewhere herein, which include additional mutations outside the indicated sequence positions of experimental mutagenesis. Such mutations are often tolerated or can even prove to be advantageous, for example if they contribute to an improved folding efficiency, serum stability, thermal stability or ligand binding affinity of the fusion protein.

A nucleic acid molecule disclosed in this application may be “operably linked” to a regulatory sequence (or regulatory sequences) to allow expression of this nucleic acid molecule.

A nucleic acid molecule, such as DNA, is referred to as “capable of expressing a nucleic acid molecule” or capable “to allow expression of a nucleotide sequence” if it includes sequence elements which contain information regarding to transcriptional and/or translational regulation, and such sequences are “operably linked” to the nucleotide sequence encoding the fusion protein. An operable linkage is a linkage in which the regulatory sequence elements and the sequence to be expressed are connected in a way that enables gene expression. The precise nature of the regulatory regions necessary for gene expression may vary among species, but in general these regions include a promoter which, in prokaryotes, contains both the promoter per se, i.e. DNA elements directing the initiation of transcription, as well as DNA elements which, when transcribed into RNA, will signal the initiation of translation. Such promoter regions normally include 5′ non-coding sequences involved in initiation of transcription and translation, such as the −35/−10 boxes and the Shine-Dalgarno element in prokaryotes or the TATA box, CAAT sequences, and 5-capping elements in eukaryotes. These regions can also include enhancer or repressor elements as well as translated signal and leader sequences for targeting the native polypeptide to a specific compartment of a host cell.

In addition, the 3′ non-coding sequences may contain regulatory elements involved in transcriptional termination, polyadenylation or the like. If, however, these termination sequences are not satisfactory functional in a particular host cell, then they may be substituted with signals functional in that cell.

Therefore, a nucleic acid molecule of the present invention can include a regulatory sequence, such as a promoter sequence. In some embodiments a nucleic acid molecule of the present invention includes a promoter sequence and a transcriptional termination sequence. Suitable prokaryotic promoters are, for example, the tet promoter, the lacUV5 promoter or the T7 promoter. Examples of promoters useful for expression in eukaryotic cells are the SV40 promoter or the CMV promoter.

### Vector

The nucleic acid molecules of the present invention can also be part of a vector or any other kind of cloning vehicle, such as a plasmid, a phagemid, a phage, a baculovirus, a cosmid or an artificial chromosome, preferably part of a vector.

Such cloning vehicles can include, aside from the regulatory sequences described above and a nucleic acid molecule comprising a nucleotide sequence encoding said fusion protein as described herein, replication and control sequences derived from a species compatible with the host cell that is used for expression as well as selection markers conferring a selectable phenotype on transformed or transfected cells. Large numbers of suitable cloning vectors are known in the art, and are commercially available. The vector may also comprise a signal peptide, preferably the signal peptide of SEQ ID NO: 9 (see FIG. 1).

The nucleic acid molecule encoding said fusion protein as described herein (for example the fusion protein of SEQ ID NOs: 4 or 5), and in particular a cloning vector containing the coding sequence of such a fusion protein of the invention can be transformed into a host cell capable of expressing the gene. Transformation can be performed using standard techniques (Sambrook, J. et al. (1988) Molecular Cloning: A Laboratory Manual, 2nd Ed).

### Host Cell

Thus, the present invention is also directed to a host cell comprising said nucleic acid molecule or said vector as disclosed herein.

The transformed host cells are cultured under conditions suitable for expression of the nucleotide sequence encoding said fusion protein of the invention. Suitable host cells can be prokaryotic, such as Escherichia coli (E. coli) or Bacillus subtilis, or eukaryotic, such as Saccharomyces cerevisiae, Pichia pastoris, SF9 or High5 insect cells, immortalized mammalian cell lines such as HeLa cells or CHO cells or primary mammalian cells, preferably CHO-S cells.

### In Vivo Therapeutic Applications

The present invention further refers to the fusion protein as described elsewhere herein or a composition comprising such fusion protein for use as a medicament. Hence, the fusion protein as described elsewhere herein of the present invention or a composition comprising such fusion protein can also be used for therapy, i.e. the treatment of a disease associated with glucose intolerance and/or insulin resistance and/or impaired insulin production. Accordingly, the present invention relates to a fusion protein as described elsewhere herein for use in a method of preventing and/or treating diabetes in a subject.

The term “diabetes” as used herein refers to a group of metabolic disorders characterized by impaired insulin production, insulin resistance and/or impaired glucose tolerance (=glucose intolerance) resulting in a high blood sugar level. Diabetes results from a deficiency or functional impairment of insulin-producing p cells, alone or in combination with insulin resistance. If left untreated, diabetes can cause many complications. Diabetes is thus due to either the pancreas not producing enough insulin, or the cells of the body not responding properly to the insulin produced. As it is known to the skilled artisan there are three main types of diabetes: Type 1 diabetes (T1D) results from the pancreas's failure to produce enough insulin due to loss of β-cells. T1D is also known as Insulin Dependent Diabetes Mellitus (IDDM) and juvenile diabetes. The terms are used interchangeably herein. This form accounts for 5-10% of diabetes and as mentioned is thought to be due to cellular-mediated autoimmune destruction of the pancreatic β-cells, resulting in little or no insulin secretion. Type 2 diabetes (T2D) also refers to as adult-onset diabetes and accounts for ˜90-95% of all diabetes. Besides glucose intolerance, insulin resistance in target tissues and a relative deficiency of insulin secretion from pancreatic β-cells are the major features of T2D. Insulin resistance is used herein to denote a condition characterized by the failure of target cells to respond to insulin, leading to hyperglycemia. Pancreatic β cells in the pancreas subsequently increase their production of insulin, leading to hyperinsulinemia. The most common cause is a combination of excessive body weight and insufficient exercise. Gestational diabetes is the third main form and occurs when pregnant women without a previous history of diabetes develop high blood sugar levels. Defective insulin secretion underlies all forms of diabetes mellitus. Whereas the destruction of β-cells is responsible for T1 D, both lowered β-cell mass and loss of secretory function are implicated in T2D. Emerging results suggest that a functional deficiency, involving de-differentiation of the mature β-cell towards a more progenitor-like state, may be an important driver for impaired secretion in T2D. T2D also involves mild chronic inflammation.

The term “β cell(s)”, “beta cell(s)” and “islet cell(s)” are used interchangeably herein to refer to the pancreatic β cells located in the islet of Langerhans. Their primary function is to store and release insulin.

As it is known to the person skilled in the art, when a subject suffers from prediabetes, the pancreas does not make enough insulin or cells become resistant to insulin. Thus, elevated fasting blood glucose (IFG) or impaired glucose tolerance (IGT) is already present, but manifest diabetes is yet not present. Prediabetes is more accurately considered an early stage of diabetes as health complications associated with type 2 diabetes often occur.

The term “glucose intolerance” thus also refers to a hallmark of pre-diabetic state (pre-diabetes), and it is characterized by the inability to remove excess glucose from the blood circulation which can then lead to obesity-related T2D. Progressive insulin resistance and the subsequent failure to cope with dietary glucose, i.e. glucose intolerance, mostly reflects the inability of adipose tissue and skeletal muscle to sufficiently eliminate circulating glucose in response to the hormone. In the context of the present invention, glucose intolerance can be measured by techniques known to the skilled artisan such as oral glucose tolerance test (OGTT). Glucose intolerance can also be assessed by measuring glucose circulating in the blood, as described in Example 3. In particular, glucose intolerance can be measured by intraperitoneal injection of glucose followed by subsequent measurement of blood glucose, in particular measurements of glucose induced insulin secretion at different time points, for example after 0, 15, 30, 60, 90, 120 and 180 minutes, or for example with an intraperitoneal glucose tolerance test (IPGTT) as defined elsewhere herein (see Example 3). In the context of the present invention, insulin tolerance can be measured by measuring the levels of blood glucose at several time points after intraperitoneal injection of insulin, for example with an intraperitoneal insulin tolerance test (ITT) as defined elsewhere herein (see Examples 3 and 4).

In particular, in the context of the present invention the term “diabetes” includes type 1 and type 2 diabetes (also called juvenile and adult-onset, respectively), gestational diabetes, prediabetes, insulin resistance, and glucose intolerance. In a preferred embodiment, the term “diabetes” refers to prediabetes associated with T2D or T2D.

As such the term “treat”, “treating” or “treatment” as used herein means to reduce (slow down (lessen)), stabilize or inhibit or at least partially alleviate or abrogate the progression of the symptoms associated with the respective disease. Thus, it includes the administration of said fusion protein, preferably in the form of a medicament, to a subject, defined elsewhere herein. Those in need of treatment include those already suffering from the disease, here diabetes. Preferably, a treatment reduces (slows down (lessens)), stabilizes, or inhibits or at least partially alleviates or abrogates progression of a symptom that is associated with the presence and/or progression of a disease or pathological condition. “Treat”, “treating”, or “treatment” refers to a therapeutic treatment. In particular, in the context of the present invention, treating or treatment refers to an improvement of the symptom that is associated with diabetes, as defined elsewhere herein, such as improvement of an impaired glucose tolerance (or glucose intolerance) and/or insulin resistance (or impaired insulin tolerance) in a subject as defined elsewhere herein. By “improved glucose tolerance” (or improvement of an impaired glucose tolerance) is meant that the glucose clearance from blood after a meal or after glucose injection after about 6 hours fasting brings the blood glucose level into a normal range. As the skilled artisan knows, a normal blood glucose level is lower than 140 mg/dL (7.8 mmol/L). A blood glucose level between 140 and 199 mg/dL (7.8 and 11 mmol/L) is considered impaired glucose tolerance, or prediabetes. Improved glucose tolerance can be measured by techniques known to the person skilled in the art, for example, glucose tolerance can be measured by an intraperitoneal glucose tolerance test (IPGTT). By “improved insulin tolerance” is meant that the blood glucose levels measured after administration such as injection of insulin return to normal range as defined herein above; the plasma glucose disappearance rate (KITT) indicates the degree of whole-body insulin sensitivity. Improved insulin tolerance can be measured by techniques known to the person skilled in the art, for example, insulin tolerance can be measured by an intraperitoneal insulin tolerance test (ITT).

The term “prevent”, “preventing”, “prevention” as used herein refers to prophylactic or preventative measures, wherein the subject is to prevent an abnormal, including pathologic, condition in the organism which would then lead to the defined disease, namely diabetes. Thus, it also includes the administration of said fusion protein, preferably in the form of a medicament, to a subject, defined elsewhere herein. Those in need of the prevention include those prone to having the disease, such as diabetes. In other words, those who are of a risk to develop such disease and will thus probably suffer from said disease in the near future.

The term “subject” when used herein includes mammalian and non-mammalian subjects. Preferably the subject of the present invention is a mammal, including human, domestic and farm animals, non-human primates, and any other animal that has mammary tissue. In some embodiment the mammal is a mouse. In a most preferred embodiment the mammal of the present invention is a human. A subject also includes human and veterinary patients. Where the subject is a living human who may receive treatment for a disease or condition as described herein, it is also addressed as a “patient”. In some embodiments, the subject of the present invention is of a risk to develop a disease associated with glucose intolerance and/or insulin resistance, such as prediabetes or T2D. In some embodiments the subject of the present invention suffers from a disease associated with glucose intolerance and/or insulin resistance, such as prediabetes or T2D.

The term “suffering” as used herein means that the subject is not any more a healthy subject. The term “healthy” means that the respective subject has no obvious or noticeable hallmarks or symptoms of the respective disease. This further means that the subject suffering from a disease associated with the presence of glucose intolerance and/or insulin resistance, such as prediabetes or T2D is a subject “in need” of the respective treatment with said fusion protein of the present invention. Those in need of treatment include those already suffering from the disease as well as those prone to having the disease, meaning those in whom the disease is to be prevented (prophylaxis) as mentioned elsewhere herein.

The fusion protein of the present invention or a composition comprising such fusion protein is generally administered to the subject in a therapeutically effective amount. Said therapeutically effective amount is sufficient to inhibit or alleviate the symptoms of disease associated with glucose intolerance, insulin resistance and/or impaired insulin production. By “therapeutic effect” or “therapeutically effective” is meant that the fusion protein of the present invention will elicit the biological or medical response of a tissue, system, animal or human that is being sought by the researcher, veterinarian, medical doctor or other clinician. The term “therapeutically effective” further refers to the inhibition of factors causing or contributing to the disease. The term “therapeutically effective amount” includes that the amount of the fusion protein when administered is sufficient to significantly improve the progression of the disease being treated or to prevent development of said disease. According to a preferred embodiment, the therapeutic effective amount is sufficient to alleviate or heal said disease associated with the glucose intolerance, insulin resistance and/or impaired insulin production.

The term “administering” or “administered” used throughout various aspects of the present invention means that fusion protein or the composition comprising said fusion protein as defined herein are given to the respective subject in an appropriate form and dose and using appropriate measures. The administration of the fusion protein or the composition comprising said fusion protein according to the present invention can be carried out by any method known in the art.

In particular, in the context of the present invention, the fusion protein or the composition comprising the fusion protein as defined elsewhere herein is administered to the subject in a dosage below 3 mg/kg, such as below 2.5 mg/kg, below 2 mg/kg, below 1.5 mg/kg, below 1 mg/kg, or below 0.5 mg/kg. Therefore, the therapeutically effective amount as defined elsewhere herein may be of at least about 2.5 mg/kg and below 3 mg/kg (in other words between about 2.5 mg/kg and 2.9 mg/kg), more preferably of at least about 2 mg/kg and below 3 mg/kg (in other words between about 2 mg/kg and 2.9 mg/kg), more preferably of at least about 1.5 mg/kg and below 3 mg/kg (in other words between about 1.5 mg/kg and 2.9 mg/kg), more preferably of at least about 1 mg/kg and below 3 mg/kg (in other words between about 1 mg/kg and 2.9 mg/kg), more preferably of at least about 0.5 mg/kg and below 3 mg/kg (in other words between about 0.5 mg/kg and 2.9 mg/kg), even more preferably of at least about 0.1 mg/kg and below 3 mg/kg (in other words between about 0.1 mg/kg and 2.9 mg/kg). In a most preferred embodiment, the fusion protein of the invention or the composition comprising said fusion protein is administered in a dosage of about 0.2 mg/kg. In preferred embodiments, the present invention relates to a fusion protein comprising a) a soluble FNDC4 (sFNDC4) or a functional fragment thereof; b) a peptide linker; and c) a Fc-domain as defined elsewhere herein or a composition comprising such fusion protein as defined elsewhere herein for use as a medicament, wherein the fusion protein is administered to the subject as defined elsewhere herein in a dosage below 3 mg/kg according to the present invention.

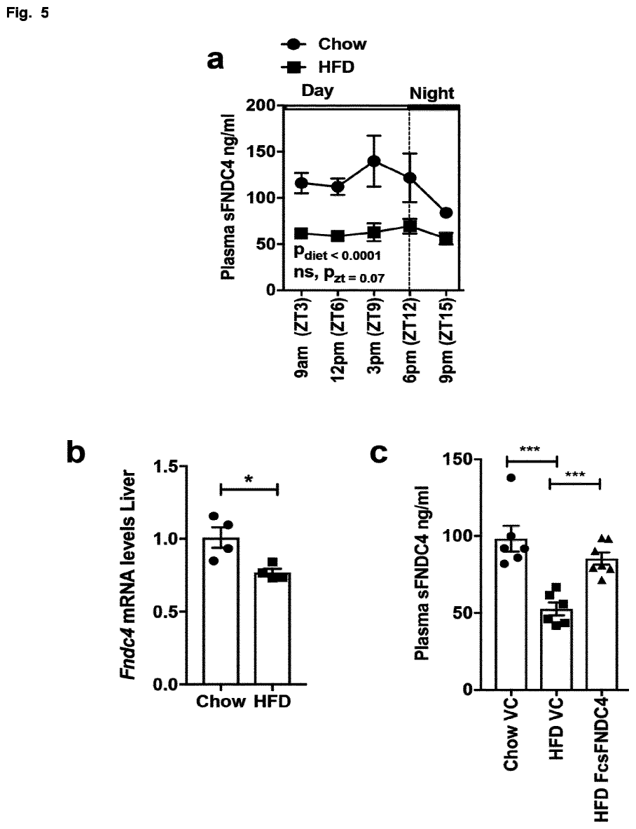

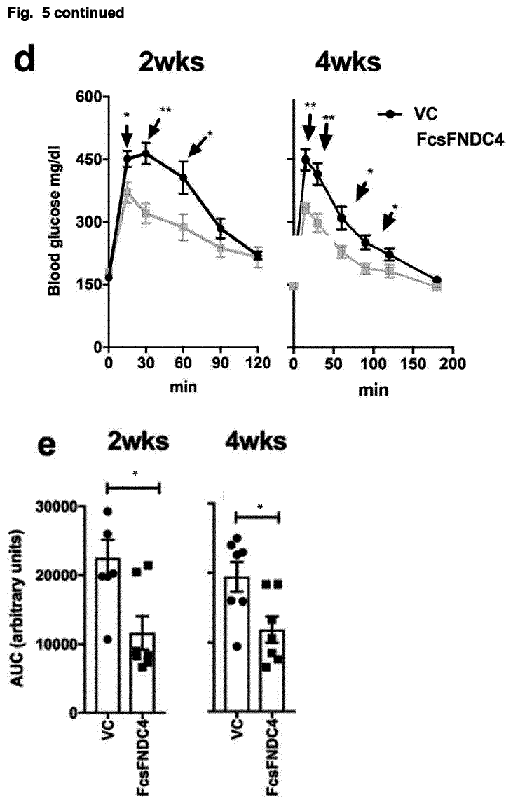

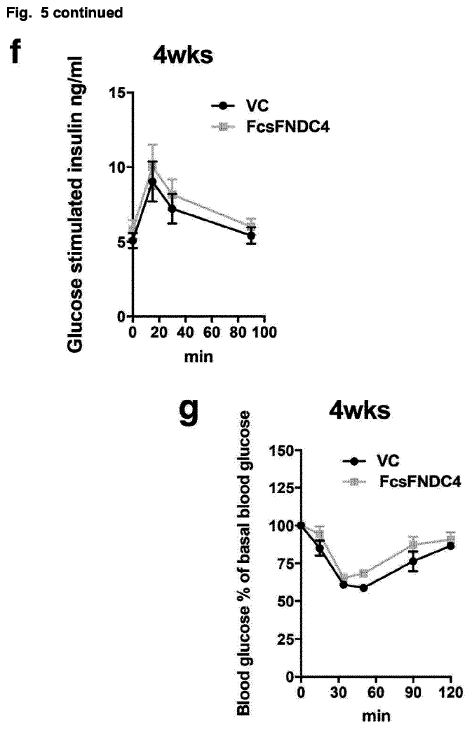

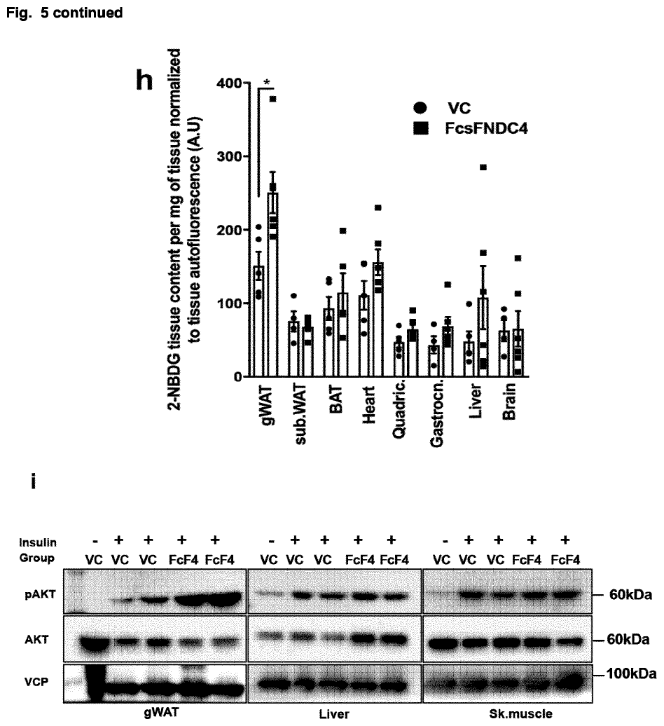

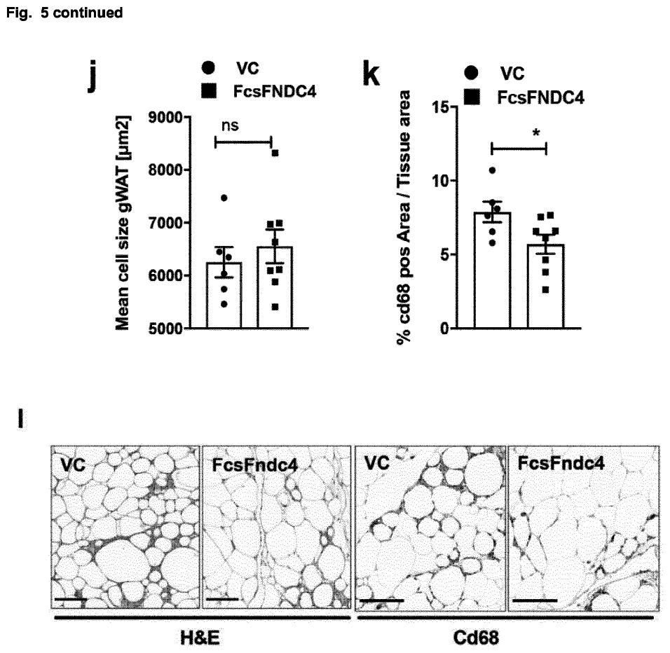

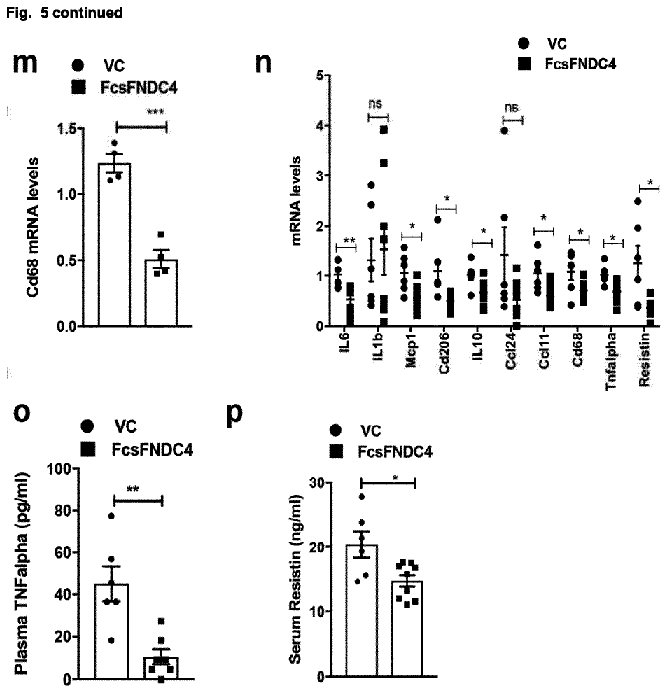

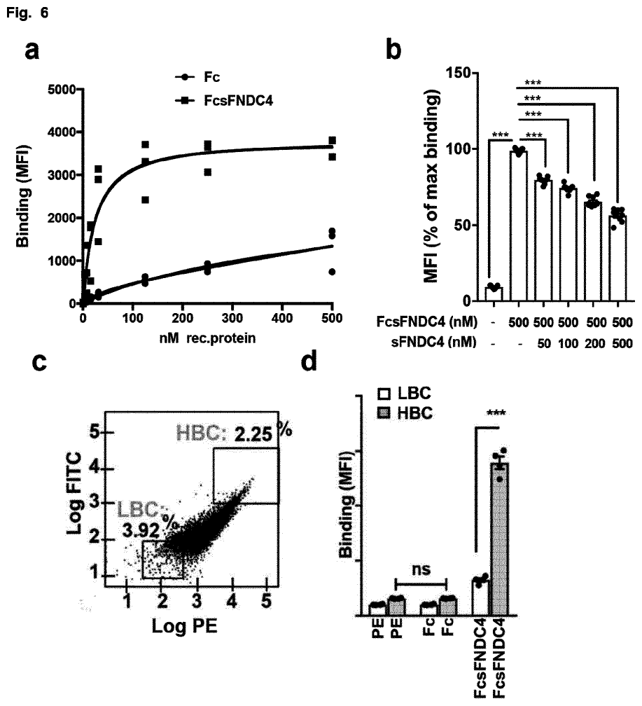

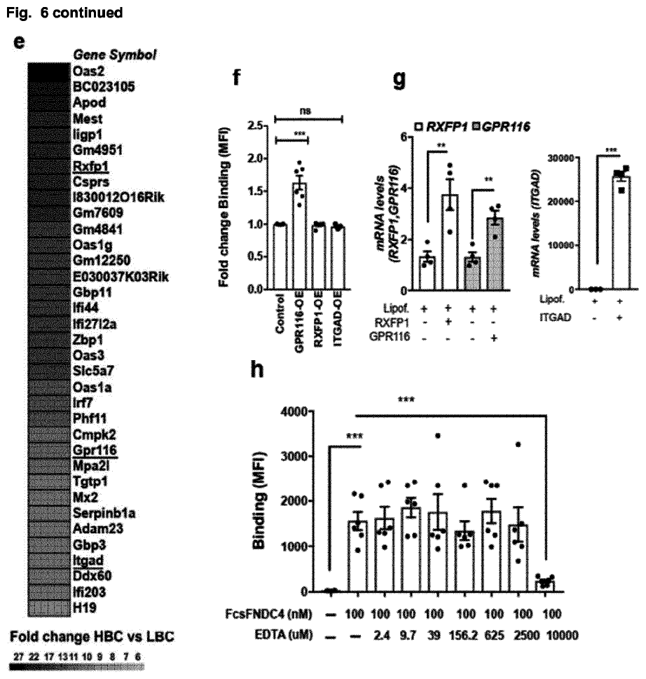

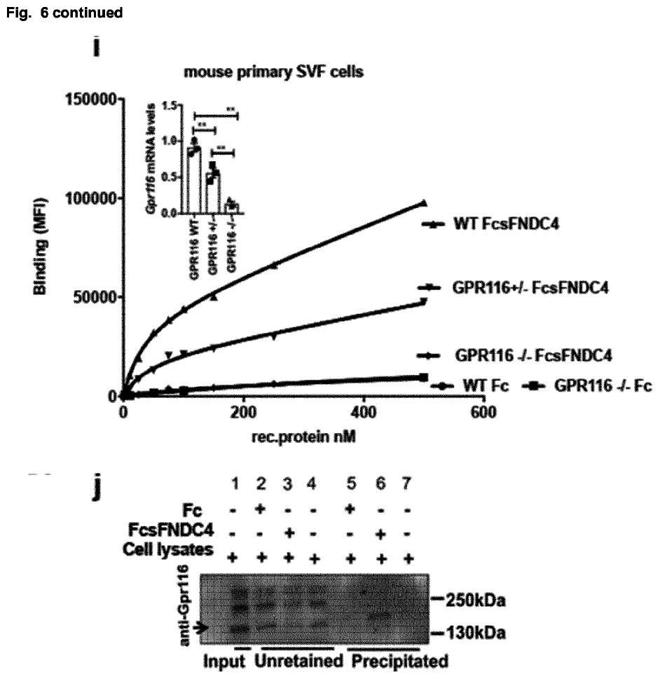

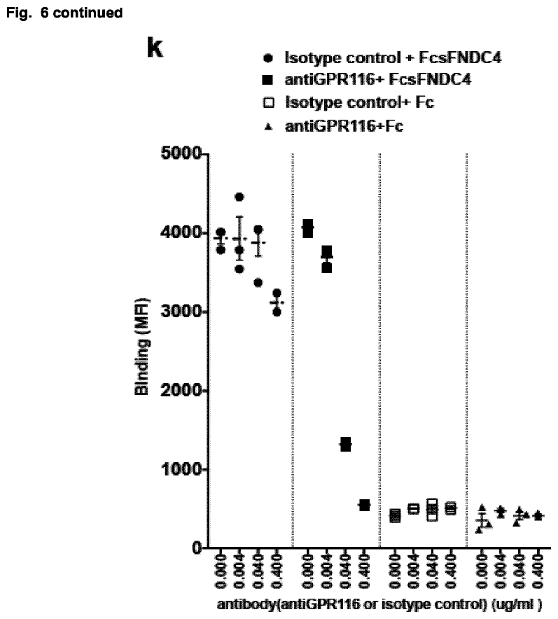

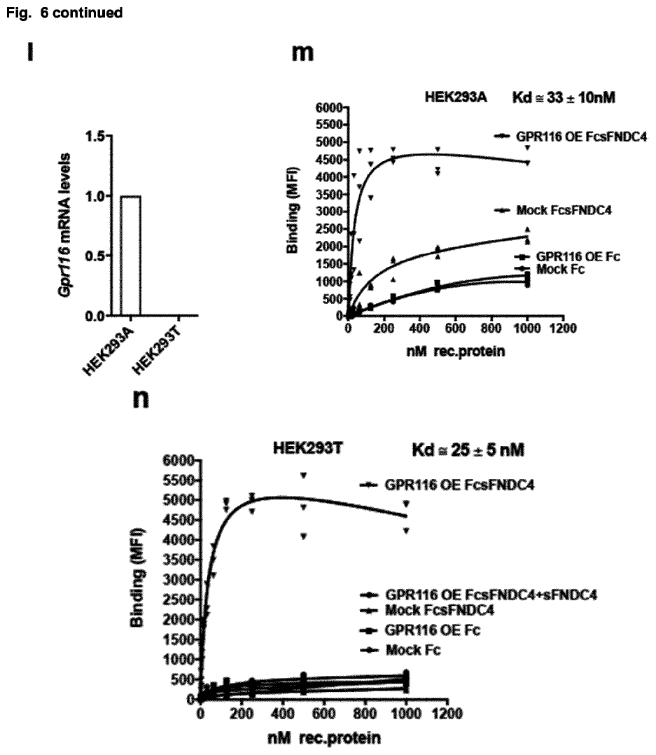

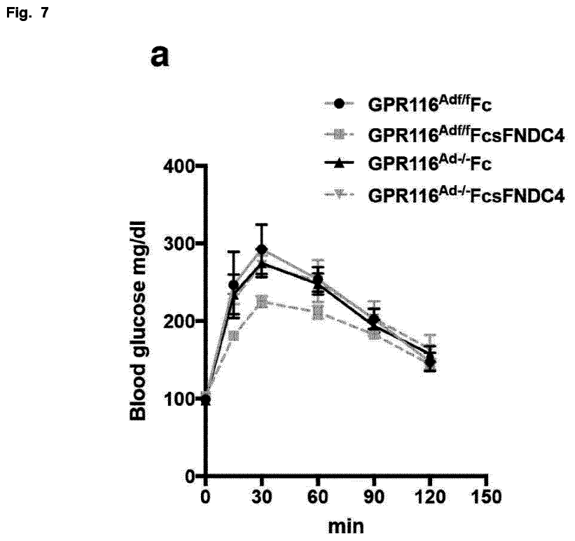

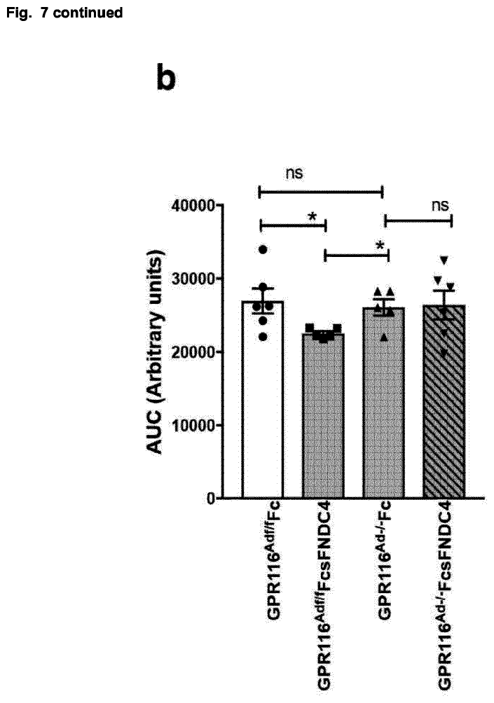

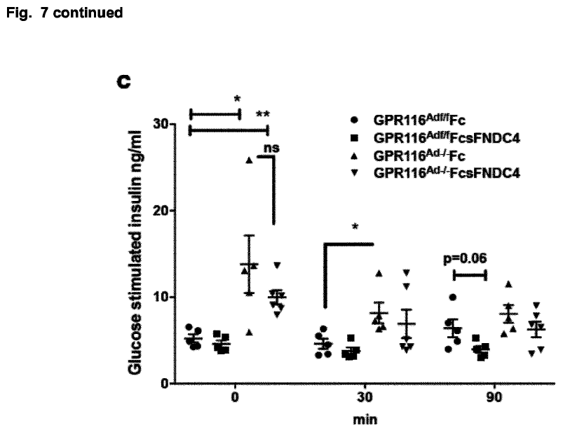

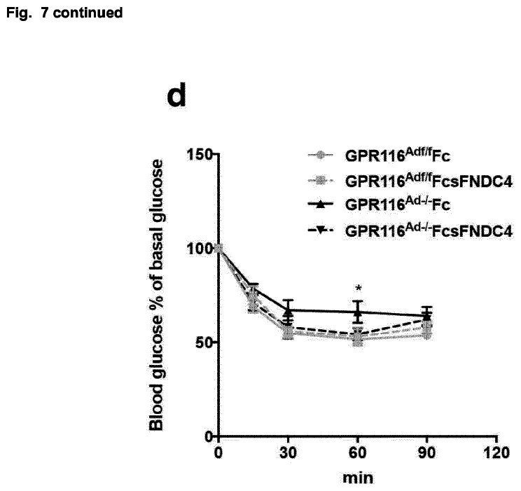

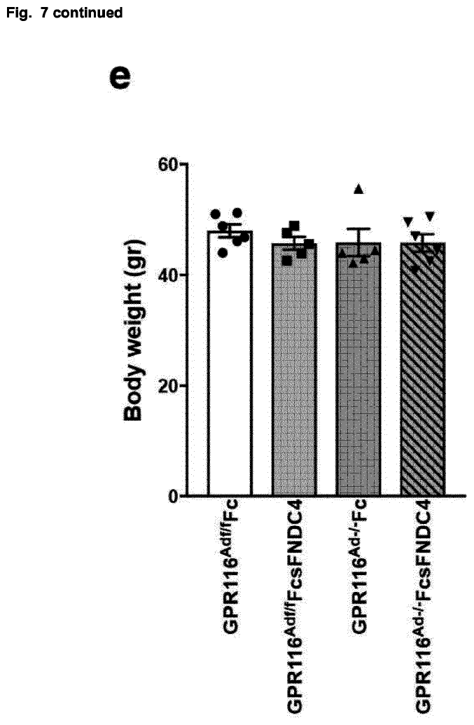

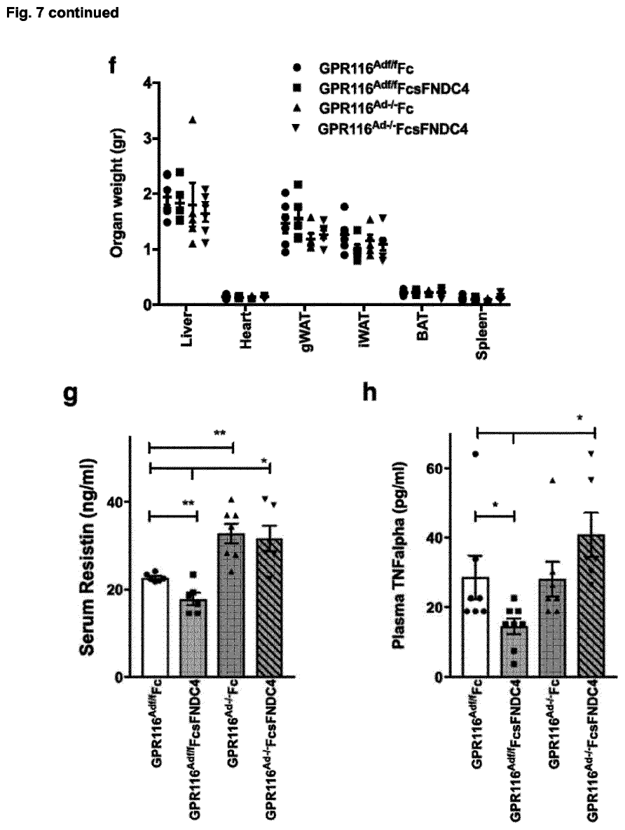

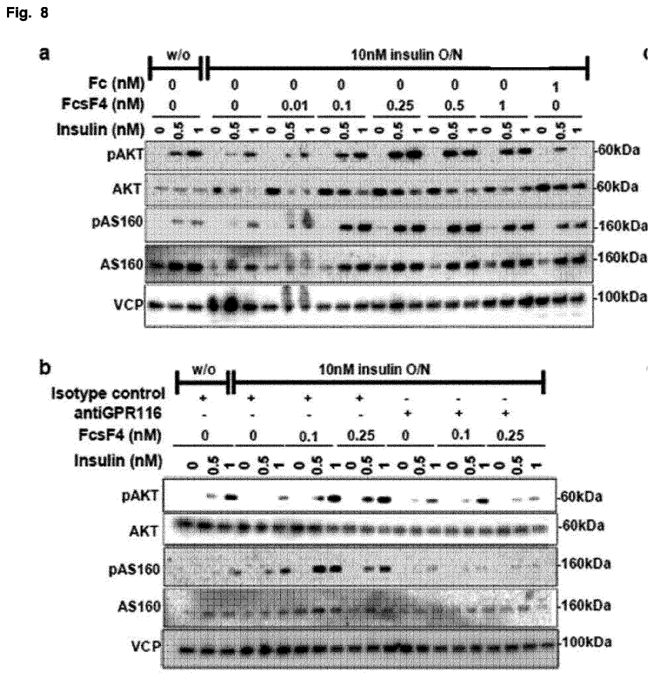

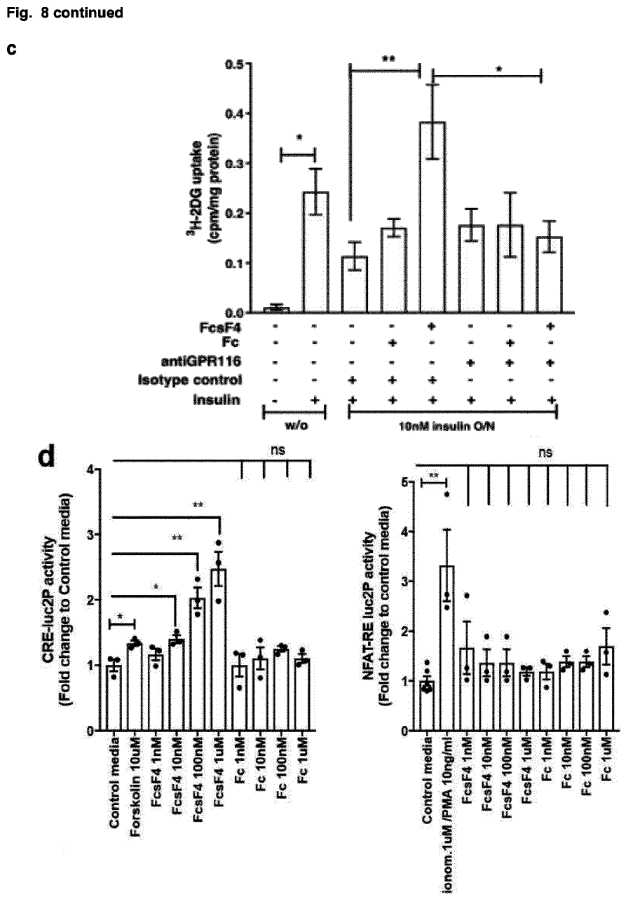

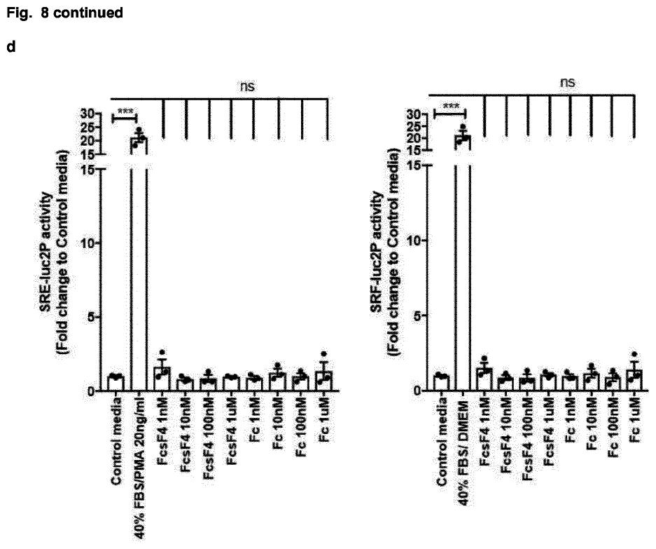

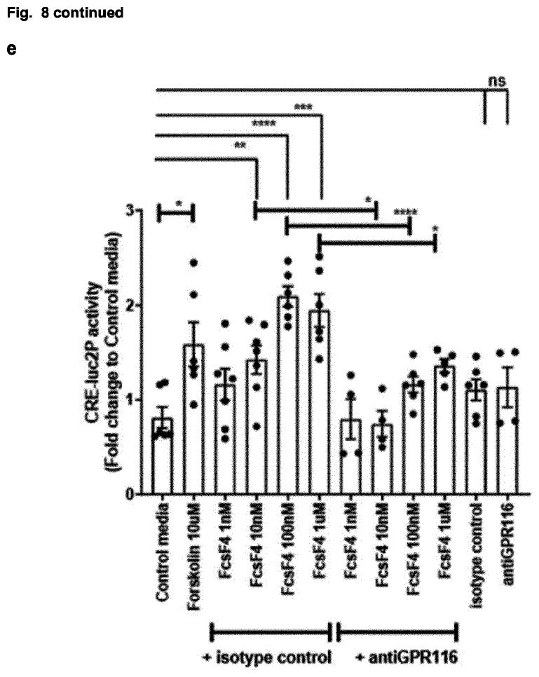

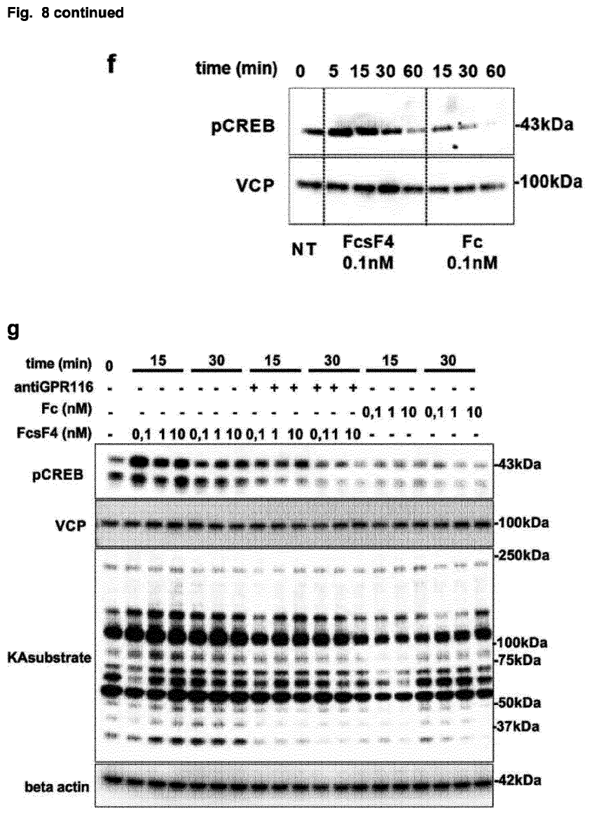

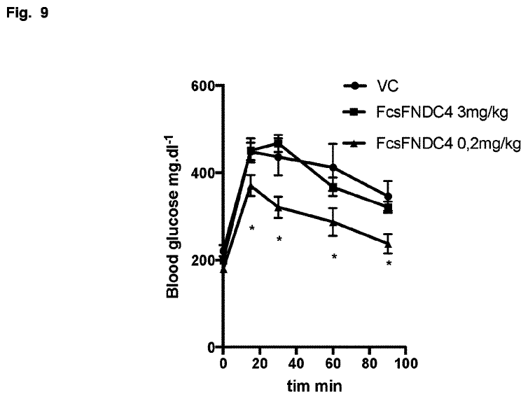

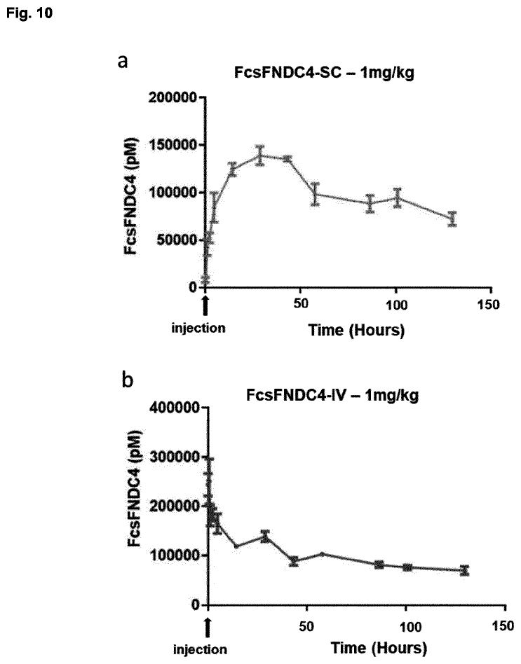

Interestingly, the inventors observed that therapeutic injections of said fusion protein specifically promoted glucose uptake and insulin signaling in the WAT (White Adipose Tissue) upon HFD (High Fat Diet) (FIG. 5). High fat diet refers to a diet consisting of 60% fat. In particular, the inventors surprisingly observed that when the fusion protein as defined elsewhere herein was injected in HFD mice in high dose of 3 mg/kg compared to a low dose of 0.2 mg/kg, the administration of the fusion protein improved glucose tolerance in HFD in a low dose of 0.2 mg/kg (FIG. 9). In sum, the novel fusion protein modified according to the present invention showed sustained metabolic effects at a dose of 0.2 mg/kg, i.p. (intraperitoneal) injection (see FIG. 5).

In this context, the fusion protein or the composition comprising the fusion protein as defined elsewhere herein for use in the treatment/prevention of diabetes, is administered to a subject as defined elsewhere herein, by injection or by infusion, preferably by injection. Even more preferably, the administration of the fusion protein is performed intraperitoneally, intravenously, intraarterially, subcutaneously or intramuscularly, most preferably the administration is performed intraperitoneally. In another preferred embodiment, the administration of the fusion protein is performed subcutaneously. Subcutaneous (SC) delivery (under the skin located above the interscapular space) of drugs is mostly preferred in human therapeutics.

Where the fusion protein or the composition comprising said protein is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. Preferably, where the composition is administered by injection, saline is provided so that the fusion protein is mixed prior to administration.

Thus, in a preferred embodiment, said fusion protein or the composition comprising said fusion protein for use in a method of treating and/or preventing diabetes in a subject is injected intraperitoneally to said subject in a dosage of at least about 0.1 mg/kg and below 3 mg/kg. In an even more preferred embodiment, said fusion protein or the composition comprising said fusion protein for use in a method of treating and/or preventing diabetes in a subject is injected intraperitoneally to said subject in a dosage of about 0.2 mg/kg. In a most preferred embodiment, said fusion protein or the composition comprising said fusion protein for use in a method of treating and/or preventing diabetes in a subject is injected subcutaneously to said subject in a dosage of about 0.2 mg/kg.

In particular, the fusion protein or the composition comprising the fusion protein for the use in the treatment/prevention of diabetes, is administered to the subject as defined elsewhere herein, at least about 8 times. Thus, said administration of the fusion protein or the composition comprising said fusion protein for the use comprises at least about 8 administrations. At least about 8 administrations as defined elsewhere herein may refer to administrations every other day for at least about 2 weeks, such as administering said fusion protein or said composition comprising the fusion protein of the invention at least at day 0, 2, 4, 6, 8, 10, 12, 14. In a preferred embodiment, said administering of said fusion protein or the composition comprising said fusion protein comprises at least about 8 administrations within one month (four weeks). In this context, at least about 8 administrations within one month refers to one month of administrations as defined elsewhere herein every other day comprising at least about 8 administrations as a minimum in said month. Thus, the fusion protein or the composition comprising the fusion protein for the use in the treatment/prevention of diabetes may be administered for a total of 16 administrations every other day within one month, such as administering said fusion protein or said composition comprising the fusion protein of the invention at day 0, 2, 4, 6, 8, 10, 12, 14, 16, 18, 20, 22, 24, 26, 28, 30.

When the fusion protein or the composition comprising the fusion protein is administered subcutaneously, said administration can be performed once a week or according to the half-life of said protein (FIG. 10) every about 8.9 days, preferably within one month (four weeks).

Therefore, the fusion protein or the composition comprising the fusion protein for the use in the treatment/prevention of diabetes of the present invention, is preferably administered in combination with an additional therapeutic agent (drug). Drugs or therapeutic agents useful in this regard include without limitation drug-like molecules, proteins, peptides, and small molecules. Protein therapeutic agents include, without limitation peptides, enzymes, antibodies, structural proteins, receptors and other cellular or circulating proteins as well as fragments and derivatives thereof, preferably an additional therapeutic agent/drug in the context of the present invention may be a drug for the use in diabetes as it is known to a person skilled in the art, especially for combinatorial therapy in diabetes. In some embodiments, the therapeutic agent includes, but is not limited to sodium-glucose cotransporter type 2 (SGLT-2) inhibitor, metformine. Preferably, an additional therapeutic agent/drug administered in combination with the fusion protein of the present invention is metformine.

Additionally, the fusion protein or the composition comprising the fusion protein for the use in the treatment/prevention of diabetes of the present invention improves glucose tolerance defined elsewhere herein in the subject. In particular, the fusion protein or the composition comprising the fusion protein for the use in the treatment/prevention of diabetes of the present invention has binding affinity to the G-protein coupled receptor GPR116, preferably, has binding affinity to the N-terminus of the GPR116 receptor as already described elsewhere herein (FIG. 6).

Surprisingly, the inventors showed that the fusion protein for the use in the treatment of diabetes of the present invention, improves glucose tolerance by specifically binding to the GPR116 receptor as it is defined elsewhere herein. In particular, the inventors observed that the fusion protein interaction with GPR116 was required for improving glucose tolerance (FIG. 7). In particular, the inventors showed that said effect is specific to the GPR116 receptors found in adipose tissue cells. As used herein, the term “adipose tissue cell” refers to cells comprised by the adipose tissue, wherein the adipose tissue is classified, depending on location, in perirenal fat and visceral fat, and depending on structure, in white adipose tissue and brown adipose tissue. In the context of the present invention, the “adipose tissue cells” preferably refer to white adipose tissue (WAT) cells. It is intriguing that despite the wild expression of GPR116 in metabolic tissues, such as muscle and liver, FcsFNDC4 effects are mediated exclusively by adipose tissue GPR116 (FIG. 7 and FIG. 8).

The invention also relates to a fusion protein as defined elsewhere herein for use in a method of preventing and/or treating inflammation in a subject comprising administering to the subject a therapeutically effective amount of said fusion protein. The term “inflammation” may refer to mild obesity and/or T2D induced/related inflammation and/or severe inflammation, preferably to mild obesity and/or T2D induced/related mild inflammation. As mentioned earlier, T2D for example involves mild chronic inflammation, so that T2D related mild inflammation can be prevented and/or treated by using said fusion protein as defined elsewhere herein as anti-inflammatory agent. The paragraphs referring to the treatment of diabetes above may be applicable, where necessary, to the further second medical use of said fusion protein as defined elsewhere herein as anti-inflammatory agent.

### Compositions

The present invention also relates to a composition comprising the fusion protein of the invention. The present invention also relates to a composition comprising at least one fusion protein as defined elsewhere herein. Each definition herein in context of the composition can thus also be applicable when said composition comprises at least one fusion protein. Said composition either refers to a diagnostic or to a pharmaceutical composition. When the fusion protein is for diagnostic purposes (in vitro) as defined elsewhere herein, said composition comprising said fusion protein refers to a diagnostic composition. When the fusion protein is administered to a subject as a therapeutic and preferably be administered in combination with an additional therapeutic agent as defined elsewhere herein for therapeutic purposes, said composition comprising said fusion protein refers to a pharmaceutical composition. Moreover, the present invention relates to the use of a fusion protein as disclosed herein above for the preparation of a diagnostic or pharmaceutical composition.

In accordance with the present invention, the term “pharmaceutical composition” relates to a composition for administration to a patient, preferably a human patient. Pharmaceutical compositions or formulations are usually in such a form as to allow the biological activity of the active ingredient (the fusion protein of the present invention) to be effective and may therefore be administered to a subject for therapeutic use as described herein. In a preferred embodiment, the pharmaceutical composition is a composition for intraperitoneal, intravenous, intraarterial, subcutaneous, intramuscular, parenteral, trans-dermal, intra-luminal, intra-thecal and/or intranasal administration or for direct injection into tissue. It is in particular envisaged that said composition is administered to a patient via infusion or injection, preferably by injection. Administration of the suitable compositions is preferably intravenously, intra-peritoneally, intraarterially, subcutaneously, intra-muscularly. In a preferred embodiment, the administration of the composition is performed intraperitoneally or subcutaneously, even more preferably subcutaneously. The pharmaceutical compositions can be administered to the subject at a suitable dose as defined elsewhere herein. The dosage regimen will be determined by the attending physician and by clinical factors. As is well known in the medical arts, dosages for any one patient depend upon many factors, including the patient's size, body surface area, age, the particular compound to be administered, sex, time and route of administration, general health, and other drugs being administered concurrently.

The term “diagnostic composition” when used herein refers to a composition comprising at least one fusion protein of the present invention, which can be applied for use in diagnosis. Said diagnostic composition is used for stratifying a subject with diabetes by determining the level of circulating soluble FNDC4 in vitro as defined elsewhere herein. Preferably, a sample obtained from a subject as defined herein is contacted with the diagnostic composition comprising the fusion protein as defined elsewhere herein.

The present invention may also encompass the composition as defined herein, further comprising at least one pharmaceutically or diagnostically acceptable carrier (also known as excipient or diluent). Hence, the therapeutic or diagnostic composition of the present invention further comprises a pharmaceutically or diagnostically acceptable carrier, diluent or excipient. Said terms can be used interchangeably. Said pharmaceutically acceptable carrier (also called excipient or diluent) includes any excipient/carrier/diluent that does not itself elicit an adverse reaction harmful to the subject receiving the pharmaceutical composition. Said diagnostically acceptable carrier includes also any carrier that does not itself elicit an adverse reaction, which would be harmful when used in in vitro diagnosis. Suitable excipients are typically large, slowly metabolized macromolecules such as proteins, polysaccharides, polylactic acids, polyglycolic acids, polymeric amino acids, amino acid copolymers and lipid aggregates such as, e.g. oil droplets or liposomes. The carrier used in combination with the fusion protein of the present invention may be water-based and forms an aqueous solution. An oil-based carrier solution containing the compound of the present invention is an alternative to the aqueous carrier solution. Either aqueous or oil-based solutions further contain thickening agents to provide the composition with the viscosity of a liniment, cream, ointment, gel, or the like. Suitable thickening agents are well known to those skilled in the art. Alternative embodiments of the present invention can also use a solid carrier containing the diagnostic compound for use in diagnosis as disclosed elsewhere herein. This enables the alternative embodiment to be applied via a stick applicator, patch, or suppository. The solid carrier further contains thickening agents to provide the composition with the consistency of wax or paraffin.

Pharmaceutically or diagnostically acceptable excipients according to the present invention include, by the way of illustration and not limitation, disintegrants, binding agents, adhesives, wetting agents, polymers, lubricants, gliands, substances added to mask or counteract a disagreeable texture, taste or odor, flavors, dyes, fragrances, and substances added to improve appearance of the composition. Acceptable excipients include lactose, sucrose, starch powder, maize starch or derivatives thereof, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, saline, dextrose, mannitol, lactose, lecithin, albumin, sodium glutamate, cysteine hydrochloride, and the like. Examples of suitable excipients for soft gelatin capsules include vegetable oils, waxes, fats, semisolid and liquid polyols. Suitable excipients for the preparation of solutions and syrups include, without limitation, water, polyols, sucrose, invert sugar and glucose. Suitable excipients for injectable solutions include, without limitation, water, alcohols, polyols, glycerol, BSA and vegetable oils. The diagnostic compositions can additionally include preservatives, solubilizers, stabilizers, wetting agents, emulsifiers, sweeteners, colorants, flavorings, buffers, coating agents, or antioxidants. Suitable pharmaceutical and diagnostic carriers are described in Remington's Pharmaceutical Sciences, Mack Publishing Company, a standard reference text in this field.

Further, the excipients of the pharmaceutical and/or the diagnostic composition may also refer to diluents such as, e.g. water, saline, glycerol, BSA; ethanol, bacteriostatic water for injection (BWFI), Ringer's solution, dextrose solution, or aqueous solutions of salts and/or buffers etc. Furthermore, substances necessary for formulation purposes may be comprised in said compositions as acceptable excipients such as emulsifying agents, stabilizing agent, surfactants and/or pH buffering substances known to a person skilled in the art.

Said stabilizing agent/stabilizer may act as a tonicity modifier. The term “stabilizing agent” refers to an agent that improves or otherwise enhances stability of the formulation. A stabilizing agent which is a tonicity modifier may be a non-reducing sugar, a sugar alcohol or a combination thereof. The tonicity modifiers of the compositions of the present invention ensure that the tonicity, i.e., osmolarity, of the solution is essentially the same as normal physiological fluids and may thus prevent post-administration swelling or rapid absorption of the composition because of differential ion concentrations between the composition and physiological fluids. Preferably, the stabilizing agent/tonicity modifier is one or more of non-reducing sugars, such as sucrose or trehalose or one or more of sugar alcohols, such as mannitol or sorbitol, also combinations of non-reducing sugars and sugar alcohols are preferred.

In compositions of the present invention, the addition of surfactants can be useful to reduce protein degradation during storage. The polysorbates 20 and 80 (Tween 20 and Tween 80) are well established excipients for this purpose. Persons having ordinary skill in the art will understand that the combining of the various components to be included in the formulation can be done in any appropriate order. It is also to be understood by one of ordinary skill in the art that some of these chemicals can be incompatible in certain combinations, and accordingly, are easily substituted with different chemicals that have similar properties but are compatible in the relevant mixture.

The term “buffering agent” as used herein, includes those agents that maintain the pH in a desired range. A buffer is an aqueous solution consisting of a mixture of a weak acid and its conjugate base or a weak base and its conjugated acid. It has the property that the pH of the solution changes very little when a small amount of a strong acid or base is added. Buffer solutions are used as a means of keeping pH at a nearly constant value in a wide variety of chemical applications. In general, a buffer when applied in the formulation of the invention preferably stabilizes the fusion protein of the present invention.

Also, the pharmaceutical composition may comprise one or more adjuvants. The term “adjuvant” is used according to its well-known meaning in connection with pharmaceutical compositions. Specifically, an adjuvant is an immunological agent that modifies, preferably enhances, the effect of such composition while having few, if any, desired immunogenic effects on the immune system when given per se. Suitable adjuvants can be inorganic adjuvants such as, e.g., aluminium salts (e.g., aluminium phosphate, aluminium hydroxide), monophosphoryl lipid A, or organic adjuvants such as squalene or oil-based adjuvants, as well as virosomes.

In a preferred embodiment, the excipients of the pharmaceutical and/or the diagnostic composition is PBS (phosphate buffer), BSA and/or glycerol. BSA may be used to keep stable the soluble fusion protein of the present invention. Glycerol may also be used for stabilization of the fusion protein.

Said composition of the present invention may be a liquid, preferably aqueous, composition.

Further comprised herein is a dried or frozen form of the composition as defined herein. Thus, said composition may be stored directly in liquid form for later use, stored in a frozen state and thawed prior to use, or prepared in dried form, such as a lyophilized, air-dried, or spray-dried form, for later reconstitution into a liquid form or other form prior to use.

Thus, it is envisaged that a composition described herein may be stored by any method known to one of skill in the art. Non-limiting examples include cooling, freezing, lyophilizing, and spray drying the formulation, wherein storage by cooling is preferred.

### Kit

The present invention also relates to a kit comprising the fusion protein as defined elsewhere herein or the composition defined elsewhere herein. Thus, when a kit comprises the fusion protein per se, said fusion protein may be provided in a vial or a container. Further, it may be associated with a notice in the form prescribed by a governmental agency regulating the manufacture, use or sale of pharmaceuticals or biological products, reflecting approval by the agency of the manufacture, use or sale of the product for human administration or diagnostics. Said kit may comprise the fusion protein, preferably in a vial or container, in dried form, such as a lyophilized, air-dried, or spray-dried form (in form of a powder), for later reconstitution into a liquid form or other form prior to use. Further, said kit may also comprise the fusion protein, preferably in a vial or container, in a frozen state, being thawed prior to use. According to the present invention, the kit comprising the fusion protein may further comprise a pharmaceutically or diagnostically acceptable excipient, and/or an adjuvant as defined elsewhere herein. In some embodiments, said excipient and/or said adjuvant as defined elsewhere herein may also be comprised in one or more containers or vials in said kit, meaning said kit additionally comprising either one vial or container comprising said excipient and/or said adjuvant as a mixture or said kit additionally comprising for each component such as the excipient and/or the adjuvant separate vials or containers.

When a kit comprises the composition as defined elsewhere herein, said composition may be a pharmaceutical or a diagnostic composition as defined herein. Said kit comprising the pharmaceutical composition as defined herein may be suitable for administering the pharmaceutical composition to a subject as defined elsewhere herein for therapeutic purposes. Said kit comprising the diagnostic composition as defined herein is preferably suitable for stratifying a subject with diabetes as defined elsewhere herein.

According to the present invention, the compositions as defined herein are preferably provided in one or more containers or vials in said kit (pharmaceutical/diagnostic pack), which may also be associated with a notice in the form prescribed by a governmental agency regulating the manufacture, use or sale of pharmaceuticals or biological products, reflecting approval by the agency of the manufacture, use or sale of the product for human administration or diagnostics.

Thus, the present invention may comprise a kit comprising one vial or container comprising the composition as defined herein comprising the fusion protein, wherein the composition additionally comprises the acceptable excipient and/or the adjuvant as defined herein. Also comprised by the present invention, is a kit comprising one or more vials or containers each comprising the composition as defined herein comprising the fusion protein of the present invention, wherein each composition in said vial or container additionally comprises the acceptable excipient and/or the adjuvant as defined herein.

### In Vitro and In Vivo Applications

The present invention also relates to a method of producing said fusion protein of the present invention. In this context, the fusion protein is produced starting from the nucleic acid coding for the fusion protein by means of genetic engineering methods. In some embodiments, the method can be carried out in vivo, the protein can, for example, be produced in a bacterial or eukaryotic host organism and then isolated/recovered from this host organism or its culture by means known to the person skilled in the art. It is also possible to produce said fusion protein of the present invention in vitro, for example by use of an in vitro translation system. In this context, the in vitro translation may also refer to cell free-protein synthesis as known to the skilled person in the art. Cell-free protein synthesis, also known as in vitro protein synthesis or CFPS, is the production of protein using biological machinery in a cell-free system, that is, without the use of living cells.

When producing the fusion protein in vivo as can be seen in the Example section, a nucleic acid molecule comprising a nucleotide sequence encoding such fusion protein is introduced into a suitable bacterial or eukaryotic host organism (preferably CHO cells) by means of recombinant DNA technology. For this purpose, the host cell is first transformed with a cloning vector that includes a nucleic acid molecule encoding a fusion protein as described herein using established standard methods. The host cell is then cultured under conditions, which allow expression of the heterologous DNA and thus the synthesis of the corresponding fusion protein. Subsequently, the fusion protein is isolated/recovered either from the host cell or from the cultivation medium.

The present invention also relates to a method of stratifying a subject with diabetes, comprising a) determining the level of endogenous sFNDC4 or a functional fragment thereof as it is defined elsewhere herein in a test sample obtained from said subject, which has been contacted with the fusion protein or the composition comprising said fusion protein as defined elsewhere herein and then stratifying said subject as suffering from diabetes, if the level of sFNDC4 is decreased relative to a corresponding level of sFNDC4 in a control sample obtained from a healthy subject. In this context, the term “decreased” can mean that the amount of sFNDC4 is decreased by at least about 25%, more preferably by at least about 20%, more preferably by at least about 15%, more preferably by at least about 10% compared to the corresponding levels of sFNDC4 in a control sample obtained from a healthy subject. In preferred embodiments, the amount of sFNDC4 is decreased by at least about 10% compared to the corresponding levels of sFNDC4 in a control sample obtained from a healthy subject.

The term “stratifying” as used herein refers to assigning a likelihood or assessing the risk that a subject may suffer from diabetes as defined elsewhere herein. In other words, it may include susceptibility to the disease (that a subject will suffer from diabetes in the near future). In this context, such method also comprises after having stratified a subject with diabetes, diagnosing said subject with diabetes. Thus, the present invention also comprises a method of stratifying and/or diagnosing a subject with diabetes as defined elsewhere herein. Preferably, said diagnosis is used for stratifying a subject with diabetes by determining the level of circulating soluble FNDC4 in vitro.

The term “diagnosing” or “diagnosis” when used herein means determining or detecting if a subject suffers from a disease. Preferably, said diagnosis is diagnosis of diabetes in a subject. However, where reference is made to “diagnosis” of such a disease, this should be taken to include diagnosis of the disease itself (as a confirmation). Accordingly, the methods of diagnosis disclosed herein may also be employed as methods of providing indications useful in the diagnosis of such a disease.

In accordance with the present invention, the terms “determining”, “measuring”, “evaluating”, “assessing” and “assaying” are used interchangeably and include determining if an element is present or absent. Any suitable form of analysis can be employed in this regard. These terms further include quantitative determinations. Assessing may be relative or absolute. “Determining the level of” includes determining the amount of something present, as well as determining whether it is present or absent.

According to the method of the present invention, a test sample obtained from a subject, includes, but is not limited to, blood, plasma or serum cells. Said obtained sample is then contacted with the fusion protein or the composition comprising said fusion protein as defined elsewhere herein. Thus, a test sample is a sample after being obtained from a subject, which is always then contacted with the fusion protein or the composition of the invention. A control sample is a sample after being obtained from a healthy subject, which is also then contacted with the fusion protein or the composition of the invention. The term “contacting” as used in this context means that the sample is brought together with the fusion protein or the composition as defined herein. Preferably, said fusion protein comprises a fluorophore, an enzyme for producing bioluminescence or an antibody, if determination is done via imaging methods known to a person skilled in the art. Thus, in this context the fusion protein of the invention may be labeled. The label may be selected from the group consisting of a fluorophore, an enzyme for producing bioluminescence and/or an antibody. When said fusion protein of the present invention is a fluorophore (also called fluorochrome or chromophore) it may be any one of a fluorescent dye such as but not limited to Fluorescein (FITC), Alexa Fluor 350, 405, 488, 532, 546, 555, 568, 594, 647, 680, 700, 750, Pacific Blue, Coumarin, Pacific Green, Cy3, Texas Red, PE, PerCP-Cy5, PE-Cy7, Pacific Orange, or a fluorescent protein label such as R-PE or APC, or an expressed fluorescent protein such as CFP, EGFP, GFP or RFP. A suitable enzyme refers to but is not limited to luciferase. The attachment of the label may be either direct or indirect via a linker, if the label may be a fluorophore and/or an enzyme for producing bioluminescence. Said attachment of the label to said fusion protein may also be covalently as defined elsewhere herein.

The terms “contacting”, “bringing into contact”, or “bringing together” as used herein, are not particularly limited and include all means of contacting cells/tissues/samples with the fusion protein of the present invention. For example, for in vitro applications, the fusion protein or the composition of the invention can be added to suspensions or samples in which cultured cells/tissue are kept.

After having contacted the test sample as described herein, for stratifying a subject the level of endogenous sFNDC4 needs to be determined in said test sample. Determination may refer to the fact that said fusion protein may therefore bind to endogenous GRP116 receptor, in this way competing with endogenous sFNDC4. By measuring the amount of fusion protein bound to the GPR116 purified domains, preferably immobilized on an ELISA plate, or alternatively by measuring the amount of fusion protein bound to the GPR116 domains expressed on the surface of cultured cells, the total amount/level of endogenous sFNDC4 in said test and said control sample can be derived/determined, which then may lead to the stratification/diagnosis of said subject with diabetes.

If the level of endogenous sFNDC4 or a functional fragment thereof is decreased relative to a corresponding level of sFNDC4 in a control sample obtained from a healthy subject which also has been contacted with the fusion protein, the subject can be stratified as suffering from diabetes. Thus, if the level of sFNDC4 or a functional fragment thereof is decreased relative to a corresponding level of sFNDC4 in a control sample obtained from a healthy subject, it is indicative that said subject suffers from diabetes. In this context, the term “amount” or “value” can be used interchangeably with ther term “level”. The term “relative to” means “in comparison to” or “compared to”, when used herein.

Preferably, the level of sFNDC4 or a functional fragment thereof is decreased by at least about 10% relative to the corresponding level of sFNDC4 or a functional fragment in said control sample as defined elsehwhere herein. Typically, the disease as described herein is associated with the presence of elevated or increased levels of blood glucose as defined elsewhere herein. Thus, said disease is associated with an increased level of blood glucose in said subject when compared to a subject not suffering from said disease. Thus, the presence of said increased level of blood glucose, is above the normal level of blood glucose in a particular tissue in said subject as defined elsewhere herein. These values can be inferred by the measured levels of endogenous sFNDC4 which, as defined elsewhere herein, correlate with the levels of blood glucose. After having stratified said subject with diabetes according to the method as defined herein, the fusion protein or the composition comprising said fusion protein as defined elsewhere herein may then be used to prevent or treat diabetes in said subject in need thereof as it has also been described herein.

TABLE 1
Summary of the sequence.

| SEQ ID NO. | What | Sequence |
|---|---|---|
| 1 | Soluble Fibronectin | DRPPSPVNVTVTHLRANSATVSWDVPEGNIVIGYSISQQRQ |
| | type III domain- | NGPGQRVIREVNTTTRACALWGLAEDSDYTVQVRSIGLRGE |
| | containing protein 4 | SPPGPRVHFRTLKGSDRLPSNSSSPGDITVEGLDGERPLQT |
| | Homo sapiens | GE |
| 2 | Soluble Fibronectin | DRPPSPVNVTVTHLRANSATVSWDVPEGNIVIGY |
| | type III domain- | SISQQRQNGPGQRVIREVNTTTRACALWGLAEDS |
| | containing protein 4 | DYTVQVRSIGLRGESPPGPRVHFRTLKGSDRLPS |
| | Mus musculus | NSSSPGDITVEGLDGERPLQTGE |
| 3 | Peptide Linker | ENLTFQGX₈X₉ |
| | Artificial sequence | |
| | X₈ = any natural | |
| | occurring amino acid | |
| | X₉ = any natural | |
| | occurring amino acid | |
| 4 | FcsFNDC4 | HHHHHHADLIEGRGDPKSCDKPHTCPLCPAPELLG |
| | Comprising human | GPSVFLFPPKPKDTLMISRTPEVTCVVVDVSHEDPE |
| | soluble FNDC4 | VKFNWYVDGVEVHNAKTKPREEQYNSTYRVVSVLT |
| | | VLHQDWLNGKEYKCKVSNKALPAPIEKTISKAKGQP |
| | | REPQVYTLPPSRDELTKNQVSLTCLVKGFYPSDIAVE |
| | | WESNGQPENNYKATPPVLDSDGSFFLYSKLTVDKSR |
| | | WQQGNVFSCSVMHEALHNHYTQKSLSLSPGKAAEN |
| | | LTFQGAARPPSPVNVTVTHLRANSATVSWDVPEGNIV |
| | | IGYSISQQRQNGPGQRVIREVNTTTRACALWGLAEDS |
| | | DYTVQVRSIGLRGESPPGPRVHFRTLKGSDRLPSNSS |
| | | SPGDITVEGLDGERPLQTGE |
| 5 | FcsFNDC4 | HHHHHHAKTTPPSVYPLAPGSAAQTNSMVTLGCLVK |
| | Comprising mouse | GYFPEPVTVTWNSGSLSSGVHTFPAVLQSDLYTLSS |
| | soluble FNDC4 | SVTVPSSPRPSETVTCNVAHPASSTKVDKKIVPRDCG |
| | | CKPCICTVPEVSSVFIFPPKPKDVLTITLTPKVTCVVVD |
| | | ISKDDPEVQFSWFVDDVEVHTAQTQPREEQFNSTFR |
| | | SVSELPIMHQDWLNGKEFKCRVNSAAFPAPIEKTISK |
| | | TKGRPKAPQVYTIPPPKEQMAKDKVSLTCMITDFFPE |
| | | DITVEWQWNGQPAENYKNTQPIMNTNGSYFVYSKLN |
| | | VQKSNWEAGNTFTCSVLHEGLHNHHTEKSLSHSPGK |
| | | RPPSPVNVTVTHLRANSATVSWDVPEGNIVIGYSISQQ |
| | | RQNGPGQRVIREVNTTTRACALWGLAEDSDYTVQVR |
| | | SIGLRGESPPGPRVHFRTLKGSDRLPSNSSSPGDITVE |
| | | GLDGERPLQTGE |
| 6 | Human IgG1 | ADLIEGRGDPKSCDKPHTCPLCPAPELLGGPSVFLFPP |
| | sequence | KPKDTLMISRTPEVTCVVVDVSHEDPEVKFNWYVDGV |
| | | EVHNAKTKPREEQYNSTYRVVSVLTVLHQDWLNGKEY |
| | | KCKVSNKALPAPIEKTISKAKGQPREPQVYTLPPSRDEL |
| | | TKNQVSLTCLVKGFYPSDIAVEWESNGQPENNYKATPP |
| | | VLDSDGSFFLYSKLTVDKSRWQQGNVFSCSVMHEALH |
| | | NHYTQKSLSLSPGKAA |
| 7 | Mouse IgG1 | AKTTPPSVYPLAPGSAAQTNSMVTLGCLVKGYFPEPVT |
| | sequence | VTWNSGSLSSGVHTFPAVLQSDLYTLSSSVTVPSSPRP |
| | | SETVTCNVAHPASSTKVDKKIVPRDCGCKPCICTVPEVS |
| | | SVFIFPPKPKDVLTITLTPKVTCVVVDISKDDPEVQFSWF |
| | | VDDVEVHTAQTQPREEQFNSTFRSVSELPIMHQDWLN |
| | | GKEFKCRVNSAAFPAPIEKTISKTKGRPKAPQVYTIPPP |
| | | KEQMAKDKVSLTCMITDFFPEDITVEWQWNGQPAENY |
| | | KNTQPIMNTNGSYFVYSKLNVQKSNWEAGNTFTCSVL |
| | | HEGLHNHHTEKSLSHSPGK |
| 8 | TEV protease site | ENLX₄FQX₇ |
| | X₄ = Tyr or Thr | |
| | X₇ = Gly or Ser | |

| | | |
|---|---|---|
| 9 | Signal peptide in | MPPGPCAWPPRAALRLWLGCVCFALVQAD |
| | expression vector | |

### EXAMPLES OF THE INVENTION

The following examples illustrate the invention. These examples should not be construed as to limit the scope of this invention. The examples are included for purposes of illustration and the present invention is limited only by the claims.

### Material and Methods

Animals: All mice were housed in a temperature-controlled (20-22° C.) room on a 12-h light/dark cycle. Mice were fed a chow diet or HFD research diets (45% fat and 60% fat) where indicated. Chow fed mice were housed 4-5 mice per cage and mice on HFD were housed 3-4 mice per cage. Experiments were performed in age and sex matched mice.

Littermates of the same sex were randomly assigned to experimental groups. GPR116flox/flox (Yang, M. Y., Hilton, M. B., Seaman, S., Haines, D. C., Nagashima, K., Burks, C. M., Tessarollo, L., Ivanova, P. T., Brown, H. A., Umstead, T. M., et al. (2013). Essential Regulation of Lung Surfactant Homeostasis by the Orphan G-protein Coupled Receptor GPR116. Cell Rep. 3, 1457-1464) and Adiponectin-Cre transgenic mice were crossed to produce the adipose specific GPR116 conditional knockout mice: GPR116^{Ad-/-}, which is Adiponectin Cre positive and GPR116 flox/flox mice. Adiponectin Cre negative GPR116^{Adf/f} mice were used as controls.

Wild type mice for rec. protein injections, high fat diets and primary cells isolation were C57BL6N, male mice and they were purchased from Charles River Laboratory.

All experiments were conducted in accordance with European Directive 2010/63/EU on the protection of animals used for scientific purposes and were performed with permission from the Animal Care and Use Committee (N169/13, N412/12, N187/12 860 and 49-2017, 15-164).

Cell lines: Human embryonic kidney 293 (HEK293) cells were established from female fetus. NIH3T3 fibroblasts are of mouse fibroblasts, which lack differentiation capacity to mature adipocytes. 3T3L1 are white adipose tissue mouse fibroblasts, with differentiation capacity. HepG2 is a human liver cancer cell line. All cell lines were cultured in DMEM high glucose media with 10% FBS and 1% Penicillin-868 Streptomycin (P/S) at 37° C. in 5% CO2. Immortalized SVF cells used for FACS based receptor screening were derived from male 129SVE mice, as described in (Duteil, D. et al. Lsd1 prevents age-programed loss of beige adipocytes. Proc Natl Acad Sci U 1198 S A 114, 5265-5270 2017) and (Wu, J., Boström, P., Sparks, L. M., Ye, L., Choi, J. H., Giang, A.-H., Khandekar, M., 826 Virtanen, K. A., Nuutila, P., Schaart, G., et al. (2012). Beige Adipocytes Are a Distinct Type of Thermogenic Fat Cell in Mouse and Human. Cell 150, 366-376).

Primary cell cultures: Primary islets were isolated from the pancreas of 8-13 week old C57BL/6N mice via collagenase P (Roche) digestion as described before (Szot, G. L., Koudria, P., and Bluestone, J. A. (2007). Murine pancreatic islet isolation. Journal of visualized experiments: JoVE, 255-255) followed by a centrifugation step using an Optiprep density gradient (Sigma). Isolated islets were handpicked twice and incubated overnight in RPMI supplemented with 10% v/v FBS and 1% v/v PS for recovery.

Expression and purification of recombinant proteins: FcsFNDC4 generation: 6×His Fc sFNDC4 (FcsFNDC4) fusion protein and 6×His Fc (Fc) control were expressed using a pEFIRES expression vector. DNA fragment coding signal peptide (SP) from Fndc5 fused to 6×His Fc was synthesized by GeneScript USA Inc. and cloned into pEFIRES modified multiple cloning site, using NheI and NotI restriction sites. The extracellular part of FNDC4 (sFNDC4) was PCR amplified using mouse clone MR223815 (OriGene) as a template with the set of primers: forward GAGAGCGGCCGCTCGACCTCCCTCTCCTGTG (SEQ ID NO: 10), reverse GAGAGAATTCATTCCCCTGTCTGCAATGGC (SEQ ID NO: 11). The amplified sFNDC4 fragment was cloned into a SP 6×His Fc pEFIRES vector using NotI and EcoRI restriction sites to produce SP 6×His hFc sFNDC4 pEFIRES expression constructs respectively. These constructs were transfected to CHOS cells and stable cell lines were selected using puromycin as a selection agent. For protein production stable CHOS suspension cultures were grown in OptiCHO medium (Life Technologies) supplemented with Ala Glu (Sigma). Culture supernatants were loaded onto HisTrap Excel columns (GE Healthcare) in 5 mM imidazole containing Column loading buffer (1M NaCL) with protease inhibitors, pH 7.4, washed with 5 mM and 20 mM imidazole containing wash buffers (0.2 M NaCL, 0, NaHPO4, pH 7.4) and eluted with 250 mM imidazole elution buffer (0.2 M NaCL, 0.02 M NaHPO4, pH 7.4). Eluted protein was dialyzed against PBS buffer. Newly synthesized proteins were concentrated using 3 kDa Amicon Ultra 15 centrifugal units (Merck Millipore) to 1 mg/ml and snap frozen prior to the analysis. 0.5 ml of the protein sample was loaded onto Superdex 10/300 GL (GE Healthcare) equilibrated in PBS buffer. Superdex 10/300 GL was calibrated using High Molecular Weight (HMW) Kit (GE Healthcare).

Non Fc fused FNDC4: FNDC4 (aa40 160, UniProtKB Q3TR08 Mouse FNDC4) was cloned into a pETM11 vector for bacterial expression. After expression of 1 L in TB medium, induction with IPTG and overnight growth at 20° C., cells were collected and frozen at 80° C. until further usage. Cells were lysed in 20 mM tris pH 8.5, 150 mM NaCl, 10 mM imidazole, 5% glycerol, 2 mM @mercaptoethanol and supplemented with protease inhibitor. Prior to sonication and lysate clearance by 45 min centrifugation, at 25000 rpm, supernatant was applied to a prepacked nickel column. After washing with 20 times column volume, protein was eluted in an imidazole gradient to a final concentration of 350 mM imidazole. The His-tag version of FNDC4 was then further purified over a size exclusion chromatography column S200, whereas untagged FNDC4 was first supplemented with TEV protease, to cleave the His-tag when simultaneously dialyzed overnight against a buffer with 10 mM imidazole. Prior to gel filtration, cleaved FNDC4 domain was subjected to a nickel column to remove non-cleaved His-tag FNDC4 and His-tagged TEV protease. In both cases, the final buffer used was 10 mM Hepes, 100 mM NaCl, 5% glycerol and 1 mM β-mercaptoethanol. Untagged FNDC4 used for the FACS binding competition assays was in PBS buffer.

Transient overexpression of human RXFP1, ITGAD and human GPR116: Open reading frames for human RXFP1 (RC511338), ITGAD (RC224758) and human GPR116 (RC209170) were purchased from OriGene and subcloned into pENTR-CMV vector (Gateway Invitrogen). The plasmids were transfected with Lipofectamin into HEK293 cells with the standard protocol. These cells were used for experiments 48 h post transfection.

FACS binding assay and sorting: The cells were detached by 1 min incubation in prewarmed 0.05% trypsin EDTA and additionally scraped in ice cold PBS. Cells were washed three times in suspended in FACS buffer (PBS with 3% FBS), by in between pelleting using centrifugation in 1000×g for 5 min at 4° C. All steps were performed in cold. Fc block (1:200) was added for 20 min in FACS buffer. Recombinant proteins were then added and incubated with cells at 4° C. for 40 min, washed three times with cold FACS buffer, followed by 40 min incubation (4° C.) with anti-human IgG secondary antibody conjugated with PE (Invitrogen, H10104, 1:200). After the incubation with secondary antibody, cells were washed twice with FACS buffer and then analyzed by analytical FACS. Quantification was performed using the mean phycoerythrin (PE) value within the total cell population (10000 events were recorded). Measurements of fluorescein isothiocyanate (FITC) values were acquired to correct for cell auto fluorescence. For sorting, the cells were labeled as described above and sorted for high or low mean PE and normalized to mean FITC (background fluorescence). The incubation with recombinant proteins was performed in 96-well plates with round bottom. 100000-300000 cells were used. During incubation cells were mixed twice by gentle vortexing. During washes a table centrifuge for plates was used at 600×g for 2 min at 4° C.

FACS binding in the presence of EDTA: The cells were detached by 1 min incubation in prewarmed 0.05% trypsin EDTA and additionally scraped in ice cold PBS. Cells were washed three times Krebs-Ringer buffer with the following composition: 100 mM NaCl, 5 mM KCl, 0.1 mM MgSO4, 0.1 mM CaCl2 0.4 mM K2HPO4, 10 mM HEPES. All steps were performed in cold. Fc block (1:200) was added for 20 min in FACS buffer and suspended. FcsFNDC4 was added at 100 nM final concentration and increasing amount of EDTA (0 mM-10 mM). The rest of the binding protocol was performed as described above, see ‘FACS binding assay and sorting’.

FcsFNDC4 after blocking with anti-GPR116 antibody: HEK239T cells stably overexpressing GPR116 were detached in ice cold PBS by scraping and pelleted by centrifugation 1000×g 5 min at 4° C. and resuspended in FACS buffer (PBS with 3% FBS). Cells were incubated 20 min with Fcblock on ice, followed by 30 min incubation with antiGPR116 or IgG isotype control. At different concentrations. Antibody was removed by centrifugation at 600×g for 2 min and then 100 nM of FcsFNDC4 or Fc rec. protein were added on the cells in FACS buffer for 40 min on ice. Afterwards cells were washed 3 times with FACS buffer and pelleted in between at 600×g for 2 min at 4° C. Anti-human IgG secondary antibody was added for 40 min incubation (4° C.) with conjugated with PE (Invitrogen, H10104, 1:200).

Transcriptomics: To identify differentially expressed genes between HBC and LBC, Affymetrix mouse Chips 2.0St arrays were performed in HBC and LBC and differentially expressed genes were selected based on p-value<0.05, calculated using Student's t-test and false discovery rate analysis. Three technical replicates were used and genes were selected on basis of mean probe intensity >100 in both groups.

GPR116-FNDC4 binding experiments: Pull down: 30 μg of 6×HisFcsFNDC4 or 6×HisFc was bound to 2 mg of anti6×His Dynabeads (Invitrogen), for 25 min at room temperature (RT), under rotation, in 1× binding buffer/wash buffer prepared according to the supplier's protocol. Supernatant was collected and the beads were washed 4× for 2 min each wash, under rotation with 1× binding/wash buffer. In the meantime a 100 cm2 dish, with confluent NIH3T3 cells was lysed in 1× pull-down buffer (as described by the supplier) supplemented with 1% TritonX-100. Cells were scraped and passed through a 25 G syringe 10 times and a 27 G syringe for an additional 10 times, on ice. Finally, cell lysates were sonicated 2× for 10 min at 4° C. in a water bath sonicator and the supernatant was collected after 10 min, centrifugation, at 10000 rpm, 4° C. Protein was quantified by BCA Pierce assay kit (Invitrogen) and 300 μg of supernatant was added on recombinant protein pre-bound beads, for 30 min at RT. The supernatant, containing un-precipitated proteins was collected and beads were washed 4× for 2 min each time, under rotation with 1× binding/wash buffer. Immunoprecipitated proteins were eluted in 100 μL His elution buffer, as described by the supplier, for 15 min, at room temperature, under vigorous shaking. Samples were reduced in β-mercaptoethanol containing sample buffer and boiled (98° C.) for 7 min. 10 μL from each sample was loaded on 7.5% TGX premade mini gel from Biorad and protein was transferred to a PVDF membrane with semi dry transfer, under constant voltage of 10 V for 30 min, using the Trans-Blot Turbo transfer system form BIO-RAD. Membranes were blotted against anti-GPR116, using the anti-GPR116 antibody ab136262, from Abcam.

Lentivirus packaging, infection and stable HEK293 clone cells selection: The lentivirus based expression vector was also constructed using the Gateway system (Invitrogen). Human GPR116 with a C terminal FLAG tag was recombined to plenti6/V5 DEST vector from the pEntry1a GPR116 plasmid. The purified plasmid was then transfected to HEK293FT cells together with packaging plasmids from Invitrogen (ViraPower™ Lentiviral Packaging Mix). 72 h later, the supernatant of the transfected cells containing lentiviral particles was harvested and was used to infect new cells. 72 h after infection with lentivirus particles, cells were exposed to blasticidin (Invitrogen) for stable selection. Medium was changed every 3 days with fresh blasticidin. Two weeks later, the cells were trypsinized and re suspended as single cell suspension into 96 well plates. Thereafter, single cell clones were amplified.

Reporter Luciferase gene assays on 3T3L1 stable reporter cell lines: CRE-, NFAT-RE, SRE- and SRF-luc2P transcription activity luciferase reporters from Promega, cat. no. E8471, E8481, E1340, E1350 were transfected with Lipofectamine 3000 to 3T3L1 fibroblasts (passage 12). 72 h post transfection the media was changed into media with 200 ug/ml hygromycin B (Invitrogen cat. no 10687010) and they were selected for 10-12 days. Clonal cell lines were generated with limited dilution. Media was DMEM+10% FBS+1% P/S+200 ug/ml hygromycin B and was refreshed every two days. For the experiments, cells were differentiated into mature adipocytes with the protocol described before 40. After the stimulation as described at the main text cells were lysed with 1× Reporter lysis buffer-Promega cat. no E3971 and luminescence was read after the addition 1:1 of Steady-Glo® Luciferase Assay System—Promega cat. no E2520 with the Varioscan Lux plate reader, in a white 96-well plate. Experiments were performed at day 8-post differentiation. For the CRE- SRE- and SRF luc2P, prior to stimulation with recombinant FcsFNDC4 and Fc the adipocytes were serum starved in DMEM high glucose for 4-5 h. Stimulation was performed in serum free conditions for additional 3-4 h. For the CRE-luc2P all conditions included 0.5 mM 3-Isobutyl-1-methylxanthine (IBMX) (Sigma: 15879). For the NFAT-RE luc2P stimulation was performed in 10% FBS+ DMEM high glucose for 16 h. As positive controls for assay functionality were used: Forskolin 10 uM (Cay11018-1, Biomol), ionomycin 1 uM (sc-3592, Santa Cruz), Phorbol 12-myristate 13-acetate (PMA) 10-20 ng/ml. For the stimulation of Cre-luc2P reporter adipocytes phosphodiesterase (PDE) inhibitor isobutylmethylxanthine (IBMX) (0.5 mM) was present during the stimulation with rec. proteins.

Construction of sh Gpr116 lentivirus and transduction of pre- and adipocytes: ShRNA lentiviral plasmids (pGFP-C-shlenti) against mouse Gpr116 were purchased from Origene (CAT #: TL517926), and four 29mer shRNA sequences were used for silencing mouse Gpr116 TL517926A

(TL517926A)
(SEQ ID NO.: 13)
5′-tactccattcacaccactgtcatcaacaa-3′
TL517926B (TL517926B)
(SEQ ID NO.: 14)
5′-tcgcagtgttctgccacttcaccaatgca-3′
TL517926C (TL517926C)
(SEQ ID NO.: 12)
5′-cgtcatcttagacaagtctgccttgaact-3′
TL517926D (TL517926D)
(SEQ ID NO.: 15)
5′-tgtggctggtgctatccacgacggtcgct-3′

and a non-effective 29-mer scrambled shRNA cassette in pGFP-C-shLenti Vector, (CAT #: TR30021)

(SEQ ID NO.: 16)
5′-gcactaccagagctaactcagatagtact-3′

was used as a control. For virus production, shRNA lenti vetors were constrasfected overnight, with packaging plasmids psPAX2 (Addgene) and PMD2.G (Addgene) to HEK293FT cells, using Lipofectamine 3000. Twenty-four hours later, media was changed by DMEM 10% FBS containing 1.1% BSA. After 24 h, the supernatant was recovered, filtered with 0.45 mm filters and used to infect differentiated mature primary adipocytes. 1 ml of supernatant was added in 1 well of a 12 well plate for 24 hrs. After that, the media was changed to complete DMEM media. GPR116 was more than 70% compared to the scrambled control and it was achieved as early as 72 hrs post infection.

In vivo insulin signaling studies: On HFD fed mice: Mice were fasted overnight (12-16 h) and subsequently were injected (i.p.) with 5 U/kg Humulin and organs were excised after 8 min and snapped frozen in liquid nitrogen.

Adenoassociated virus (AAV) Knockdown in mice: AAV8-U6-GFP-scrmb-shRNA and AAV8-U6-GFP-shFNDC4 were purchased from Vector Biolabs and injected i.v to 9-10 weeks old mice at 1×10¹² GC per mouse. Mice were given a HFD at 10-11 weeks of age. HFD was 45% fat D12451, Research Diets.

Islet Isolation and Glucose-Stimulated Insulin Secretion Assay (GSIS): Primary islets were isolated from the pancreas of 8-13 week-old C57BL6N male mice via collagenase P (Roche) digestion as described before (Szot et al., (2007), Journal of visualized experiments, 255-255), followed by a centrifugation step using Optiprep density gradient (Sigma). Isolated islets were handpicked twice and incubated in RPMI supplemented with 10% v/v FBS and 1% v/v Penicillin-Streptomycin overnight for recovery. The next day, islets were treated with various concentrations of the commercially available bacterial FNDC4 (Adipogen), the in-house produced mammalian FcsFNDC4 or the corresponding negative controls PBS and Fc-peptide for 24 h. To gain sufficient islet numbers, islets of 2 mice were pooled for each biological replicate. For the GSIS assay, 9 islets of comparable size were transferred per well into a low attachment V-shaped 96-well plate. Islets were incubated in modified Krebs Ringer phosphate HEPES buffer (KRPH; 115 mM NaCl, 4.7 mM KCl, 1.2 mM KH2PO4, 1.2 mM MgSO4*7H2O, 20 mM NaHCO₃20 mM, 16 mM HEPES, 2.56 mM CaCl2*2H2O) supplemented with 0.1% BSA (RIA grade) with various glucose concentrations in the presence of the proteins described above. Exendin-4 served as positive control. After incubation in the presence of 1 mM glucose for 1 h, islets were sequentially incubated with 2.8 mM glucose (low glucose), 16.7 mM glucose (high glucose) for 30 min each. In between the incubation steps, islets were washed twice using KRPH with 2.8 mM glucose. Insulin concentration in the supernatant was assessed using the mouse insulin ELISA kit from ALPCO.

C peptide and insulin measurements: For measurements of C peptide in plasma C peptide quantification kit from CrystalChem was used cat.no: 90050 and for measuring C-peptide in serum the C-peptide quantification kit from ALPCO cat.no:80-CPTMS-E01 was used, according to the manufacturer's description. Insulin was measured with a commercial kit from ALPCO cat. no: 80-INSHU-E01.1.

Cytokines, Adipokines ELISA: ELISA quantification of TNFalpha, Leptin, Adiponectin, Resistin was performed according to the kit's instructions-R&D Systems.

Therapeutic injections of FcsFNDC4 to HFD (60% fat) mice: WT C57BL6N, male mice were fed on a HFD with 60% fat (Research Diets Cat. #D12492) for 16 weeks, starting from 8-9 weeks of age. Mice were given intraperitoneal injections of FcsFNDC4 (0.2 mg/kg) or Vehicle control (PBS) every second day for 4 weeks, while mice still on HFD (60% fat). Glucose clearance and insulin tolerance was assessed with an intraperitoneal glucose tolerance test (IPGTT) and i.p insulin tolerance test (ITT).

2 NBDG (2-(N-(7 Nitrobenz 2 oxa 1,3 diazol 4 yl) 2 Deoxyglucose) tissue uptake quantification: Male C57BL6N mice were on HFD (45% fat) for 20 weeks and the last weeks (16 20 weeks) were injected i.p. every other days with FcsFNDC4 (0.2 mg/kg) or vehicle control. Prior the IPGTT with fluorescent glucose (2 NBDG) mice were fasted for 6 h and were injected i.p with a mix of normal D glucose and 2 NBDG glucose (100× diluted in normal non labelled D glucose). Mice were sacrificed at 35 min after injection, which based on pilot studies was the time point 5 min after all mice had shown a peak in blood glucose (peak was at 30 min). Tissues were collected and snap frozen in liquid N. Tissues were weighed and homogenized in RIPA buffer and fluorescence was measured in a plate reader.

Glucose and insulin tolerance test: Before testing mice were placed in a new cage and food was removed for 6 h. After this period of fasting, the inventors assessed glucose tolerance by intraperitoneal injection (i.p) of 2 g/kg D glucose at time point 0 min and subsequent measurements of blood glucose at 0, 15, 30, 60, 90, 120 and 180 min using ACCU CHEK glucometer strips. To measure glucose induced insulin secretion the inventors collected blood at 0, 15, 30 and 90 min in EDTA coated tubes. Plasma was collected after spinning the blood at 2000×g for 10 min. To assess insulin tolerance the inventors measured blood glucose levels at several times points after i.p injection of insulin. Insulin used was Humulin.

Histology: Liver samples and adipose tissue specimens were fixed in neutrally-buffered 4% formaldehyde solution for 24 hours (Formalin 10% neutral buffered, HT501128, Sigma-Aldrich, Germany) and subsequently routinely embedded in paraffin (Tissue Tec VIP.5, Sakura Europe, Netherlands). Sections of 3 μm nominal thickness were stained with hematoxylin and eosin (HE), using a HistoCore SPECTRA ST automated slide stainer (Leica, Germany) with prefabricated staining reagents (Histocore Spectra H&E Stain System S1, Leica, Germany), according to the manufacturer's instructions. Histopathological examination was performed by a pathologist in a blinded fashion (i.e., without knowledge of the treatment-group affiliations of the examined slides). Immunohistochemical (IHC) detection of CD68 in eWAT and iWAT sections was performed on a Ventana Discovery Ultra-stainer (Roche Diagnostics, Germany), using specific antibodies (polyclonal rabbit anti-CD68 antibody, #125212, Abcam, USA, and secondary antibody: goat anti-rabbit IgG antibody (H+L), biotinylated, BA-1000, Vector, Germany) and prefabricated solutions (DISCOVERY DAB Map Detection Kit, Cat. 760-124, Roche, USA). All IHC analyses included appropriate negative control slides (omission of the first antibody). H&E-stained slides and IHC-sections were digitally scanned with an Axio Scan.Z1 scanner (Zeiss, Germany), using a 20× objective. Automated digital image analysis (Definiens Developer XD 2, Definiens AG, Germany) was used for determination of the mean adipocyte section profile areas, as well as the numbers of CD68-positive macrophage cell section profiles and the percentage of CD68-positive stained area per total adipose tissue section area.

Tissue lipid extraction and TG measurements: Lipid were extracted according to the Folch, J method (Folch, J., Lees, M., and Stanley, G. H. S. (1957). A Simple Method for the Isolation 752 and Purification of Total Lipides from Animal Tissues. J. Biol. Chem. 226, 497-509). Briefly 10 100 mg of frozen wet tissue were weighed, to which 1.5 mL of chloroform:methanol (2:1) mixture (maintained at 80° C.; final volume is about 1212 1.6 mL) were added. Tissues were lysed with the Qiagen TissueLyser (2×30 s, 30 Hz) until no visible large particles remain. The lysed solution was spun down briefly and mix for 20 min on Thermomixer at 1400 rpm, RT and centrifuged for 30 min at 13000 rpm at 20° C. Afterwards 1 mL of supernatant (i.e. liquid phase) was transferred to a new 2 mL tube and add 200 μL of 150 mM (0.9%) NaCl and mix by vigorous shaking and centrifuged for 5 min at 2000 rpm. The resulted lower organic phase was transfer into new tube containing the chloroform:Triton-X (40 μL of chloroform:Triton-X (1:1) solution). This solution was dried with the speedvac overnight (or until no change in tube weight) and the remaining triton-lipid solution was resuspended in 200 μL of dH2O and nixed by 1 h rotation at RT and then stored at −80° C. until use (final volume 225 μL, 1.125 dilution factor). Triglycerides were measured by the Sigma Triglyceride determination kit, Cat. #TR0100.

Tritium 2 Deoxyglucose Uptake Assay: 3T3 L1 adipocytes in 12 well plates were washed twice and incubated with serum and bicarbonate free DMEM containing 20 mM HEPES, pH 7.4, and 0.2% BSA for 2 h. Following 3 h serum starvation, cells were washed twice with Krebs Ringer phosphate buffer (0.6 mM Na2HPO4, 0.4 mM NaH2PO4, 120 mM NaCl, 6 mM KCl, 1 mM CaCl2, 1.2 mM MgSO4, 12.5 mM HEPES, pH 7.4) supplemented with 0.2% BSA. Indicated dose of insulin were added for 20 min, and glucose transport was initiated by the addition of [3H]2 deoxyglucose (PerkinElmer Life Sciences) (0.25 μCi/well, 50 μM unlabeled 2-deoxyglucose) for 5 min. To determine nonspecific glucose uptake, 25 μM cytochalasin B was added prior to the addition of [3H]2-deoxyglucose. Uptake was terminated with three rapid washes in ice-cold PBS, after which the cells were solubilized in 1% Triton X-100 in PBS. Samples were assessed for radioactivity by scintillation counting. Each condition was performed in triplicate. For calculation of uptake non specific uptake was subtracted by the specific uptake and values were normalized for protein concentration.

FNDC4 signaling in 3T3L1 adipocytes and primary mouse SVF derived adipocytes: 3T3L1 differentiation protocol: Differentiation of 3T3L1 to mature adipocytes was done according to the protocol by Zebisch, K., Voigt, V., Wabitsch, M. & Brandsch, M. Protocol for effective differentiation of 3T3-L1 cells to adipocytes. Analytical Biochemistry 425, 88-90 (2012). Experiments were performed of day 8-day 12 of differentiation and passage number between 15-20.

Induction of in vitro insulin resistance and long-term incubation with FcsFNDC4: Insulin resistance was induced on 3T3L1 mature adipocytes by overnight (16 h) exposure to 10 nM insulin, in DMEM high glucose, 10% FBS, 1% P/S (complete media), according to the protocol of Tan et al 14. Different concentrations of FcsFNDC4 or Fc controls were added to the cells for 16 h together with insulin (10 nM), with or without anti-GPR116 (ab111169) or isotype control. Also cells without insulin were included (w/o), as control for the 16 h insulin effect. After 16 h incubation described above media was removed and cells were washed twice with PBS. Cells were incubated in serum free High glucose DMEM for 3 h and then fresh serum free high glucose DMEM containing different concentrations of insulin (0 nM, 0.5 nM, 1 nM) were added to the cells for 5 min. At that time media was removed and cells were washed twice with ice cold PBS and lysed in cell lysis buffer Cat. #9803 with additional protease and phosphatase inhibitors.

For the short-term co stimulation with FcsFNDC4 and insulin: 3T3L1 mature adipocytes were incubated for 3 h in serum free, high glucose DMEM. 30 min before the co stimulation with insulin and FcsFNDC4 anti GPR116 (ab111169) or isotype control was added to the media.

Differentiation of mouse primary SVF to mature adipocytes: Primary SVF cells were isolated by collagenase B digestion of inguinal WAT (after removal of the lymph node) from male mice, 6-8 weeks old. SVF preadipocytes were grown till confluence in DMEM high glucose, 10% FBS+1% P/S, when differentiation was initiated with the addition of Dexamethasone (2 ng/ml), IBMX (122 ng/ml), T3 (6.7 ng/ml), Insulin (0,865 ng/ml) and Rosiglitazone (5 ng/ml) for 2 day. On day 3 media was changed to complete media containing 0,865 ng/ml insulin for 2 days and on day 5 the media was changed in complete media containing 0.4325 ng/ml insulin, in which they were maintained. Experiments were performed between day 6-8 post differentiation.

Western blot: Soluble lysates were collected by centrifugation at 15000×g for 15 min. Samples were boiled for 7 min in Laemmli buffer and loaded to a Tris glycine gel. For receptor blots samples were warmed at 70° C. for 30 min and not boiled. Proteins were transferred to a PVDF membrane with semi dry transfer for 14 min at constant 1.4 mA using the Trans Blot Turbo transfer system form BIO RAD. Membranes were blocked with 5% milk for 30 min and incubated overnight with the primary antibody (1:2000) in 2% milk. Washes were done with 0.1% and 0.05% TBS Tween buffer and secondary antibody was used 1:1000 for 1 h at RT.

Real time quantitative PCR (RT-qPCR): mRNA was extracted with the TRIzol reagent (Invitrogen, ThermoFischer Scientific). 1337 For mouse and cell experiments mRNA 500 ng-1 ug of RNA was amplified with RevertAid First Strand cDNA Synthesis Kit Cat. #K1622. cDNA was PCR amplified with TaqMan Gene Expression Master Mix Cat. #4369016 and PowerUp SYBR Green Master Mix 5×, Cat. #A25777. qPCR primers were designed to span exon-exon sequences to generate a product of 100-200 bp and sequences were derived either from the validated Primerbank (http://pga.mgh.harvard.edu/primerbank) or from published literature. The mRNA levels of each gene were calculated with the ddCt method and normalized for the expression mRNA of the housekeeping gene (as indicated in the figure legends). The Inventors used Applied Biosystems QuantStudio 6 and 7 Flex Real-Time PCR, ThermoFisher.

Quantification and statistical analysis: All values in graphs are presented as mean±SEM. Two-way ANOVA for multiple comparison were used to analyze the data. Significant differences between two groups were evaluated using a two-tailed, unpaired or paired Student's t test as the sample groups displayed a normal distribution and comparable variance (* p<0.05, ** p<1359 0.01, *** p<0.001). For quantification of Western blots the inventors performed band densitometry analysis, using Image Lab (Biorad) of Image J.

### Results

Example 1: Generation of the fusion protein of the invention. The inventors introduced a TEV protease site and a linker between the IgG1 and FNDC4. The purpose of this modification was to be able to remove the IgG1 after purification of the recombinant protein. However, this appeared to be impossible as apparently the TEV site is somehow not accessible to the TEV protease. The inventors thus utilized the protein as it was for injection (i.p.) in vivo (FIG. 1).

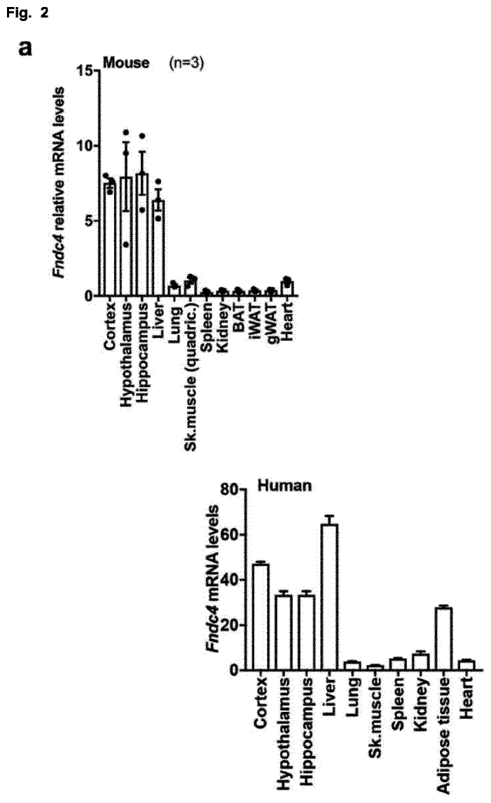

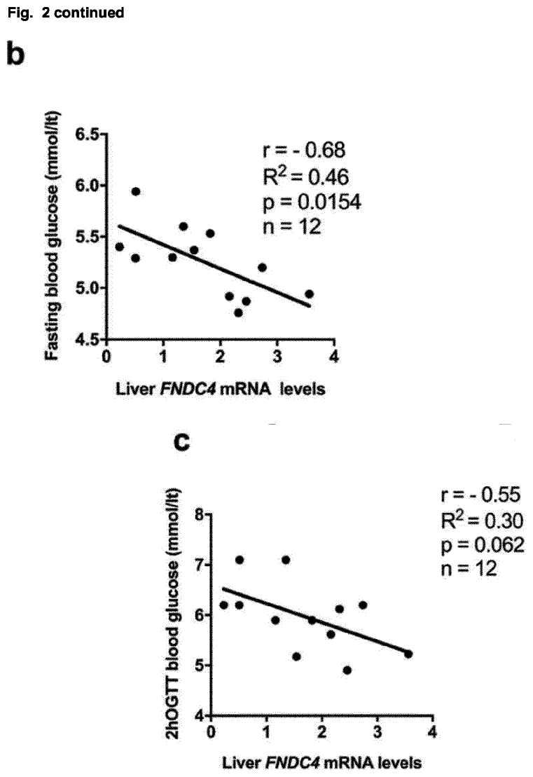

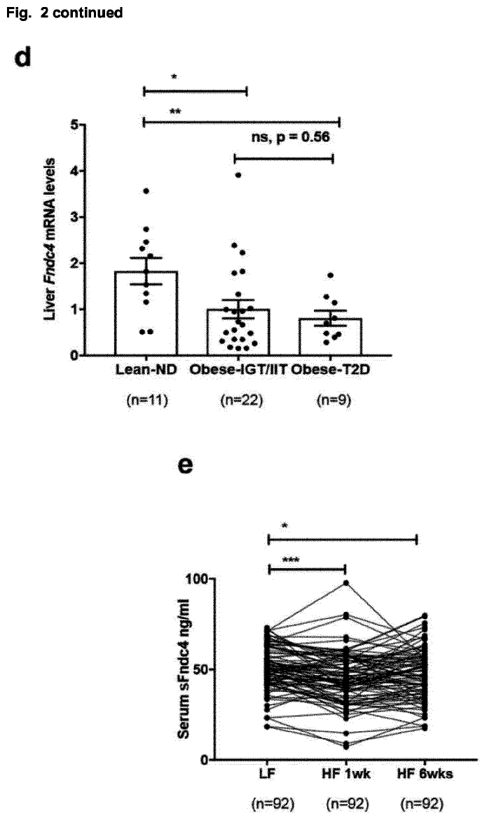

Example 2: Liver and serum FNDC4 levels positively associate with glucose tolerance in humans. Using tissue mRNA profiling, the Inventors found Fndc4 mRNA to be most highly expressed in the liver and brain of mice and humans (FIG. 2a). To investigate the association of FNDC4 with glycemic control in humans, the Inventors measured the mRNA levels of liver FNDC4 from lean and obese humans with or without T2D (see Methods: Cross sectional study-Leipzig). Liver Fndc4 mRNA levels showed an inverse correlation with fasting blood glucose levels (FIG. 2b) and blood glucose levels after a 2 h oral glucose tolerance test (OGTT) (FIG. 2c) in lean healthy individuals. In addition, liver Fndc4 mRNA levels decreased in obese humans with impaired glucose and insulin tolerance (IGT/IIT) and in obese subjects with clinically diagnosed T2D compared to normoglycemic, non-diabetic (ND) lean controls (FIG. 2d).

FNDC4 has been shown to release a soluble peptide (sFNDC4) (Bosma, M., Gerling, M., Pasto, J., Georgiadi, A., Graham, E., Shilkova, O., Iwata, Y., 726 Almer, S., Söderman, J., Toftgård, R., et al. (2016). FNDC4 acts as an anti-inflammatory factor on macrophages and improves colitis in mice. Nat. Commun. 7) and so far there are no reports of sFNDC4 levels in the blood circulation of humans or mice. Therefore, the Inventors quantified sFNDC4 in human serum in a cohort of healthy individuals receiving a high fat diet (HFD), isocaloric to the control diet, for 6 weeks. Blood was collected for analysis after 1 and 6 weeks (see methods: NUGAT study). Under this diet, the participants did not gain weight, but showed an increased HOMA-index, indicating the appearance of insulin resistance in response to the consumed HFD (Schüler, R., Osterhoff, M. A., Frahnow, T., Möhlig, M., Spranger, J., Stefanovski, D., Bergman, R. N., Xu, L., Seltmann, A.-C., Kabisch, S., et al. (2017). Dietary Fat Intake Modulates Effects of a Frequent ACE Gene Variant on Glucose Tolerance with association to Type 2 Diabetes. Sci. Rep. 7, 9234). The Inventors observed a 10% decrease in the serum sFNDC4 levels following 1 week on HFD, which was sustained after 6 weeks on HFD (FIG. 2e). These data supported a positive association between liver Fndc4 mRNA, serum sFNDC4 levels and glucose tolerance as well as insulin sensitivity in humans.

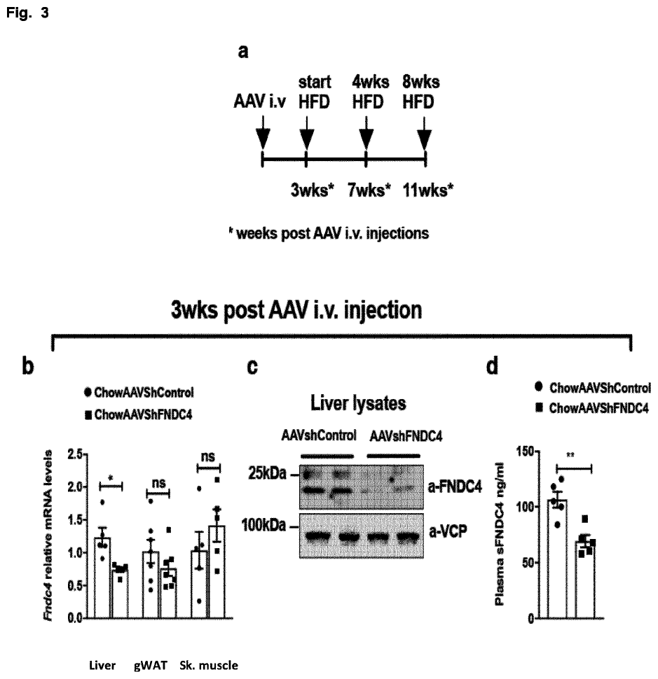

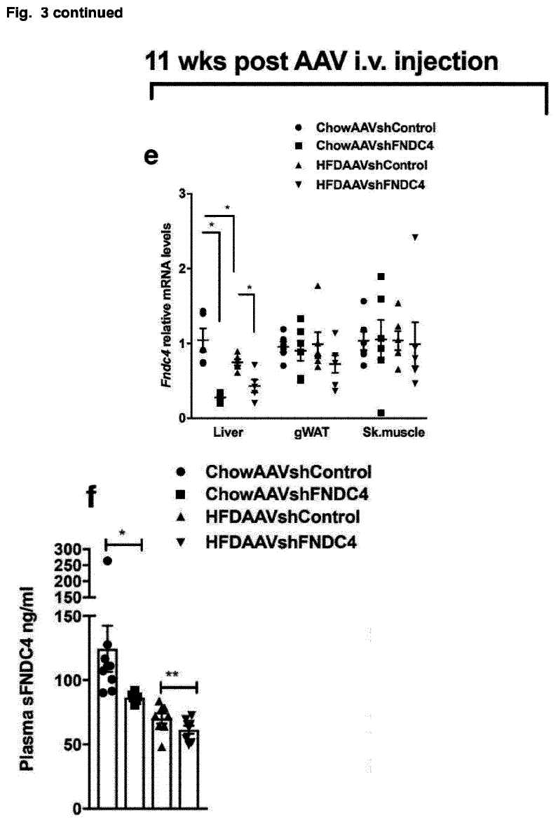

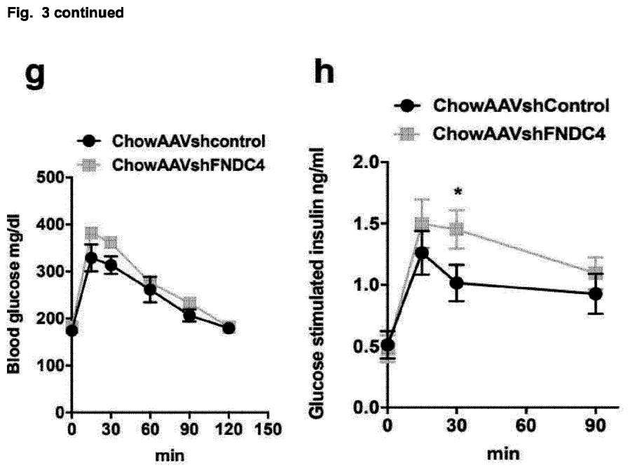

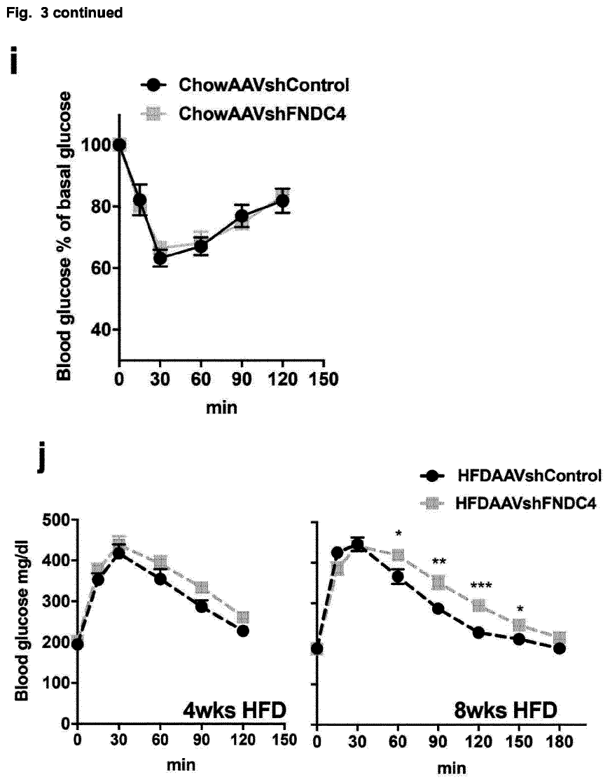

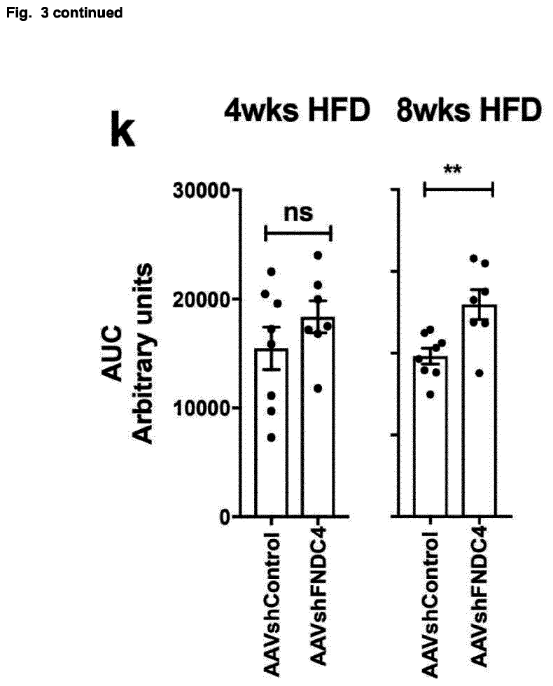

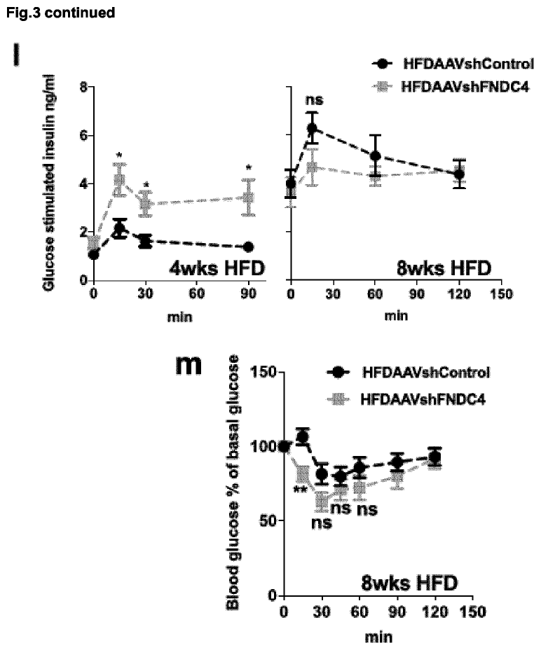

Example 3: Hepatic FNDC4 is required for proper systemic glucose tolerance and specifically targets WAT. To determine the role of hepatic FNDC4 in glucose homeostasis, the Inventors lowered hepatic FNDC4 levels using an AAV8 shFNDC4 specifically targeting the liver. To confirm the knockdown (KD) effect of the AAVs, the Inventors measured liver and circulating FNDC4 3 weeks post AAV injection and then split the AAVshControl and AAVshFNDC4 injected animals into HFD or chow diet groups for a total of 8 weeks (FIG. 3a). 3 weeks after the delivery of AAVshFNDC4, liver Fndc4 mRNA decreased by 40% (FIG. 3b), and both liver FNDC4 protein (FIG. 3c) and FNDC4 plasma levels (FIG. 3d) were significantly reduced. In addition, Fndc4 mRNA was not altered in non-hepatic tissues, such as gonadal WAT (gWAT) and skeletal muscle (gastrocnemius muscle-GC) (FIG. 3b), supporting the notion that the liver represents the main source of circulating FNDC4. Finally, at the end of the study (8 weeks on HFD and total 11 weeks post AAV injections), liver FNDC4 mRNA (FIG. 3e) as well as circulating levels of sFNDC4 still remained significantly reduced in the AAVshFNDC4 group compared to the AAVshControl group under chow and HFD conditions (FIG. 3f). Of note, the Inventors observed substantial differences with regards to the quantified levels of sFNDC4 in mouse plasma derived from trunk blood as opposed to tail blood, with tail plasma measurements of sFNDC4 being up to 10 times lower compared to trunk derived plasma (data not shown).

Under chow diet, AAVshFNDC4 mice showed no difference in glucose clearance during an intraperitoneal glucose tolerance test (IPGTT) (FIG. 3g), however they exhibited compensatory hyperinsulinemia during the IPGTT (FIG. 3h) and showed no significant difference during an insulin tolerance test (ITT) compared to the AAVshControl mice (FIG. 3i). Furthermore, AAVshFNDC4 mice on HFD tended to have higher blood glucose at 4 weeks of HFD and showed impaired glucose clearance at 8 weeks of HFD compared to the AAVshControl animals, during the IPGTT (FIG. 3j, 3k). Importantly, at 4 weeks on HFD AAVshFNDC4 showed severe compensatory hyperinsulinemia during the IPGTT (FIG. 3l). There was no significant difference between the two groups in the ITT at 8 weeks on HFD (FIG. 3m). To examine possible effects ofAAVshFNDC4 liver deletion on insulin secretion, the Inventors also examined the levels of fasting and glucose-stimulated C-peptide and found no difference between AAVshFNDC4 and AAVshControl under chow or HFD conditions (data not shown). Furthermore, the insulin content in the pancreas of AAVshFNDC4 mice was not significantly different from that of the AAVshControl group on chow or HFD (data not shown), nor did recombinant FNDC4 alter insulin secretion in primary pancreatic islets in vitro (data not shown). These findings suggested an intact pancreatic function upon AAVshFNDC4 and argued for an increased peripheral insulin resistance resulting in hyperinsulinemia under those conditions. Finally, no differences in body weight (data not shown), organ weights (data not shown), and food intake (data not shown) between AAVshFNDC4 compared to AAVshControl were observed neither under chow nor under HFD conditions. Furthermore, there were no significant differences in liver or muscle triglyceride (TG) content between AAVshFNDC4 and AAVshControl under chow or HFD diet (data not shown). Serum cholesterol (data not shown), TG (data not shown) and non-esterified fatty acid levels (NEFA) (data not shown) also remained unchanged between AAVshFNDC4 and AAVshControl, under HFD conditions. Overall, these findings demonstrate that decreasing liver and circulating FNDC4 promoted a state of pre-diabetes, manifested by glucose intolerance combined with compensatory hyperinsulinemia and subsequent hyperglycemia.

To identify the tissue target(s) of sFNDC4, the Inventors injected recombinant mammalian, long-lived FcsFNDC4 to HFD mice with glucose intolerance and traced tissue glucose uptake after long-term injections. To determine the injected dose of recombinant FNDC4, the Inventors examined the circulating levels of FNDC4 in mice under chow and HFD feeding. sFNDC4 was present in the circulation throughout the day but tended to peak right several hours before the mice entered the feeding/dark phase (FIG. 5a). Remarkably, HFD feeding reduced the circulating levels of sFNDC4 (FIG. 5a) and decreased liver mRNA levels of Fndc4 (FIG. 5b). the Inventors found that intraperitoneal (i.p) injections of long-lived FcsFNDC4, at a dose of 0.2 mg/kg every second day, recovered the decreased levels of sFNDC4 in the HFD group to physiological levels (chow conditions) (FIG. 5c). Therefore, the Inventors used the dose of 0.2 mg/kg every second day to treat HFD fed mice. Under these conditions, the Inventors observed an improvement in glucose tolerance after 2 weeks (FIG. 5d, 5e), which was maintained for up to 4 weeks upon injections (FIG. 5d, 5e). The Inventors saw no differences in glucose-stimulated insulin secretion (FIG. 5f) and insulin tolerance 24 (ITT) (FIG. 5g) between FcsFNDC4 and vehicle control (VC)-treated mice were observed. Also, body weight (data not shown) and food intake (data not shown) were not altered. Furthermore, the Inventors found no difference in organ weights (data not shown) or in liver and muscle triglycerides (data not shown), which suggested that changes in lipid content in those metabolic tissues could not have accounted for the improved glucose clearance during the IPGTT upon chronic FcsFNDC4 injections.

To examine the tissue contributing to the improved glucose clearance during the IPGTT, the Inventors evaluated the glucose uptake in different tissues using fluorescently labeled glucose (2-NBDG). The Inventors found a significantly higher uptake of fluorescent glucose in the gWAT of HFD mice injected with FcsFNDC4 compared to VC (FIG. 5h). In addition, the Inventors only observed an increase in WAT pAKT levels, of FcsFNDC4 treated HFD mice (4 weeks) compared to VC, after a single intraperitoneal (i.p) injection of insulin, whereas this effect was absent in liver and skeletal muscle (FIG. 5i), supporting an insulin sensitizing effect of FcsFNDC4 specifically in WAT.

Closer histological examination of the gWAT showed no difference in adipocyte size (FIG. 5j) but reduced Cd68-positive cells upon FcsFNDC4 injections (FIG. 5k, 51), suggesting a reduction of macrophages in gWAT of FcsFNDC4 injected mice compared to VC controls. In addition, FscFNDC4 mice had lower total Cd68 mRNA in the gWAT (FIG. 5m). mRNA levels of Resistin, Tnfalpha, Mcp-1, Cc/11, 1110, 243 116, and Cd206 were decreased in gWAT, indicating a reduced inflammatory status in this tissue upon FcFNDC4 delivery (FIG. 5n). Furthermore, FcsFNDC4-treated mice exhibited reduced levels of circulating TNFalpha (FIG. 5o) and Resistin (FIG. 5p), the latter being specifically secreted from WAT in mice (Steppan et al., 2001). The Inventors found no difference in circulating leptin (data not shown) and adiponectin (data not shown) between FcsFNDC4-injected mice compared to VC.

Example 4: Effective dose of FcsFNDC4 towards improving glucose tolerance in HFD mice. The inventors have injected in HFD mice in parallel the FcsFNDC4 published in Bosma et al. 2016 (Bosma, et al. (2016). Nat. Commun. 7) and the FcTEVsFNDC4 of the invention at a dose of 3 mg/kg. Nature Communications 2016, FcsFNDC4 was shown to be bioactive against inflammation at a dose of 3 mg/kg. The Inventors found that the FcsFNDC4 (FcsFNDC4-linker) (Bosma, M., Gerling, M., Pasto, J., Georgiadi, A., Graham, E., Shilkova, O., Iwata, Y., 726 Almer, S., Söderman, J., Toftgård, R., et al. (2016). FNDC4 acts as an anti-inflammatory factor on macrophages and improves colitis in mice. Nat. Commun. 7) worsened glucose tolerance at a dose of 3 mg/kg, every second day injection (i.p) for 1 month injections (8 injections in total), whereas FcTEVsFNDC4 (FcsFNDC4+linker) tended to improve glucose tolerance, but it increased fasting insulin levels compared to the Fc control on HFD C57BL6N mice (FIG. 4). For both proteins no significant effect on the ITT test and body weight after 1 month of injections was found. The Inventors continued then with optimizing the dosage for FcTEVsFNDC4. This novel TEV site containing protein mentioned above was injected in HFD mice in high dose of 3 mg/kg and in low dose 0.2 mg/kg and has been shown to be effective and improved glucose tolerance in HFD in low dose of 0.2 mg/kg (see FIG. 9).

In sum, FcTEVsFNDC4 showed sustained metabolic effects at a dose of 0.2 mg/kg, i.p. injection every other day in HFD mice (see FIG. 5).

Example 5: GPR116 acts as a receptor for soluble FNDC4. To initially identify candidate receptors for sFNDC4, the Inventors set up a fluorescence-readout binding assay in live cells. To this end, the Inventors utilized recombinant sFNDC4, corresponding to the extracellular part of FNDC4 protein (mouse FNDC4 aa: 40-160) fused with human IgG (Fc). The binding of FcsFNDC4 to cells was quantified by detecting the cell-bound ligand (FcsFNDC4) with secondary IgG-PE antibody using fluorescent flow cytometry. For screening, the Inventors chose immortalized mouse pre-adipocytes (imm. WAT) due to their higher survival and robustness during the FACS staining/sorting protocol, the possibility to sort higher numbers and to ensure reproducibility. The Inventors performed saturation binding with increasing concentrations of FcsFNDC4 from 0 nM-500 nM. The Inventors observed increasing levels of fluorescence intensity (Phycoerythrin: PE) following the increasing FcsFNDC4 concentration. By performing a saturation binding curve the Inventors observed saturation of fluorescence readouts around 100 nM of FcsFNDC4. In contrast to FcsFNDC4, the binding of Fc control did not show any saturation of fluorescence (FIG. 6a). The Inventors thus used Fc as a negative control in our assays. Furthermore, by utilizing a competitive FcsFNDC4 binding assay the Inventors observed a concentration-dependent reduction on the binding of FcsFNDC4, supporting the idea of specific binding to a membrane receptor (FIG. 6b).

Next, high and low binding cell populations (HBC, LBC) (FIG. 6c) were sorted, expanded and re-sorted for up to 20 passages to obtain cell populations with stable high and low binding properties to FcsFNDC4 (FIG. 6d). These final high and low binding cell populations were analyzed for differentially expressed genes by Affymetrix transcriptomics analysis. Amongst the top 30 up-regulated genes, the Inventors identified two G-protein coupled receptors and an integrin receptor to be more highly expressed in HBC compared to LBC: relaxin/insulin-like family peptide receptor 1 (Rxfp1, fold change=16), G protein-coupled receptor 116 (Gpr116, fold change=10) and integrin subunit alpha D (ItgaD, fold change=9) (FIG. 6e). RXFP1 ligands are relaxins and insulin-like peptide 3 (INSL3) 10. GPR116 is an orphan adhesion GPCR, and integrin receptors are known to interact with FN3 domain containing proteins. To test the impact of these candidate receptors on FcsFNDC4 binding, the Inventors transiently overexpressed RXFP1 GPR116 or ITGAD in HEK239 cells (FIG. 6g), a cell line that showed much lower baseline binding to FcsFNDC4 than the immortalized pre-adipocytes (data not shown). GPR116 overexpression (OE) increased the binding of FcsFNDC4 compared to control, whereas RXFP1 or ITGAD OE did not have any impact on FcsFNDC4 binding (FIG. 6f). Ligand binding to integrin receptors requires divalent cations such as Ca2+ and Mg2+. Thus, to further test whether an integrin receptor could possibly mediate the binding of FcFNDC4, the Inventors performed FcsFNDC4 binding in the presence of increasing concentrations of EDTA chelator, thereby inhibiting ligand binding to integrins. The Inventors The inventors did not observe any effect of EDTA on FcsFNDC4 binding. Only very high concentrations of EDTA (10 mM) abolished binding (FIG. 6h), overall supporting the hypothesis that GPR116 represents a specific FNDC4 target receptor. Also, the Inventors confirmed the higher expression of GPR116 in HBC versus LBC at the mRNA and protein levels (data not shown).

To further assess the specificity of FcsFNDC4 binding to GPR116 the Inventors performed dose binding of FcsFNDC4 and Fc control to WT, GRP116+/−(HET) and GPR116−/− (KO) SVF derived mouse primary preadipocytes from the inguinal WAT fat depot. The Inventors found that FcsFNDC4 binding decreased upon reduced levels GPR116 mRNA (FIG. 6i) suggesting that expression levels of GPR116 determined the binding of FcsFNDC4. To further assess direct and specific binding of sFNDC4 to GPR116, the Inventors performed a GPR116 pull-down assay using FcsFNDC4 as bait. FcsFNDC4 precipitated GPR116 from total cell lysates of NIH3T3 cells, whereas there was no GPR116 precipitation with the Fc control (FIG. 6j). The Inventors have validated the specificity of the antiGPR116 (ab136262) used to detect GPR116 in FIG. 4j, in NIH3T3 preadipocytes with lenti-ShGPR116 KD and lenti-shControl. At 70% GPR116 KD compared to control cells (data not shown) this antibody show no band close to 250 kDa and a much weaker band a bit higher than 130 kDa (all bands corresponding to the N-terminus of GPR116) (data not shown).

FNDC4 and GPR116 crystal structures do not exist yet. However, based on the protein sequence of GPR116, the extracellular part of this protein contains a predicted GAIN domain. GAIN domains are able to bind FN3 domain containing proteins as seen in the case of the crystal structure of the GAIN domain of GPR56 in complex with a FN3 monobody 23 (data not shown). Therefore, the Inventors employed a GPR116 N-terminal targeting antibody to investigate whether such antibody would abolish FcsFNDC4 binding. The Inventors checked the specificity of this antibody (ab111169) in 3T3L1 mature adipocytes treated with lenti-shGPR116 to induce deletion of endogenous GPR116 (data not shown). Indeed, in HEK293T-GPR1160E cells incubated with anti-GPR116 prior to FcsFNDC4 or Fc binding (100 nM) the Inventors observed an anti-GPR116 dose dependent decrease in the binding of FcsFNDC4 compared to isotype control and Fc control (FIG. 6k). Thus, supporting that FcsFNDC4 binds to the extracellular part of GPR116.

To estimate the binding affinity of FcsFNDC4 to GPR116 the Inventors stably overexpressed GPR116 (human) in HEK293A and HEK293T cells). The Inventors measured GPR116 mRNA levels in HEK293T cells and found no detectable expression of GPR116 in those cells, as opposed to HEK293A cells (HEK293T Ct>32) (FIG. 6l). Using the above described fluorescent flow cytometry binding assay the Inventors created saturation binding curves of FcsFNDC4 and Fc control to HEK cells overexpressing (OE) GPR116 and mock controls (FIG. 6 m,n). In both HEK cell lines only binding of FcsFNDC4 to GPR116 OE cells showed binding saturation (FIG. 6 m,n). In contrast, binding of FcsFNDC4 to mock transfected control was very low and did not saturate in HEK293A cells (FIG. 6m). In HEK293T mock transfected cells, which express no endogenous GPR116, FcsFNDC4 binding was completely absent, similar to Fc control binding (FIG. 6n). Competition binding with excess native sFNDC4 abolished FcsFNDC4 binding, supporting specific binding of FcsFNDC4 in HEK293T GPR116 OE cells. These experiments estimated specific binding of FcsFNDC4 to GPR116 with an equilibrium dissociation constant, Kd=33±10 nM in HEK293A cells (FIG. 6m) and Kd=25±5 nM in HEK293T cells (FIG. 6n).

Example 6: GPR116 is required for the insulin sensitizing effects of FcsFNDC4 in adipocytes. The data thus far suggested that the FNDC4-GPR116 axis may specifically act via liver-WAT communication. To delineate the role of adipose tissue GPR116 in the FcsFNDC4 effects on glucose homeostasis, the inventors generated adipose tissue-specific GPR116 KO mice (GPR116Ad−/−), using adiponectin Cre-mediated gene targeting in GPR116 flox site-carrying mice. HFD-fed GPR116Ad−/− and GPR116Adf/f wild-type mice were treated with FcsFNDC4 or Fc control for 4 weeks by i.p delivery of 0.2 mg/kg every second day. Of note, FcsFNDC4 improved glucose tolerance only in GPR116Adf/f mice, but not in the GPR116Ad−/− littermates, whereas Fc control injections did not have any effect in neither genotype (FIG. 7a, 7b). There were no significant differences of FcsFNDC4 on the glucose-stimulated insulin response (FIG. 7c) and insulin tolerance (FIG. 7d) in neither genotype and in comparison to the Fc-treated. controls. Furthermore, the Inventors did not observe any difference of FcsFNDC4 injections on body (FIG. 7d) and organs weights (FIG. 7f) as compared to the Fc control in both genotypes.

To assess whether the observed anti-inflammatory effects of FcsFNDC4 were also mediated via the adipose tissue GPR116, the Inventors measured circulating resistin (FIG. 7g) and TNFalpha (FIG. 7h). FcsFNDC4 injections decreased these inflammatory markers only in GPR116Adf/f but not in GPR116Ad−/−. Given that GPR116 was not expressed in macrophages and other immune cells (data not shown), (https://www.proteinatlas.org/ENSG00000069122-ADGRF5) our findings supported the conclusion that adipocyte GPR116 mediated both insulin sensitizing and anti-inflammatory effects of FcsFNDC4 in WAT. Importantly, GPR116Ad−/− mice on HFD demonstrated signs of pre-diabetes, manifested by fasting and glucose-stimulated compensatory hyperinsulinemia (FIG. 7c) and tended towards having higher blood glucose levels during an ITT (FIG. 7d) compared to GPR116Adf/f. This phenotype mimicked the effects of decreased hepatic FNDC4 levels (AAVshFNDC4 mice).

To investigate whether sFNDC4 was able to improve insulin resistance in adipocytes and subsequently promote insulin-stimulated glucose uptake, the Inventors utilized a previously described method of in vitro induced insulin resistance in 3T3L1 mature adipocytes (Tan et al., 2015). Exposure of 3T3L1 mature adipocytes to 10 nM insulin for 16 h (overnight-O/N) was sufficient to induce insulin resistance reflected by dampened phosphorylation of downstream effectors of insulin receptor signaling and decreased insulin-dependent glucose uptake upon acute insulin stimulation (Tan, S.-X., Fisher-Wellman, K. H., Fazakerley, D. J., Ng, Y., Pant, H., Li, J., Meoli, C. C., Coster, A. C. F., Stöckli, J., and James, D. E. (2015). Selective Insulin Resistance in Adipocytes. J. Biol. Chem. 290, 11337-11348). In this paradigm, by using low concentrations of FcsFNDC4 concentrations (10 pM-1 nM), co-incubation of FcsFNDC4 with insulin for 16 h (O/N) was able to prevent dampening of insulin signaling due to overnight insulin exposure (FIG. 8a). Similar observations were made for AKT substrate pAS160 which regulates GLUT4 translocation to the cell membrane. This effect peaked at concentrations of 0.25 nM and 0.5 nM, whereas higher concentrations led to dampened signal, possibly suggesting receptor desensitization, a phenomenon typically observed in GPCR activation (Rajagopal and Shenoy, 2018) (FIG. 8a). To explore the role of GPR116 in the FcsFNDC4-dependent effects on insulin-induced pAKT and pAS160, the Inventors employed an antibody targeting the extracellular part of GPR116 (anti-GPR116) (ab111169) (data not shown). This antibody disrupted the binding of FcsFNDC4 to GPR116 OE HEK293T cells compared to the isotype control (FIG. 6k). Upon overnight exposure to insulin, FcsFNDC4 did not improve insulin sensitivity in the presence of anti-GPR116 antibody as it failed to enhance insulin-induced pAKT and pAS160 levels (FIG. 8b). In addition, to exclude secondary effects of the chronic incubation, the Inventors pre-incubated healthy 3T3L1 mature adipocytes with GPR116 blocking antibody for 30 min prior to the addition of fresh media containing only FcsFNDC4 and insulin for 5 min. Also under these acute conditions, FcsFNDC4 enhanced insulin-induced pAKT levels, however failed to do so in adipocytes pre-incubated with anti-GPR116 antibody (data not shown). Importantly, under both chronic and acute conditions, FcsFNDC4 enhanced pAKT and pAS160 levels only in combination with insulin, supporting the notion that FNDC4 acts as a necessary insulin sensitizer in WAT. One of the functional consequences of enhanced insulin signaling in white adipocytes is the promotion of insulin-stimulated glucose uptake via the GLUT4 transporter. To investigate the role of the sFNDC4-GPR116 interaction in insulin-stimulated glucose uptake in 3T3L1 mature adipocytes, the Inventors assessed direct glucose uptake using [3H]2-deoxyglucose (3H-2DG). Under the above-described conditions of insulin resistance in 3T3L1 mature adipocytes, FcsFNDC4 promoted insulin-stimulated glucose uptake, which was absent in the presence of anti-GPR116 antibody (FIG. 8c). Overall, these findings underscored a functional dependence of FcsFNDC4 on GPR116 and they suggested that the interaction of FcsFNDC4 with GPR116 was required for exerting its insulin sensitizing effects in white adipocytes.

Example 7: Interaction of FcsFNDC4 and GPR116 N-terminus induces Gs-cAMP signaling in adipocytes. To investigate if FcsFNDC4 triggered a typical G protein signaling via GPR116, the inventors employed a luciferase reporter assay for G-protein coupling. To that end the inventors generated hygromycin resistant 3T3L1 fibroblast clonal cell lines, each carrying stable expression of transcription reporters: CRE-lu2P, cAMP response element (reporting for Gs signaling), NFAT-RE luc2P, nuclear factor of activated T-cells response element (reporting for Gq signaling), SRE-luc2P, serum response element (reporting Gai signaling) and SRF-luc2P, serum response factor response element (reporting for G12/13 signaling). On day 8 post adipogenic differentiation of each of the above reporter cell lines into mature adipocytes the Inventors performed dose stimulation with FcsFNDC4 or Fc control. The Inventors observed a dose dependent increase in CRE-luc2P activity 3-4 h post induction, whereas Fc control did not induce any increase in luminescence. The Inventors did not observe any change in luminescence in none of the NFAT-RE (16 h post induction), SRE- (3-4 h post induction) or SRF- (3-4 h post induction) reporter carrying adipocytes (FIG. 8d). As a positive control for the assay functionality the Inventors used Forskolin 10 uM for the CRE-luc2P activity, 40% FBS (fetal bovine serum)+20 ng/ml PMA for the SRE-luc2P activity and 40% FBS (fetal bovine serum) for the SRF-luc2P activity. For those reporters, the positive control induction resulted in a significant increase in luminescence compared to control media condition already at 3-4 h post induction, however the induction of the NFAT-RE luc2P activity by ionomycin 1 uM+ PMA 10 ng/ml required 16 h to produce a significant difference in luminescence compared to the control media. Nevertheless, stimulation with FcFNDC4 did not induce NFAT-RE luc2P activity neither after 16 h of incubation (FIG. 8d), nor after 3-4 h of stimulation (data not shown). These findings suggest that sFNDC4 triggers an early Gs-cAMP signaling in white adipocytes, which is consistent with the idea of targeting a GPCR receptor. To assess the dependence of this signaling on the interaction of FcsFNDC4 to GPR116 ectodomain the Inventors performed the same dose induction in the CRE-luc2P 3T3L1 adipocytes, which were incubated with the anti-GPR116 antibody, or isotype control 30 min prior stimulation with FcsFNDC4. The presence of anti-GPR116 antibody inhibited the FcsFNDC4 induced CRE-luc2P activity, whereas FcsFNDC4 indeed induced CRE-luc2P activity in isotype control treated adipocytes (FIG. 8e). The induction of Gs-cAMP signaling by FcsFNDC4-GPR116 was further supported by a rapid and transient induction of cAMP sensitive pCREB and pPKA substrate in response to FcsFNDC4 (FIG. 8 f, g), which was absent in adipocytes preincubated with anti-GPR116 blocking antibody (FIG. 8g). Furthermore, we did not observe any changes in pPKC substrate, in response FcsFNDC4 (data not shown). Therefore, the Inventors concluded that FcsFNDC4-GPR116 activation in white adipocytes leads to Gs coupling and activates cAMP signaling.

### Example 8: Determine the Bioavailability and Half-Life of FcsFNDC4 by Subcutaneous Administration into WT Lean C57BL6J Mice

1 mg/kg of FcsFNDC4 was injected subcutaneously (SC) in WT male mice, C57B16J, 12 weeks old. In a parallel group of mice the inventors injected a reference 1 mg/kg FcsFNDC4 intravenously (IV). The inventors collected plasma at several time points post injection and quantified levels of human IgG1, by ELISA. For the SC group the inventors used n=18 mice, 2 sampling points per animal and 3 mice per time point. Time points of blood collection T=30 min, 1, 2, 4, 8, 24, 48, 72, 96, 144, 168 and 216 hours. For the IV n=21 mice, 2 sampling points per animal and 3 mice per time point. Time points of blood collection T=2 min, 30 min, 1, 2, 4 8, 24, 48, 72, 96, 144, 168, and 216 hours. See Table 2 for the sampling schedule.

TABLE 2
Times scheme of dosing of SC and IV administration of FcsFNDC4 1 mg/kg and blood collection times.

| | | Dos. | 2 | 30 | | | | | | | | | | | |
|---|---|---|---|---|---|---|---|---|---|---|---|---|---|---|---|
| Gr/ROA | Dyr | Time | min | min | 1 h | 2 h | 4 h | 8 h | 24 h | 48 h | 72 h | 96 h | 144 h | 168 h | 216 h |
| 1/SC | 1 | 0:00 | | 0:30 | | | | | 8:00 | | | | | | |
| 1/SC | 2 | 0:01 | | 0:31 | | | | | 8:01 | | | | | | |
| 1/SC | 3 | 0:02 | | 0:32 | | | | | 8:02 | | | | | | |
| 2/IV | 19 | 0:03 | | 0:33 | | | | | 8:03 | | | | | | |
| 2/IV | 20 | 0:04 | | 0:34 | | | | | 8:04 | | | | | | |
| 2/IV | 21 | 0:05 | | 0:35 | | | | | 8:05 | | | | | | |
| 1/SC | 4 | 0:06 | | | | 2:06 | 4:06 | | | | | | | | |
| 1/SC | 5 | 0:07 | | | | 2:07 | 4:07 | | | | | | | | |
| 1/SC | 6 | 0:08 | | | | 2:08 | 4:08 | | | | | | | | |
| 2/IV | 22 | 0:09 | | | | 2:09 | 4:09 | | | | | | | | |
| 2/IV | 23 | 0:10 | | | | 2:10 | 4:10 | | | | | | | | |
| 2/IV | 24 | 0:11 | | | | 2:11 | 4:11 | | | | | | | | |
| 1/SC | 7 | 0:12 | | | | | | | | | | | | | |
| 1/SC | 8 | 0:13 | | | | | | | | 8:12 | 8:12 | | | | |
| 1/SC | 9 | 0:14 | | | | | | | | 8:13 | 8:13 | | | | |
| 2/IV | 25 | 0:15 | | | | | | | | 8:14 | 8:14 | | | | |
| 2/IV | 26 | 0:16 | | | | | | | | 8:15 | 8:15 | | | | |
| 2/IV | 27 | 0:17 | | | | | | | | 8:16 | 8:16 | | | | |
| 1/SC | 10 | 0:18 | | | | | | | | 8:17 | 8:17 | | | | |
| 1/SC | 11 | 0:19 | | | | | | | | | | 8:18 | 8:18 | | |
| 1/SC | 12 | 0:20 | | | | | | | | | | 8:19 | 8:19 | | |
| 2/IV | 28 | 0:21 | 0:23 | | | | | | | | | 8:20 | 8:20 | | |
| 2/IV | 29 | 0:22 | 0:24 | | | | | | | | | | | | 8:21 |
| 2/IV | 30 | 0:23 | 0:25 | | | | | | | | | | | | 8:22 |
| 1/SC | 13 | 0:24 | | | | | | 8:24 | | | | | | | 8:23 |
| 1/SC | 14 | 0:25 | | | | | | 8:25 | | | | | | | 8:24 |
| 1/SC | 15 | 0:26 | | | | | | 8:26 | | | | | | | 8:25 |
| 2/IV | 31 | 0:27 | | | | | | 8:27 | | | | | | | 8:26 |
| 2/IV | 32 | 0:28 | | | | | | 8:28 | | | | | | | 8:27 |
| 2/IV | 33 | 0:29 | | | | | | 8:29 | | | | | | | 8:28 |
| 1/SC | 16 | 0:30 | | | 1:30 | | | | | | | | | | 8:29 |
| 1/SC | 17 | 0:31 | | | 1:31 | | | | | | | | | 8:30 | |
| 1/SC | 18 | 0:32 | | | 1:32 | | | | | | | | | 8:32 | |
| 2/IV | 34 | 0:33 | | | 1:33 | | | | | | | | | 8:32 | |
| 2/IV | 35 | 0:34 | | | 1:34 | | | | | | | | | 8:33 | |
| 2/IV | 36 | 0:35 | | | 1:35 | | | | | | | | | 8:34 | |
| 2/IV | 37 | 0:36 | | | | | | | | | | | | 8:35 | |
| 2/IV | 38 | 0:37 | | | | | | | | | | 8:36 | 8:36 | | |
| 2/IV | 39 | 0:38 | | | | | | | | | | 8:37 | 8:37 | | |
| | | | | | | | | | | | | 8:38 | 8:38 | | |

(Abbreviations: Gr is group, RoA is Route of Administration).

It was found that the total amount of injected FcsFNDC4 had entered the circulation within 48 hrs post injection when administered SC (FIG. 10). In addition, FcsFNDC4 showed 100% bioavailability. SC FcsFNDC4 half-life was determined to be 213 hours or 8.9 days (Table 3).

TABLE 3
Calculated half-life (T_{1/2}) in hours, time when maximum
concentration of injected protein is seen in the blood
(Cmax) in hours and bioavailability of FcsFNDC4 injected
either via the SC or IV route.
FcsFNDC4

| | SC | IV |
|---|---|---|
| T_{1/2} (hours) | 213 | 226 |
| Cₘₐₓ (hours) | 48 | (2 min) |
| Bioavailability | 100% | 100% |

There is no loss of FcsFNDC4 protein when delivered SC (100% bioavailability). In addition, FcsFNDC4 is stable for a long time in the blood circulation and based on the calculated half-life, SC injections of FcsFNDC4 should be performed once every 8.9 days or once weekly.

1. A fusion protein comprising
a) a soluble FNDC4 (sFNDC4) or a functional fragment thereof;
b) a peptide linker; and
c) a Fc-domain.

2. The fusion protein of claim 1, wherein the sFNDC4 comprises an amino acid sequence having at least 70% identity with an amino acid sequence of SEQ ID NO.: 1.

3. The fusion protein of claim 1, wherein the sFNDC4 has the amino acid sequence of SEQ ID NO.: 1.

4. The fusion protein of claim 1, wherein the sFNDC4 has the amino acid sequence of SEQ ID NO.: 2.

5. The fusion protein of claim 1, wherein the fragment is at least about 10 amino acids long.

6. The fusion protein of claim 1, wherein the C-terminal residue of the peptide linker is directly fused to the N-terminus of the sFNDC4.

7. The fusion protein of claim 6, wherein the N-terminal residue of the peptide linker is directly fused to the C-terminal residue of the Fc-domain.

8. The fusion protein of claim 1, wherein the peptide linker comprises between about 5 and about 13 amino acids, optionally about 9 amino acids.

9. The fusion protein of claim 1, wherein the peptide linker comprises a Tobacco Etch Virus (TEV) protease site.

10. The fusion protein of claim 1, wherein the peptide linker comprises the amino acid sequence of SEQ ID NO.: 3.

11. The fusion protein of claim 1, wherein the Fc-domain is selected from the group consisting of an IgG1, IgG2, IgG3 and an IgG4 Fc-domain, optionally wherein the Fc-domain is a human Fc-domain or a mouse Fc-domain.

12. (canceled)

13. (canceled)

14. The fusion protein of claim 1
i) having binding affinity to the G-protein coupled receptor GPR116, optionally wherein the fusion protein specifically binds to the N-terminus of the GPR116 receptor;
ii) having the amino acid sequence of SEQ ID NO.: 4; and/or
iii) having the amino acid sequence of SEQ ID NO.: 5.

15-17. (canceled)

18. A nucleic acid molecule comprising a nucleotide sequence encoding the fusion protein of claim 1.

19-21. (canceled)

22. A method of preventing and/or treating diabetes or inflammation in a subject, the method comprising administering to the subject a therapeutically effective amount of the fusion protein of claim 1, optionally wherein the fusion protein is administered to the subject in a dosage below 3 mg/kg; and/or
wherein said administering is performed by injection or by infusion, optionally wherein the administration is performed:
i) intraperitoneally, optionally wherein said administering comprises at least about 8 administrations, further optionally at least about 8 administrations within one month,
ii) intravenously;
iii) intraarterially;
iv) subcutaneously, optionally wherein said administering comprises administration once a week, further optionally once a week within one month; or
v) intramuscularly.

23-29. (canceled)

30. The method of claim 22, wherein the fusion protein
i) is administered in combination with an additional therapeutic agent;
ii) improves glucose tolerance in the subject; and/or
iii) has binding affinity to the G-protein coupled receptor GPR116, optionally
wherein the fusion protein specifically binds to the N-terminus of the GPR116 receptor; and/or
wherein the GPR116 receptor is located in adipose tissue cells.

31-35. (canceled)

36. The method of claim 22, wherein the subject is a mammal, optionally a human.

37. A composition comprising the fusion protein of claim 1, optionally wherein the composition further comprises at least one diagnostically or pharmaceutically acceptable carrier.

38. (canceled)

39. A kit comprising the fusion protein of claim 1 or a composition comprising said fusion protein.

40. A method of producing the fusion protein of claim 1, comprising producing the fusion protein from a nucleic acid coding for the fusion protein, wherein optionally, producing the fusion protein occurs via genetic engineering, optionally in a bacterial or eukaryotic host organism and the fusion protein is isolated from the host organism or its culture.

41. A method of stratifying a subject with diabetes, comprising
a) determining
the level of sFNDC4 or a fragment thereof in a test sample obtained from said subject, which has been contacted with the fusion protein of any one of claim 1 or a composition comprising said fusion protein, and
b) stratifying said subject as suffering from diabetes, if the level of sFNDC4 is decreased relative to a corresponding level of sFNDC4 in a control sample obtained from a healthy subject.

42. (canceled)


SEQUENCE LISTING (16 sequences)

SEQ ID NO: 1 - protein, 125 aa, Homo Sapiens
  Soluble Fibronectin type III domain-containing protein 4
DRPPSPVNVTVTHLRANSATVSWDVPEGNIVIGYSISQQRQNGPGQRVIREVNTTTRACALWGLAEDSDYTVQVRSIGLRGESPPGPRVHFRTLKGSDRLPSNSSSPGDITVEGLDGERPLQTGE

SEQ ID NO: 2 - protein, 125 aa, Mus musculus; identical to SEQ ID NO: 1
  Soluble Fibronectin type III domain-containing protein 4 of

SEQ ID NO: 3 - protein, 9 aa, Artificial Sequence
  Peptide Linker
  misc_feature (8)..(9): Xaa can be any naturally occurring amino acid
ENLTFQGXX

SEQ ID NO: 4 - protein, 381 aa, Artificial Sequence
  FcsFNDC4 comprising human soluble FNDC4
HHHHHHADLIEGRGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGKAAENLTFQGAARPPSPVNVTVTHLRANSATVSWDVPEGNIVIGYSISQQRQNGPGQRVIREVNTTTRACALWGLAEDSDYTVQVRSIGLRGESPPGPRVHFRTLKGSDRLPSNSSSPGDITVEGLDGERPLQTGE

SEQ ID NO: 5 - protein, 454 aa, Artificial Sequence
  FcsFNDC4 comprising mouse soluble FNDC4
HHHHHHAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGKRPPSPVNVTVTHLRANSATVSWDVPEGNIVIGYSISQQRQNGPGQRVIREVNTTTRACALWGLAEDSDYTVQVRSIGLRGESPPGPRVHFRTLKGSDRLPSNSSSPGDITVEGLDGERPLQTGE

SEQ ID NO: 6 - protein, 242 aa, Homo Sapiens
ADLIEGRGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGKAA

SEQ ID NO: 7 - protein, 324 aa, Mus Musculus
AKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 8 - protein, 7 aa, Artificial Sequence
  TEV protease site
  misc_feature (4)..(4): Xaa is tyrosine or threonine
  misc_feature (7)..(7): Xaa is glycine or serine
ENLXFQX

SEQ ID NO: 9 - protein, 29 aa, Artificial Sequence
  Signal peptide
MPPGPCAWPPRAALRLWLGCVCFALVQAD

SEQ ID NO: 10 - DNA, 31 bp, Artificial sequence
  Forward primer to amplify extracellular part of FNDC4
gagagcggccgctcgacctccctctcctgtg

SEQ ID NO: 11 - DNA, 30 bp, Artificial sequence
  Reverse primer to amplify extracellular part of FNDC4
gagagaattcattcccctgtctgcaatggc

SEQ ID NO: 12 - DNA, 29 bp, Artificial Sequence
  sequences for silencing mouse Gpr116
cgtcatcttagacaagtctgccttgaact

SEQ ID NO: 13 - DNA, 29 bp, Artificial Sequence
  sequences for silencing mouse Gpr116
tactccattcacaccactgtcatcaacaa

SEQ ID NO: 14 - DNA, 29 bp, Artificial Sequence
  sequences for silencing mouse Gpr116
tcgcagtgttctgccacttcaccaatgca

SEQ ID NO: 15 - DNA, 29 bp, Artificial Sequence
  sequences for silencing mouse Gpr116
tgtggctggtgctatccacgacggtcgct

SEQ ID NO: 16 - DNA, 29 bp, Artificial Sequence
  sequences for silencing mouse Gpr116
gcactaccagagctaactcagatagtact